(19) United States
(12) Patent Application Publication
(10) Pub. No.: US 2020/0375987 A1
(43) Pub. Date: Dec. 3, 2020

(54) METALLO-BETA-LACTAMASE INHIBITORS AND METHODS OF USE THEREOF

(71) Applicants: Alexander PASTERNAK, Kenilworth, NJ (US); Shuzhi DONG, Kenilworth, NJ (US); Jack D. SCOTT, Kenilworth, NJ (US); Haiqun TANG, Kenilworth, NJ (US); Zhiqiang ZHAO, Kenilworth, NJ (US); Dexi YANG, Kenilworth, NJ (US); Li XIAO, Kenilworth, NJ (US); Xin GU, Scotch Plains, NJ (US); Merck Sharp & Dohme Corp., Rahway, NJ (US)

(72) Inventors: Alexander Pasternak, Princeton, NJ (US); Shuzhi Dong, Plainsboro, NJ (US); Jack D. Scott, Scotch Plains, NJ (US); Haiqun Tang, Belle Mead, NJ (US); Zhiqiang Zhao, Scotch Plains, NJ (US); Dexi Yang, Livingston, NJ (US); Li Xiao, Cranbury, NJ (US); Xin Gu, Scotch Plains, NJ (US); Jinlong Jiang, Scotch Plains, NJ (US)

(73) Assignee: Merck Sharp & Dohme Corp., Rahway, NJ (US)

(21) Appl. No.: 16/769,670

(22) PCT Filed: Dec. 20, 2018
(86) PCT No.: PCT/US2018/066631

§ 371 Date: Jun. 4, 2020

Related U.S. Application Data
(60) Provisional application No. 62/613,168, filed on Jan. 3, 2018.

(51) Int. Cl. A61K 31/4985; C07D 487/04; C07D 471/04; A61K 31/4439; A61K 31/519; A61K 31/4188; C07D 513/04; C07D 403/10; A61K 31/4178; A61K 31/407; A61K 31/496; A61K 31/165; A61K 31/7048; A61K 31/546; A61P 31/04

(52) U.S. Cl. CPC A61K 31/4985; A61P 31/04; C07D 471/04; A61K 31/4439; A61K 31/519; A61K 31/4188; C07D 513/04; C07D 403/10; A61K 31/4178; A61K 31/407; A61K 31/496; A61K 31/165; A61K 31/7048; A61K 31/546; C07D 487/04

(57) ABSTRACT

The present invention relates to metallo-β-lactamase inhibitor compounds of Formula I: and pharmaceutically acceptable salts thereof, wherein R^{A}, R¹, and Z are as defined herein. The present invention also relates to compositions which comprise a metallo-β-lactamase inhibitor compound of the invention or a pharmaceutically acceptable salt thereof, and a pharmaceutically acceptable carrier, optionally in combination with a beta lactam antibiotic and/or a beta-lactamase inhibitor. The invention further relates to methods for treating a bacterial infection comprising administering to a patient a therapeutically effective amount of a compound of the invention, in combination with a therapeutically effective amount of one or more β-lactam antibiotics and optionally in combination with one or more beta-lactamase inhibitor compounds. The compounds of the invention are useful in the methods described herein for overcoming antibiotic resistance.

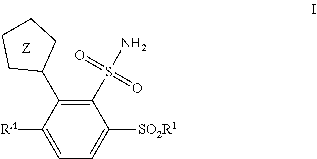

### FIELD OF THE INVENTION

This invention relates to novel metallo-β-lactamase inhibitors and their uses. A preferred use of the metallo-β-lactamase inhibitors is for reducing bacterial beta-lactam antibiotic resistance.

### BACKGROUND OF THE INVENTION

Bacterial antibiotic resistance has become one of the most serious threats to modern health care. Infections caused by resistant bacteria frequently result in longer hospital stays, higher mortality and increased cost of treatment. See, e.g., Cohen, Science 1992, 257:1051-1055. The need for new antibiotics will continue to escalate because bacteria have a remarkable ability to develop resistance to new agents, rendering them quickly ineffective. See, e.g., Neu. Science 1992, 257: 1064-1073. The spread of antibiotic resistance has been referred to as a pandemic. A solution to the growing public health threat will require an interdisciplinary approach. See, e.g., Anderson, Nature America 1999, 5: 147-149. See also Bush et al., Nature Reviews in Microbiology 2011, 9: 894-896; Levy and Marshall, Nature Medicine 2004, 10: S122-S129; Livermore, Clinical Infectious Diseases 2003, 36: S11-S23; and Roberts et al., Clinical Infectious Diseases 2009, 49: 1175-1184.

The present crisis has prompted various efforts to elucidate the mechanisms responsible for bacterial resistance. The widespread use of penicillins and cephalosporins has resulted in the emergence of β-lactamases, a family of bacterial enzymes that catalyze the hydrolysis of the β-lactam ring common to numerous presently used antibiotics. See, Coulton et al., Progress in Medicinal Chemistry 1994, 31: 297-349. This family of bacterial β-lactamases is further divided into four sub-families: A, C, and D families, which comprise β-lactamases that have a serine at the active site that catalyzes the hydrolysis of β-lactam antibiotics, and B family, which comprises β-lactamases that are zinc metalloenzymes. Resistance mediated by β-lactamases is a critical aspect at the core of the development of bacterial antibiotic resistance. See, Dudley, Pharmacotherapy 1995, 15: 9S-14S. Clavulanic acid, which is a metabolite of Streptomyces clavuligerus, and two semi-synthetic inhibitors, sulbactam and tazobactam, are currently available semi-synthetic or natural product β-lactamase inhibitors. Synthetic β-lactamase inhibitors have also been described. See, U.S. Pat. Nos. 5,698,577; 5,510,343; 6,472,406; Hubschwerlen et al., J Med. Chem. 1998, 41: 3961; and Livermore et al., J Med. Chem. 1997, 40: 335-343. Poole (Cell. Mol. Life Sci. 2004, 61: 2200-2223) provides a review of the resistance of bacterial pathogens to β-lactam antibiotics and approaches for overcoming resistance. For a review of inhibitors of metallo β-lactamases, see Fast and Sutton, Biochimica et Biophysica Acta—Proteins and Proteomics 2013, 1834(8): 1648-1659.

U.S. Patent Application Publication No. US 2003/0199541 discloses certain azabicyclic compounds including certain 7-oxo-6-diazabicyclic[3.2.1]octane-2-carboxamides and their use as anti-bacterial agents. U.S. Patent Application Publication No. US 2004/0157826 discloses heterobicyclic compounds including certain diazepine carboxamide and diazepine carboxylate derivatives and their use as anti-bacterials and β-lactamase inhibitors. International Patent Application Publication No. WO 2008/039420 discloses 7-oxo-2,6-diazabicyclo[3.2.0]heptane-6-sulfooxy-2-carboxamides and their use as β-lactamase inhibitors.

Zheng et al. (PLOS One 2013, 8(5), e62955) disclose substituted 2,5-bis-tetrazolylmethyl-thiophenes and their use as β-lactamase inhibitors. Chinese Patent Application Publication No. CN103130686 A discloses N,N′-diaryl-ureas and their use as inhibitors of metallo β-lactamases. Chinese Patent Application Publication No. CN103191091 A discloses substituted arylsulfonamides and their use as inhibitors of metallo β-lactamases.

U.S. Pat. Nos. 4,786,311; 4,746,353; 4,838,925; European Patent Application Publication Nos. EP204513; EP244166; and Chinese Patent Application Publication No. CN1095549A disclose substituted 2-(1H-tetrazol-5-yl)benzenesulfonamides and their use as herbicides.

International Patent Application Publication No. WO 2015/112441 discloses substituted 1H- and 2H-tetrazol-5-yl sulfonamide compounds as metallo β-lactamase inhibitors. WO 2016/210215 discloses 3-tetrazolyl-benzene-1,2-disulfonamide derivatives as metallo-β-lactamase inhibitors. WO 2016/210234 discloses additional compounds as metallo β-lactamase inhibitors.

### SUMMARY OF THE INVENTION

The present invention is directed to substituted 1H- and 2H-tetrazol-5-yl sulfonamide and sulfone compounds and related compounds which are metallo-β-lactamase inhibitors. The compounds, and their pharmaceutically acceptable salts, are useful, for example, in combination with β-lactam antibiotics, and optionally serine β-lactamase inhibitors, for the treatment of bacterial infections, particularly antibiotic-resistant bacterial infections. More particularly, the present invention provides in one embodiment (embodiment no. 1) compounds of Formula I:

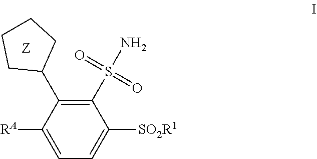

or a pharmaceutically acceptable salt thereof,

wherein:

R^{A} is

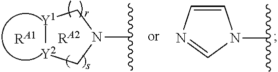

[symbol]

indicates the point of attachment to the rest of the compound,

R^{A1} is a five or six-membered heteroaromatic ring containing 1-3 ring heteroatoms which are independently N, O, or S;

R^{A2} is a five or six-membered heterocyclenyl ring containing 1 or 2 N heteroatoms;

the subscripts r and s are independently 0, 1, 2, or 3, wherein the sum of the subscripts r and s is 2 or 3;

Y¹ and Y² are independently N or C;

wherein R^{A} is optionally substituted with one to three substituents, independently selected from:

(a) —NR^{a}R^{b},

(b) —OR^{a},

(c) —(CH₂)₁₋₃OH,

(d) —C₁-C₆ alkyl, optionally substituted by one to three —F,

(e) —C(O)NR^{a}R^{b}, and

(f) —C(O)OC₁-C₃ alkyl,

Z is tetrazolyl, wherein Z is linked through a carbon to carbon bond to the illustrated phenyl ring;

R is
(1) —NR^{a}—C₁-C₆alkyl, optionally substituted with 1, 2, 3, or 4 substituents independently selected from: —F, —CF₃, —C₁-C₆alkyl, —CH(NH₂)C(O)NH₂, —C(O)NR^{a}R^{b}, —C(O)OH, —(CH₂)₁₋₂NH₂, —NR^{a}(CH₂)₂₋₃NH₂, —NR^{a}R^{b}, —N⁺R^{a}R^{b}CH₃, —NHCH₂CH₂OCH₃, —OR^{a}, and —O(CH₂)₂₋₃NH₂;
(2) —C₁-C₆alkyl, optionally substituted with 1, 2, or 3 substituents independently selected from: —F, —CF₃, —C₁-C₆alkyl, —CH(NH₂)C(O)NH₂, —C(O)NR^{a}R^{b}, —C(O)OH, —(CH₂)₁₋₂NH₂, —NR^{a}(CH₂)₂₋₃NH₂, —NR^{a}R^{b}, —N⁺R^{a}R^{b}CH₃, —NHCH₂CH₂OCH₃, —OR^{a}, and —O(CH₂)₂₋₃NH₂; or
(3) —NR^{a}—(CH₂)ₙ-HetB1;

HetB1 is a 4-6 membered monocyclic, heterocycloalkyl ring with 1 or 2 heteroatom ring atoms independently selected from N, O and S, wherein a N ring atom is optionally in the form of a quaternary amine, and wherein the ring is optionally substituted with 1 or 2 substituents independently selected from —F, —C₁-C₆ alkyl, —C₁-C₆ hydroxyalkyl, —C(O)OR^{a}, —(CH₂)ₖNR^{a}R^{b}, —OR^{a}, and oxo;

R^{a} and R^{b} are independently H or —C₁-C₆ alkyl;
subscript k is 0, 1, 2, 3, or 4; and
each subscript n is independently 0, 1, or 2.

Compounds of Formula I inhibit metallo-β lactamases and can synergize the antibacterial effects of β lactam antibiotics (e.g., imipenem, ceftazidime, ceftolozane, and piperacillin) against microorganisms normally resistant to β lactam antibiotics as a result of the presence of the metallo-β lactamases. Compounds of the present invention are effective against metallo-β lactamases and their combination with a β-lactam antibiotic, such as imipenem, ceftazidime, ceftolozane, or piperacillin, can provide effective treatment of bacterial infections caused by metallo-β lactamase-producing microorganisms. Accordingly, in certain embodiments, the present invention provides compositions comprising a compound of Formula I, IA, or IB with a β-lactam antibiotic, and optionally one or more additional β-lactamase inhibitors, suitable for use against metallo-β lactamase producing bacteria such as Pseudomonas spp. and Klebsiella spp. In some embodiments, the additional one or more β-lactamase inhibitor(s) is a serine (Class A, C and D) β-lactamase inhibitor. The invention also includes compositions comprising a compound of Formula I or a pharmaceutically acceptable salt thereof, and a pharmaceutically acceptable carrier. The invention further includes methods for treating bacterial infections and inhibiting bacterial growth by administration of a compound of Formula I, or a pharmaceutically acceptable salt thereof, to a patient in need thereof, or by administration of a pharmaceutical composition comprising a compound of Formula I or its salt and a pharmaceutically acceptable carrier.

Embodiments, sub-embodiments, aspects and features of the present invention are either further described in or will be apparent from the ensuing description, examples and appended claims.

### DETAILED DESCRIPTION OF THE INVENTION

As noted above, the present invention includes compounds of Formula I, wherein the compounds are metallo-β-lactamase inhibitors suitable for use in combination with β-lactam antibiotics and optionally class A, C, and/or D β-lactamase inhibitors for the treatment of bacterial infections.

The invention is based, in part, on the presence of a sulfur linker ortho to the sulfonamide moiety of the core phenyl ring as a sulfone or sulfonamide. The presence of a sulfur at this position results in improved enzyme potency compared to when the linker is carbon and also provides improved activity on difficult to penetrate Pseudomonas bacterial strains. The improved Pseudomonal activity is likely due to a decrease in efflux from the cells as a result of the sulfone or sulfonamide linker.

In each of the various embodiments of the compounds of the invention described herein, each variable including those of Formula I, and the various embodiments thereof, is selected independently of the other variables unless otherwise indicated. Unless otherwise designated, reference to a compound of the Formula I, as used herein, includes compounds of the Formulas IA and IB.

The present invention encompasses for each of the various embodiments of the compounds of the invention described herein, including those of Formula I, and the various embodiments thereof and the compounds of the examples, all forms of the compounds such as, for example, any solvates, hydrates, stereoisomers, and tautomers of said compounds and of any pharmaceutically acceptable salts thereof, unless otherwise indicated. Additionally, in the examples described herein, the compounds of the invention may be depicted in the salt form. In such cases, it is to be understood that the compounds of the invention include the free acid or free base forms of such salts, and any pharmaceutically acceptable salt of said free acid or free base forms. In addition, in instances where an acidic group such as tetrazole and a basic group such as an amine are present within the same compound, these compounds may be drawn herein for convenience as the free acid and base forms but it should be understood that these can also be alternatively depicted in their zwitterionic forms in which the tetrazole bears a negative charge and the amine bears a positive charge, which are also included as compounds of the invention.

### The Compounds of Formula (I):

In one aspect, the present invention includes compounds of Formula I:

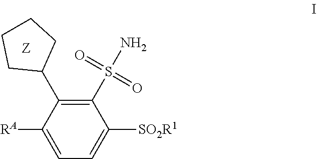

or a pharmaceutically acceptable salt thereof, wherein X₁, X₂, Z, R^{A} and R¹ are as defined herein for the Compounds of Formula (I) in embodiment no. 1 (i.e., as defined in the Summary of the Invention); wherein the compounds may be suitable for use for the treatment of bacterial infections.

In embodiment no. 2, the presentation invention provides a compound of Formula I, or a pharmaceutically acceptable salt thereof, wherein the compound of Formula I has the Formula IA,

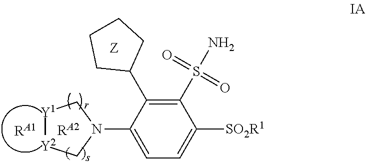

and R¹, R^{A1}, R^{A2}, Y¹, Y², Z and the subscripts r and s are as defined in embodiment no. 1.

In embodiment no. 3, the presentation invention provides a compound of Formula I, or a pharmaceutically acceptable salt thereof, wherein the compound of Formula I has the Formula IB

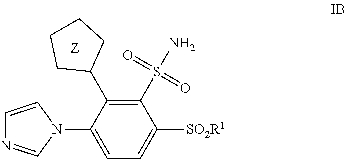

and R¹ and Z are as defined in embodiment no. 1

In embodiment no. 4, the presentation invention provides a compound of Formula IA, or a pharmaceutically acceptable salt thereof, wherein R^{A} is selected from the group consisting of:

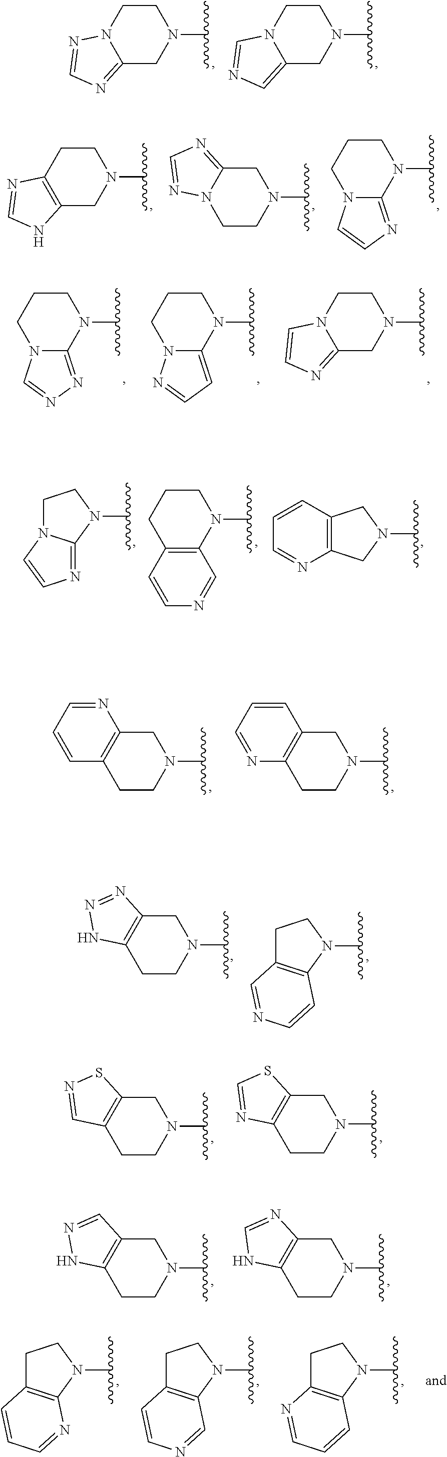

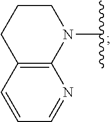 [SMILES: *N1CCCc2cccnc21]

R^{A} is optionally substituted with the substituents set forth in embodiment no. 1.

In embodiment no. 5, the presentation invention provides a compound of Formula IA, or a pharmaceutically acceptable salt thereof, wherein R^{A} is

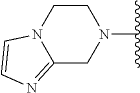 [SMILES: *N1CCn2ccnc2C1]

and is optionally substituted with the substituents set forth in embodiment no. 1.

In embodiment no. 6, the presentation invention provides a compound of Formula I as set forth in any one of embodiment nos. 1-5, or a pharmaceutically acceptable salt thereof, wherein R¹ is:
a) —N(H)—C₂-C₄alkyl, optionally substituted with 1, 2, or 3 substituents independently selected from: —NH₂ and OH;
b) —C₂-C₄alkyl, optionally substituted with 1, 2, or 3 substituents independently selected from —NH₂ and OH; or
c) —N(H)-HetB1, wherein HetB1 is azetidinyl, pyrrolidinyl, or piperidinyl;

In embodiment no. 7, the presentation invention provides a compound of Formula I as set forth in embodiment no. 6, or a pharmaceutically acceptable salt thereof, wherein R¹ is:

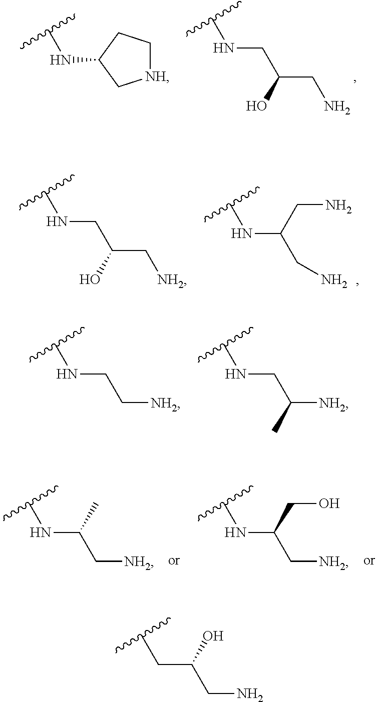

wherein [symbol] indicates the point of attachment to the rest of the compound.

In embodiment no. 8, the present invention provides: (1) a compound having a structure of any of the compounds numbered 1-41 in the Examples herein, (2) the free acid or free base base form (when a basic amine group is present) of any compound numbered 1-41 herein that is depicted as a salt, (3) the zwitterionic form of any of compounds 1-41 which contains a basic amine group, wherein the tetrazole bears a negative charge and the amine group bears a positive charge, or (4) a pharmaceutically acceptable salt of the compounds described in (1), (2), and/or (3).

In embodiment no. 9, the present invention provides: a compound having the structure:

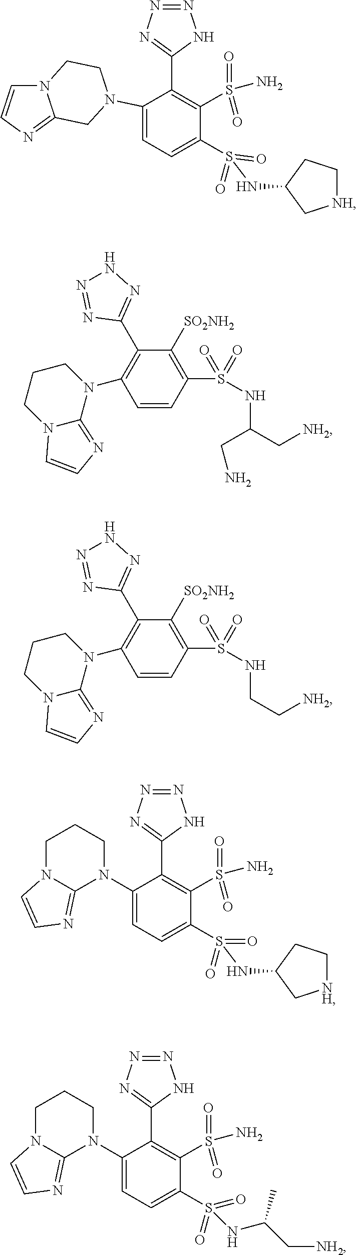 [SMILES: NS(=O)(=O)c1c(S(=O)(=O)N[C@@H]2CCNC2)ccc(N2CCn3ccnc3C2)c1-c1nnn[nH]1 ; NCC(CN)NS(=O)(=O)c1ccc(N2CCCn3ccnc32)c(-c2nn[nH]n2)c1S(N)(=O)=O ; NCCNS(=O)(=O)c1ccc(N2CCCn3ccnc32)c(-c2nn[nH]n2)c1S(N)(=O)=O ; NS(=O)(=O)c1c(S(=O)(=O)N[C@@H]2CCNC2)ccc(N2CCCn3ccnc32)c1-c1nnn[nH]1 ; C[C@H](CN)NS(=O)(=O)c1ccc(N2CCCn3ccnc32)c(-c2nnn[nH]2)c1S(N)(=O)=O]

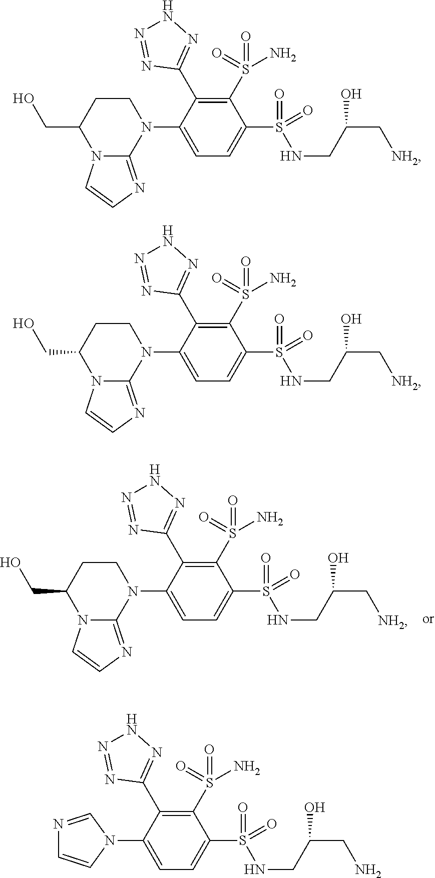 [SMILES: NC[C@@H](O)CNS(=O)(=O)c1ccc(N2CCC(CO)n3ccnc32)c(-c2nn[nH]n2)c1S(N)(=O)=O ; NC[C@@H](O)CNS(=O)(=O)c1ccc(N2CC[C@@H](CO)n3ccnc32)c(-c2nn[nH]n2)c1S(N)(=O)=O ; NC[C@@H](O)CNS(=O)(=O)c1ccc(N2CC[C@H](CO)n3ccnc32)c(-c2nn[nH]n2)c1S(N)(=O)=O ; "or" ; NC[C@@H](O)CNS(=O)(=O)c1ccc(-n2ccnc2)c(-c2nn[nH]n2)c1S(N)(=O)=O]

or a pharmaceutically acceptable salt thereof.

Other embodiments of the present invention include the following:

(a) A pharmaceutical composition comprising an effective amount of a compound of Formula I as defined above, or a pharmaceutically acceptable salt thereof, and a pharmaceutically acceptable carrier.

(b) The pharmaceutical composition of (a), further comprising an effective amount of a β-lactam antibiotic and optionally further comprising an effective amount of a compound which is a class A β-lactamase inhibitor, class C β-lactamase inhibitor, and/or class D β-lactamase inhibitor.

(c) The pharmaceutical composition of (b), wherein the β-lactam antibiotic is selected from the group consisting of: (1) imipenem, (2) ertapenem, (3) meropenem, (4) doripenem, (5) biapenem, (6) panipenem, (7) ticarcillin, (8) ampicillin, (9) amoxicillin, (10) carbenicillin, (11) piperacillin, (12) azlocillin, (13) mezlocillin, (14) ticarcillin, (15) cefoperazone, (16) cefotaxime, (17) ceftriaxone, (18) cefipime, (19) ceftolozane, (20) ceftazidime, and (21) a pharmaceutically acceptable salt of any of (1)-(20), and the class A, C and D β-lactamase inhibitor is selected from the group consisting of relebactam or a pharmaceutically acceptable salt thereof, avibactam or a pharmaceutically acceptable salt thereof, vaborbactam or a pharmaceutically acceptable salt thereof, tazobactam or a pharmaceutically acceptable salt thereof, sulbactam or a pharmaceutically acceptable salt thereof, clavulanic acid or a pharmaceutically acceptable salt thereof, or CB-618 or a pharmaceutically acceptable salt thereof.

(d) The pharmaceutical composition of (b), wherein the β-lactam antibiotic is imipenem or a pharmaceutically acceptable salt thereof.

(e) The pharmaceutical composition of (b), wherein the β-lactam antibiotic is ceftazidime or a pharmaceutically acceptable salt thereof.

(f) The pharmaceutical composition of (b), wherein the β-lactam antibiotic is ceftolozane or a pharmaceutically acceptable salt thereof.

(g) The pharmaceutical composition of (b), wherein the β-lactam antibiotic is piperacillin or a pharmaceutically acceptable salt thereof.

(h) The pharmaceutical composition of (a), further comprising a compound which is a class A β-lactamase inhibitor, class C β-lactamase inhibitor, and/or class D β-lactamase inhibitor.

(i) The pharmaceutical composition of any of (b)-(h), wherein the β-lactamase inhibitor compound is relebactam or a pharmaceutically acceptable salt thereof.

(j) The pharmaceutical composition of any of (b)-(h), wherein the β-lactamase inhibitor compound is tazobactam or a pharmaceutically acceptable salt thereof.

(k) The pharmaceutical composition of (a), further comprising effective amounts of a β-lactam antibiotic, a renal dehydropeptidase (DHP) inhibitor, and optionally, a class A, C and D β-lactamase inhibitor.

(l) The pharmaceutical composition of (k), wherein the β-lactam antibiotic is imipenem or a pharmaceutically acceptable salt thereof, the DHP inhibitor is cilastatin or a pharmaceutically acceptable salt thereof, and the class A, C and D β-lactamase inhibitor is relebactam or a pharmaceutically acceptable salt thereof.

(m) A combination of effective amounts of a compound of Formula I as defined above, or a pharmaceutically acceptable salt thereof, a β-lactam antibiotic, and optionally, a class A, C and/or D β-lactamase inhibitor.

(n) The combination of (j), wherein the β-lactam antibiotic is selected from the group consisting of: (1) imipenem, (2) ertapenem, (3) meropenem, (4) doripenem, (5) biapenem, (6) panipenem, (7) ticarcillin, (8) ampicillin, (9) amoxicillin, (10) carbenicillin, (11) piperacillin, (12) azlocillin, (13) mezlocillin, (14) ticarcillin, (15) cefoperazone, (16) cefotaxime, (17) ceftriaxone, (18) cefipime, (19) ceftolozane, (20) ceftazidime, and (21) a pharmaceutically acceptable salt of any of (1)-(20).

(o) The combination of (n), wherein the β-lactam antibiotic is imipenem or a pharmaceutically acceptable salt thereof, optionally in combination with cilistatin or a pharmaceutically acceptable salt thereof, and the class A, C, D β-lactamase inhibitor is relebactam or a pharmaceutically acceptable salt thereof.

(p) The combination of (n), wherein the β-lactam antibiotic is ceftazidime or a pharmaceutically acceptable salt thereof and the class A, C, D β-lactamase inhibitor is avibactam or a pharmaceutically acceptable salt thereof.

(q) The combination of (n), wherein the β-lactam antibiotic is ceftolozane or a pharmaceutically acceptable salt thereof and the class A, C, D β-lactamase inhibitor is avibactam or a pharmaceutically acceptable salt thereof or relebactam or a pharmaceutically acceptable salt thereof.

(r) The combination of (n), wherein the β-lactam antibiotic is piperacillin or a pharmaceutically acceptable salt thereof.

(s) A combination of effective amounts of a compound of Formula I, as defined above, or a pharmaceutically acceptable salt thereof, and a class A, C and/or D β-lactamase inhibitor.

(t) A combination of effective amounts of a compound of Formula I, as defined above, or a pharmaceutically acceptable salt thereof, a β-lactam antibiotic, a DHP inhibitor, and optionally a class A, C and/or D β-lactamase inhibitor.

(u) The combination of (t), wherein the β-lactam antibiotic is imipenem, the DHP inhibitor is cilastatin or a pharmaceutically acceptable salt thereof, and the class A, C and D β-lactamase inhibitor is relebactam or a pharmaceutically acceptable salt thereof.

(v) A method for treating a bacterial infection which comprises administering to a subject in need of such treatment a therapeutically effective amount of a compound of Formula I as defined above, or a pharmaceutically acceptable salt thereof, in combination with an effective amount of a β-lactam antibiotic and optionally in combination with a class A, C and D β-lactamase inhibitor.

(w) A method for treating a bacterial infection which comprises administering to a subject in need of such treatment a therapeutically effective amount of a compound of Formula I as defined above, or a pharmaceutically acceptable salt thereof, in combination with effective amounts of a β-lactam antibiotic and a DHP inhibitor, and optionally in combination with a class A, C and D β-lactamase inhibitor.

(x) A method for treating a bacterial infection which comprises administering to a subject in need of such treatment a therapeutically effective amount of the composition of (a), (b), (c), (d), (e), (f), (g), (h), (i), (j), (k), or (l).

(y) A method for treating a bacterial infection which comprises administering to a subject in need of such treatment a therapeutically effective amount of the combination of (m), (n), (o), (p), (q), (r), (s), (t), or (u).

(z) A method of treating a bacterial infection as set forth in (v), (w), (x), (y) or (z) wherein the bacterial infection is due to Pseudomonas spp., Klebsiella spp., Enterobacter spp., Escherichi spp.a, Morganella spp., Citrobacter spp., Serratia, spp. or Acintetobacter spp.

The present invention also includes a compound of Formula I or a pharmaceutically acceptable salt thereof, (i) for use in, (ii) for use as a medicament for, or (iii) for use in the preparation (or manufacture) of a medicament for, inhibiting beta-lactamase activity or treating bacterial infection. In these uses, the compounds of the present invention can optionally be employed in combination with one or more β-lactam antibiotics, and may further be employed in combination with a class A, C, and/or D serine β-lactamase inhibitor and/or one or more DHP inhibitors.

Additional embodiments of the invention include the pharmaceutical compositions, combinations and methods set forth in (a)-(z) above and the uses set forth in the preceding paragraph, wherein the compound of the present invention employed therein is a compound of one of the embodiments, sub-embodiments, classes or sub-classes described above. The compound may optionally be used in the form of a pharmaceutically acceptable salt in these embodiments. In addition, the compound may optionally be used in the form of a prodrug that releases the active parent compound after dosing by intravenous or oral administration.

In the embodiments of the compounds and salts provided above, it is to be understood that each embodiment may be combined with one or more other embodiments, to the extent that such a combination provides a stable compound or salt and is consistent with the description of the embodiments. It is further to be understood that the embodiments of compositions and methods provided as (a) through (z) above are understood to include all embodiments of the compounds and/or salts, including such embodiments as result from combinations of embodiments.

Additional embodiments of the present invention include each of the pharmaceutical compositions, combinations, methods and uses set forth in the preceding paragraphs, wherein the compound of the present invention or its salt employed therein is substantially pure. With respect to a pharmaceutical composition comprising a compound of Formula I or its salt and a pharmaceutically acceptable carrier and optionally one or more excipients, it is understood that the term “substantially pure” is in reference to a compound of Formula I, or its salt per se, i.e., the purity of the active ingredient in the composition.

### Definitions and Abbreviations

The term “β-lactamase inhibitor” refers to a compound which is capable of inhibiting enzyme activity from β-lactamases. As used herein, inhibiting β-lactamase activity means inhibiting the activity of a class A, B, C, and/or D β-lactamase. For antimicrobial applications inhibition at a 50% inhibitory concentration is preferably achieved at or below about 100 micrograms/mL, or at or below about 50 micrograms/mL, or at or below about 25 micrograms/mL. The terms “class A”, “class B”, “class C”, and “class D” β-lactamases are understood by those skilled in the art and are described in S. G. Waley, β-lactamase: mechanisms of action, in The Chemistry of β-Lactams, M. I. Page, Ed.; Chapman and Hall, London, (1992) 198-228.

The term “metallo-β-lactamase inhibitor” refers to a compound which is capable of inhibiting metallo-β-lactamase activity. As used herein, inhibiting metallo-β-lactamase activity means inhibiting the activity of a class B metallo-β-lactamase. For antimicrobial applications inhibition at a 50% inhibitory concentration is preferably achieved at or below about 100 μg/mL, or at or below about 50 μg/mL, or at or below about 25 μg/mL.

The term “metallo-β-lactamase” denotes a metalloprotein capable of inactivating a β-lactam antibiotic. The β-lactamase can be an enzyme which catalyzes the hydrolysis of the β-lactam ring of a β-lactam antibiotic. Of particular interest herein are microbial metallo-β-lactamases. The metallo-β-lactamase can be, for example, a zinc metallo-β-lactamase. β-Lactamases of interest include those disclosed in, e.g., S. G. Waley, β-lactamase: mechanisms of action, in The Chemistry of β-Lactams, M.I. Page, Ed.; Chapman and Hall, London, (1992) 198-228. β-Lactamases of particular interest herein include a metallo-β-lactamases of Escherichia coli (such as New Delhi Metallo-β-lactamase, NDM), Serratia marcescens (such as IMP), Klebsiella spp. and Pseudomonas spp (such as Verona integron-encoded metallo-β-lactamase, VIM)). Additional metallo-β-lactamases of interest herein include SPM-, GIM-, SIM-, KHM-, AIM-, DIM-, SMB-, TMB-, and FIM-type enzymes.

The term “antibiotic” refers to a compound or composition which decreases the viability of a microorganism, or which inhibits the growth or proliferation of a microorganism. The phrase “inhibits the growth or proliferation” means increasing the generation time (i.e., the time required for the bacterial cell to divide or for the population to double) by at least about 2-fold. Preferred antibiotics are those which can increase the generation time by at least about 10-fold or more (e.g., at least about 100-fold or even indefinitely, as in total cell death). As used in this disclosure, an antibiotic is further intended to include an antimicrobial, bacteriostatic, or bactericidal agent. Examples of antibiotics suitable for use with respect to the present invention include penicillins, cephalosporins and carbapenems.

The term “β-lactam antibiotic” refers to a compound with antibiotic properties that contains a β-lactam functionality. Non-limiting examples of β-lactam antibiotics useful with respect to the invention include penicillins, cephalosporins, penems, carbapenems, and monobactams and their pharmaceutically acceptable salts.

The term “about”, when modifying the quantity (e.g., kg, L, or equivalents) of a substance or composition, or the value of a physical property, or the value of a parameter characterizing a process step (e.g., the temperature at which a process step is conducted), or the like refers to variation in the numerical quantity that can occur, for example, through typical measuring, handling and sampling procedures involved in the preparation, characterization and/or use of the substance or composition; through inadvertent error in these procedures; through differences in the manufacture, source, or purity of the ingredients employed to make or use the compositions or carry out the procedures; and the like. In certain embodiments, “about” can mean a variation of 0.1, 0.2, 0.3, 0.4, 0.5, 1.0, 2.0, 3.0, 4.0, or 5.0 of the appropriate unit. In certain embodiments, “about” can mean a variation of 1%, 2%, 3%, 4%, 5%, 10%, or 20%.

Another embodiment of the present invention is a compound of Formula I, or a pharmaceutically acceptable salt thereof, as originally defined or as defined in any of the foregoing embodiments, sub-embodiments, aspects, classes or sub-classes, wherein the compound or its salt is in a substantially pure form. As used herein “substantially pure” means suitably at least about 60 wt. %, typically at least about 70 wt. %, preferably at least about 80 wt. %, more preferably at least about 90 wt. % (e.g., from about 90 wt. % to about 99 wt. %), even more preferably at least about 95 wt. % (e.g., from about 95 wt. % to about 99 wt. %, or from about 98 wt. % to 100 wt. %), and most preferably at least about 99 wt. % (e.g., 100 wt. %) of a product containing a compound of Formula I, or its salt (e.g., the product isolated from a reaction mixture affording the compound or salt) consists of the compound or salt. The level of purity of the compounds and salts can be determined using a standard method of analysis such as thin layer chromatography, gel electrophoresis, high performance liquid chromatography, and/or mass spectrometry. If more than one method of analysis is employed and the methods provide experimentally significant differences in the level of purity determined, then the method providing the highest level of purity governs. A compound or salt of 100% purity is one which is free of detectable impurities as determined by a standard method of analysis.

With respect to a compound of the invention which has one or more asymmetric centers and can occur as mixtures of stereoisomers, a substantially pure compound can be either a substantially pure mixture of the stereoisomers or a substantially pure individual diastereomer or enantiomer unless expressly depicted otherwise. The present invention encompasses all stereoisomeric forms of the compounds of Formula I. Unless a specific stereochemistry is indicated, the present invention is meant to comprehend all such isomeric forms of these compounds. Centers of asymmetry that are present in the compounds of Formula I can all independently of one another have (R) configuration or (S) configuration. When bonds to the chiral carbon are depicted as straight lines in the structural Formulas of the invention, it is understood that both the (R) and (S) configurations of the chiral carbon, and hence both enantiomers and mixtures thereof, are embraced within the Formula. Similarly, when a compound name is recited without a chiral designation for a chiral carbon, it is understood that both the (R) and (S) configurations of the chiral carbon, and hence individual enantiomers, diastereomers and mixtures thereof, are embraced by the name. The production of specific stereoisomers or mixtures thereof may be identified in the Examples where such stereoisomers or mixtures were obtained, but this in no way limits the inclusion of all stereoisomers and mixtures thereof from being within the scope of this invention.

The invention includes all possible enantiomers and diastereomers and mixtures of two or more stereoisomers, for example mixtures of enantiomers and/or diastereomers, in all ratios. Thus, enantiomers are a subject of the invention in enantiomerically pure form, both as levorotatory and as dextrorotatory antipodes, in the form of racemates and in the form of mixtures of the two enantiomers in all ratios. In the case of a cis/trans isomerism the invention includes both the cis form and the trans form as well as mixtures of these forms in all ratios. The preparation of individual stereoisomers can be carried out, if desired, by separation of a mixture by customary methods, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of a compound of Formula I or it can be done on a final racemic product. Absolute stereochemistry may be determined by X-ray crystallography of crystalline products or crystalline intermediates which are derivatized, if necessary, with a reagent containing a stereogenic center of known configuration. Unless a particular isomer, salt, solvate (including hydrates) or solvated salt of such racemate, enantiomer, or diastereomer is indicated, the present invention includes all such isomers, as well as salts, solvates (including hydrates) and solvated salts of such racemates, enantiomers, diastereomers and mixtures thereof.

“Alkyl” means saturated carbon chains which may be linear or branched or combinations thereof, unless the carbon chain is defined otherwise. Other groups having the prefix “alk”, such as alkoxy and alkanoyl, also may be linear or branched, or combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like.

“Aminoalkyl” means saturated carbon chains which may be linear or branched or combinations thereof which are substituted with one amino group which may be terminal (—NH₂) or internal (—NH—).

“Hydroxyalkyl” means saturated carbon chains which may be linear or branched or combinations thereof which are substituted with one hydroxyl (—OH) group.

“Diaminoalkyl” means saturated carbon chains which may be linear or branched or combinations thereof which are substituted with two amino (—NH₂) groups.

“Dihydroxyalkyl” means saturated carbon chains which may be linear or branched or combinations thereof which are substituted with two hydroxyl (—OH) groups.

“Hydroxyaminoalkyl” means saturated carbon chains which may be linear or branched or combinations thereof which are substituted with one hydroxyl (—OH) group and one amino (—NH₂) group.

“Alkenyl” means carbon chains which contain at least one carbon-carbon double bond, and which may be linear or branched, or combinations thereof, unless otherwise defined. Examples of alkenyl include vinyl, allyl, isopropenyl, pentenyl, hexenyl, heptenyl, 1-propenyl, 2-butenyl, 2-methyl-2-butenyl, and the like.

“Aromatic ring system” means monocyclic, bicyclic or tricyclic aromatic ring or ring system containing 5-14 ring atoms, wherein at least one of the rings is aromatic. The term may be used to describe a carbocyclic ring fused to an aryl group. For example, a 5-7-membered cycloalkyl can be fused through two adjacent ring atoms to a 5-6-membered heteroaryl containing 1, 2, or 3 heteroatom ring atoms selected from N, O, and S. In other example, a heteromonocyclic ring is fused through two ring atoms to a phenyl or 5-6-membered heteroaryl containing 1, 2, or 3 heteroatoms selected from N, O, and S. In the case of a heteromonocyclic ring containing one or more N atoms, the N can be in the form of quarternary amine. In certain embodiments, a N ring atom can be in the form of an N-oxide.

“Aryl” means a monocyclic, bicyclic or tricyclic carbocyclic aromatic ring or ring system containing 5-14 carbon atoms, wherein at least one of the rings is aromatic. Examples of aryl include phenyl and naphthyl. In one embodiment of the present invention, aryl is phenyl.

“Cycloalkyl” means a saturated monocyclic, bicyclic or bridged carbocyclic ring, having a specified number of carbon atoms. Examples of cycloalkyl include cyclopropyl, cyclobutyl, cyclopentyl, cyclohexyl, cycloheptyl, indanyl, 1,2,3,4-tetrahydronaphthyl and the like. In one embodiment of the present invention, cycloalkyl is selected from: cyclopropane, cyclobutane, cyclopentane and cyclohexane.

“Cycloalkenyl” means a nonaromatic monocyclic or bicyclic carbocylic ring containing at least one double bond. Examples of cycloalkenyl include cyclopropenyl, cyclobutenyl, cyclopentenyl, cyclohexenyl, cycloheptenyl, cyclooxtenyl and the like.

“Cycloheteroalkyl” or “heterocycloalkyl” means a saturated, non-aromatic monocyclic, bicyclic (including spirocyclic) or bridged carbocyclic ring or ring system comprising 3 to about 11 ring atoms, containing at least one ring heteroatom selected from N, S and O and the remainder of the ring atoms are carbon atoms. The nitrogen or sulfur atom of the heterocycloalkyl can be optionally oxidized to the corresponding N-oxide, S-oxide or S-dioxide. A heterocycloalkyl group can be joined via a ring carbon, or ring nitrogen atom, unless specified otherwise. The cycloheteroalkyl ring may be substituted on the ring carbons and/or the ring nitrogen(s). In one embodiment, a heterocycloalkyl group is monocyclic and has from about 3 to about 7 ring atoms (a “3 to 7-membered monocyclic heterocycloalkyl” group). In another embodiment, a heterocycloalkyl group is monocyclic has from about 4 to about 7 ring atoms (a “4 to 7-membered monocyclic heterocycloalkyl” group). In other embodiments, the heterocycloalkyl group is bicyclic and has 7-10 ring atoms, 8-10 ring atoms, or 9 or 10 ring atoms (a “9 or 10-membered bicyclic heterocycloalkyl” group). In still another embodiment, a heterocycloalkyl group is monocyclic and has 5 or 6 ring atoms. In one embodiment, a heterocycloalkyl group is monocyclic. There are no adjacent oxygen and/or sulfur atoms present in the ring system. Examples of cycloheteroalkyl include tetrahydrofuran, piperazine, piperidine, morpholine, and pyrrolidine. Where the ring or ring system contains one or more N atoms, the N can be in the form of quarternary amine.

A “heterocycloalkenyl ring” refers to a 5- or 6-membered, non-aromatic monocyclic ring containing at least one N atom as a ring member and at least one double bond. The heterocyclenyl ring is optionally substituted with one to three substituents. Examples of heterocyclenyl rings include pyrroline, imidazoline, tetrahydropyridine, dihydropyrazine, and tetrahydropyrimidine.

“Heteroaryl” or “heteroaromatic ring” means monocyclic aromatic ring containing containing at least one ring heteroatom selected from N, S (including SO and SO₂) and O. In the case of a heteroaryl ring system. Examples of heteroaryl rings include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, and pyrazinyl.

“Halogen” includes fluorine, chlorine, bromine and iodine.

“Oxo” means an oxygen atom connected to another atom by a double bond and is represented by “═O” herein.

Where any amine is present in the compound, the N atom may be optionally in the form of a quaternary amine having one or more appropriate additional substitutions, as further described herein.

When any ring atom is specified as being optionally substituted with, or in a specified form, for example, S substituted with oxo groups, or N in the form of a N-oxide, this does not preclude the substitution of any ring atom with the other listed optional substituents when not substituted with oxo groups or in the form of a N-oxide.

When any variable (e.g., n, R^{a}, R^{b}, etc.) occurs more than one time in any constituent or in Formula I, its definition on each occurrence is independent of its definition at every other occurrence. Also, combinations of substituents and/or variables are permissible only if such combinations result in stable compounds.

A wavy line [symbol] , as used herein, indicates a point of attachment to the rest of the compound. Lines drawn into a ring system, for example:

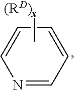

indicate that the bond may be attached to any of the substitutable ring atoms.

Under standard nomenclature used throughout this disclosure, the terminal portion of the designated side chain is described last, preceded by the adjacent functionality toward the point of attachment.

In choosing compounds of the present invention, one of ordinary skill in the art will recognize that the various substituents, i.e. R¹, R^{A}, etc., are to be chosen in conformity with well-known principles of chemical structure connectivity and stability.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

In the compounds of Formula I, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of Formula I. For example, different isotopic forms of hydrogen (H) include protium (¹H) and deuterium (²H or D). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within Formula I, can be prepared without undue experimentation by conventional techniques well known to those skilled in the art or by processes analogous to those described in the Schemes and Examples herein using appropriate isotopically-enriched reagents and/or intermediates.

Unless expressly stated to the contrary in a particular context, any of the various cyclic ring and ring system variables or substituents described herein may be attached to the rest of the compound at any ring atom (i.e., any carbon atom or any heteroatom) provided that a stable compound results.

Unless expressly stated to the contrary, all ranges cited herein are inclusive. For example, a heteroaromatic ring described as containing from “1 to 4 heteroatoms” means the ring can contain 1, 2, 3 or 4 heteroatoms. It is also to be understood that any range cited herein includes within its scope all of the sub-ranges within that range. Thus, for example, a heterocyclic ring described as containing from “1 to 4 heteroatoms” is intended to include as aspects thereof, heterocyclic rings containing 2 to 4 heteroatoms, 3 or 4 heteroatoms, 1 to 3 heteroatoms, 2 or 3 heteroatoms, 1 or 2 heteroatoms, 1 heteroatom, 2 heteroatoms, 3 heteroatoms, and 4 heteroatoms. Similarly, C₁-C₆ when used with a chain, for example an alkyl chain, means that the chain can contain 1, 2, 3, 4, 5 or 6 carbon atoms. It also includes all ranges contained therein including C₁-C₅, C₁-C₄, C₁-C₃, C₁-C₂, C₂-C₆, C₃-C₆, C₄-C₆, C₅-C₆, and all other possible combinations.

A “stable” compound is a compound which can be prepared and isolated and whose structure and properties remain or can be caused to remain essentially unchanged for a period of time sufficient to allow use of the compound for the purposes described herein (e.g., therapeutic administration to a subject). The compounds of the present invention are limited to stable compounds embraced by Formulas I.

The term “compound” refers to the compound and, in certain embodiments, to the extent they are stable, any hydrate or solvate thereof. A hydrate is the compound complexed with water, and a solvate is the compound complexed with an organic solvent.

As indicated above, the compounds of the present invention can be employed in the form of pharmaceutically acceptable salts. Those skilled in the art will recognize those instances in which the compounds of the invention may form salts. The term “pharmaceutically acceptable salt” refers to a salt (including an inner salt such as a zwitterion) which possesses effectiveness similar to the parent compound and which is not biologically or otherwise undesirable (e.g., is neither toxic nor otherwise deleterious to the recipient thereof). Thus, an embodiment of the invention provides pharmaceutically acceptable salts of the compounds of the invention. The term “salt(s)”, as employed herein, denotes any of the following: acidic salts formed with inorganic and/or organic acids, as well as basic salts formed with inorganic and/or organic bases. Salts of compounds of the invention may be formed by methods known to those of ordinary skill in the art, for example, by reacting a compound of the invention with an amount of acid or base, such as an equivalent amount, in a medium such as one in which the salt precipitates or in aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (“mesylates”), naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

In addition, when a compound of the invention contains both a basic moiety, such as, but not limited to an aliphatic primary, secondary, tertiary or cyclic amine, an aromatic or heteroaryl amine, pyridine or imidazole, and an acidic moiety, such as, but not limited to tetrazole or carboxylic acid, zwitterions (“inner salts”) may be formed and are included within the terms “salt(s)” as used herein. It is understood that certain compounds of the invention may exist in zwitterionic form, having both anionic and cationic centers within the same compound and a net neutral charge. Such zwitterions are included within the invention.

The compounds of Formula I may exist as rapidly interconverting tautomers with different points of attachment of hydrogen accompanied by one or more double bond shifts. The individual tautomers as well as mixtures thereof are encompassed by the present invention. The ratio between the tautomeric forms will vary depending on the conditions. As is well known to one of ordinary skill in the art, such compounds may be drawn and named in different ways. For example, the following structures depicted below show different ways that an illustrative compound of the invention may be drawn:

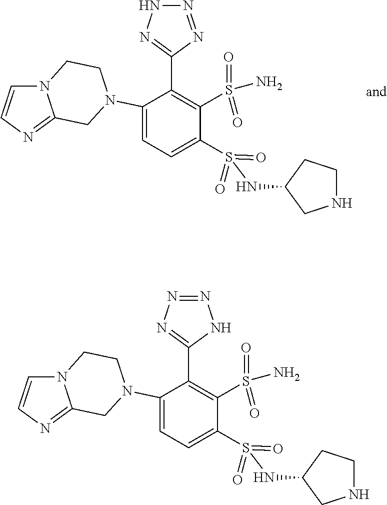 [SMILES: NS(=O)(=O)c1c(S(=O)(=O)N[C@@H]2CCNC2)ccc(N2CCn3ccnc3C2)c1-c1nn[nH]n1 ; NS(=O)(=O)c1c(S(=O)(=O)N[C@@H]2CCNC2)ccc(N2CCn3ccnc3C2)c1-c1nnn[nH]1]

It is understood that all possible tautomeric forms of the compounds of Formula I are contemplated as being within the scope of the instant invention, as well as mixtures thereof. It is further understood that while only one said tautomeric form of each example compound and embodiment of the invention may be depicted in the specification and appended claims, such depiction includes reference to all tautomeric forms of said compounds, which are included within the scope of the invention.

In the compounds of Formula I, ring Z is a tetrazolyl group, depicted as:

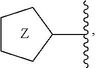

which is a 5-membered ring containing 4 nitrogen ring heteroatoms and one carbon atom, wherein Z is linked through a carbon to carbon bond to the phenyl core ring (see Formula I in the Summary of the Invention). As indicated above, all possible tautomeric forms of the compounds of Formula I are included within the scope of the invention. Thus, an indication of.

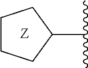

includes

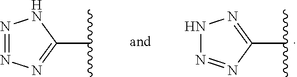 [SMILES: *c1nnn[nH]1 ; *c1nn[nH]n1]

As set forth above, the present invention includes pharmaceutical compositions comprising a compound of Formula I of the present invention, optionally one or more other active components (e.g., a β-lactam antibiotic), and a pharmaceutically acceptable carrier. The characteristics of the carrier will depend on the route of administration. By “pharmaceutically acceptable” is meant that the ingredients of the pharmaceutical composition must be compatible with each other, do not interfere with the effectiveness of the active ingredient(s), and are not deleterious (e.g., toxic) to the recipient thereof. Thus, compositions according to the invention may, in addition to the inhibitor, contain diluents, fillers, salts, buffers, stabilizers, solubilizers, and other materials well known in the art.

Also as set forth above, the present invention includes a method for treating a bacterial infection which comprises administering to a subject in need of such treatment a therapeutically effective amount of a compound of Formula I, or a pharmaceutically acceptable salt thereof, in combination with a β-lactam antibiotic and optionally a DHP inhibitor. The term “subject” (or, alternatively, “patient”) as used herein refers to an animal, preferably a mammal, and in particular a human or a non-human animal including livestock animals and domestic animals including, but not limited to, cattle, horses, sheep, swine, goats, rabbits, cats, dogs, and other mammals in need of treatment. In select embodiment, the subject is a human. In select embodiments, the subject has been the object of treatment, observation or experiment. The term “administration” and variants thereof (e.g., “administering” a compound) in reference to a compound of Formula I mean providing the compound, or a pharmaceutically acceptable salt thereof, to the individual in need of treatment. When a compound or a salt thereof is provided in combination with one or more other active agents (e.g., a carbapenem antibiotic or a DHP inhibitor or both), “administration” and its variants are each understood to include provision of the compound or its salt and the other agents at the same time or at different times. When the agents of a combination are administered at the same time, they can be administered together in a single composition or they can be administered separately. It is understood that a “combination” of active agents can be a single composition containing all of the active agents or multiple compositions each containing one or more of the active agents. In the case of two active agents a combination can be either a single composition comprising both agents or two separate compositions each comprising one of the agents; in the case of three active agents a combination can be either a single composition comprising all three agents, three separate compositions each comprising one of the agents, or two compositions one of which comprises two of the agents and the other comprises the third agent; and so forth.

The compositions and combinations of the present invention are suitably administered in effective amounts. The term “effective amount,” when used with a β-lactamase inhibitor (including a DHP inhibitor), means the amount of active compound sufficient to inhibit β-lactamase and thereby elicit the response being sought (i.e., an “inhibition effective amount”) in a cell, tissue, system, animal or human. In one embodiment, the effective amount is a “therapeutically effective amount” for the alleviation of the symptoms of the disease or condition being treated (e.g., the healing of conditions associated with bacterial infection, and/or bacterial drug resistance) in combination with a β-lactam antibiotic. In another embodiment, the effective amount is a “prophylactically effective amount” for prophylaxis of the symptoms of the disease or condition being prevented. When the active compound (i.e., active ingredient) is administered as the salt, references to the amount of active ingredient are to the free acid or free base form of the compound. An “effective amount” of a β-lactam antibiotic is an amount sufficient to alleviate the symptoms of the disease or condition being treated (e.g., the healing of conditions associated with bacterial infection, and/or bacterial drug resistance).

The administration of a composition of the present invention is suitably parenteral, oral, sublingual, transdermal, topical, intranasal, intratracheal, intraocular, or intrarectal, wherein the composition is suitably formulated for administration by the selected route using formulation methods well known in the art, including, for example, the methods for preparing and administering formulations described in chapters 39, 41, 42, 44 and 45 in Remington—The Science and Practice of Pharmacy, 21^{st} edition, 2006. In one embodiment, compounds of the invention are administered intravenously in a hospital setting. In another embodiment, administration is oral in the form of a tablet or capsule or the like. When administered systemically, a therapeutic composition is for example, suitably administered at a sufficient dosage to attain a blood level of inhibitor of at least about 1 μg/mL, and in additional embodiment at least about 10 μg/mL, and at least about 25 μg/mL. For localized administration, much lower concentrations than this may be effective, and much higher concentrations may be tolerated.

Intravenous administration of a compound of the invention can be conducted by reconstituting a powdered form of the compound with an acceptable solvent. Suitable solvents include, for example, saline solutions (e.g., 0.9% Sodium Chloride Injection) and sterile water (e.g., Sterile Water for Injection, Bacteriostatic Water for Injection with methylparaben and propylparaben, or Bacteriostatic Water for Injection with 0.9% benzyl alcohol). The powdered form of the compound can be obtained by gamma-irradiation of the compound or by lyophilization of a solution of the compound, after which the powder can be stored (e.g., in a sealed vial) at or below room temperature until it is reconstituted. The concentration of the compound in the reconstituted IV solution can be, for example, in a range of from about 0.1 mg/mL to about 20 mg/mL.

The present invention also includes a method for inhibiting bacterial growth which comprises administering to a bacterial cell culture, or to a bacterially infected cell culture, tissue, or organism, an inhibition effective amount of a compound of Formula I. Additional embodiments of the invention include the bacterial growth inhibiting method just described, wherein the compound of the present invention employed therein is a compound of one of the embodiments, sub-embodiments or classes described above. The compound may optionally be used in the form of a pharmaceutically acceptable salt in these embodiments. The method can involve administration of a compound of Formula I to an experimental cell culture in vitro to prevent the growth of β-lactam resistant bacteria. The method can alternatively involve administration of a compound of Formula I to an animal, including a human, to prevent the growth of β-lactam resistant bacteria in vivo. In these cases, the compound of Formula I is typically co-administered with a β-lactam antibiotic.

Compounds of the invention can be employed for the treatment, prophylaxis or inhibition of bacterial growth or infections due to bacteria that are resistant to β-lactam antibiotics in combination with a β-lactam antibiotic. More particularly, the bacteria can be metallo-β-lactamase positive strains that are highly resistant to β-lactam antibiotics. The terms “slightly resistant” and “highly resistant” are well-understood by those of ordinary skill in the art (see, e.g., Payne et al., Antimicrobial Agents and Chemotherapy 38:767-772 (1994); Hanaki et al., Antimicrobial Agents and Chemotherapy 30:11.20-11.26 (1995)). For the purposes of this invention, bacterial strains which are highly resistant to imipenem are those against which the MIC of imipenem is >16 μg/mL, and bacterial strains which are slightly resistant to imipenem are those against which the MIC of imipenem is >4 μg/mL.

Compounds of the invention can be used in combination with antibiotic agents for the treatment of infections caused by Class B-β-lactamase producing strains, in addition to those infections which are subsumed within the antibacterial spectrum of the antibiotic agent. Examples of class B-metallo-β-lactamase producing bacteria are Pseudomonas aeruginosa, Pseudomonas putida, Enterobacter cloacae, Klebsiella pneumoniae, Klebsiella oxytoca, Escherichia coli, Serratia marcescens, Enterobacter aerogenes, Enterobacter asburiae, Citrobacter freundii, Proteus mirabilis, Morganella morganii, Providencia rettgeri, and Acinetobacter baumannii.

It is generally advantageous to use a compound of Formula I in admixture or conjunction with a carbapenem, penicillin, cephalosporin, or other β-lactam antibiotic, or a prodrug thereof. It is advantageous to use a compound of Formula I in combination with one or more β-lactam antibiotics because of the class B β-lactamase inhibitory properties of the compounds. It is also advantageous to use a compound of Formula I in combination with one or more Class A, C, and D β-lactamase inhibitors to further limit β-lactam susceptibility. As already noted, the compound of Formula I and the β-lactam antibiotic can be administered separately (at the same time or as different times) or in the form of a single composition containing both active ingredients.

Carbapenems, penicillins, cephalosporins and other β-lactam antibiotics suitable for use in the present invention include both those known to show instability to or to be otherwise susceptible to class B-β-lactamases.

When the compounds of Formula I are combined with a carbapenem antibiotic, a dehydropeptidase (DHP) inhibitor can also be combined. Many carbapenems are susceptible to attack by a renal enzyme known as DHP. This attack or degradation may reduce the efficacy of the carbapenem antibacterial agent. Inhibitors of DHP and their use with carbapenems are disclosed in, e.g., U.S. Pat. Nos. 4,539,208; 4,616,038; 4,880,793; and 5,071,843. A preferred DHP inhibitor is 7-(L-2-amino-2-carboxyethylthio)-2-(2,2-dimethylcyclopropanecarboxamide)-2-heptenoic acid or a pharmaceutically acceptable salt thereof.

Carbapenems suitable for co-administration with compounds of the present invention include imipenem, ertapenem, meropenem, biapenem, (4R, 5S, 6S)-3-[3S, 5S)-5-(3-carboxyphenyl-carbamoyl)pyrrolidin-3-ylthio]-6-(1R)-1-hydroxyethyl]-4-methyl-7-oxo-1-azabicyclo[3.2.0]hept-2-ene-2-carboxylic acid, (1S, 5R, 6S)-2-(4-(2-(((carbamoylmethyl)-1,4-diazoniabicyclo[2.2.2]oct-1-yl)-ethyl(1,8-naphthosultam)methyl)-6-[1(R)-hydroxyethyl]-1-methylcarbapen-2-em-3-carboxylate chloride, BMS181139 ([4R-[4α,5β,6β(R*)]]-4-[2-[(aminoiminomethyl)amino]ethyl]-3-[(2-cyanoethyl)thio]-6-(1-hydroxyethyl)-7-oxo-1-azabicyclo[3.2.0]hept-2-ene-2-carboxylic acid), B02727 ([4R-3[3S*,5S*(R*)], 4α,5β,6β(R*)]]-6-(1-hydroxyethyl)-3-[[5-[1-hydroxy-3-(methylamino)propyl]-3-pyrrolidinyl]thio]-4-methyl-7-oxo-1-azabicyclo[3.2.0] hept-2-ene-2-carboxylic acid monohydrochloride), E1010 ((1R, 5S, 6S)-6-[1(R)-hydroxymethyl]-2-[2(S)-[1(R)-hydroxy-1-[pyrrolidin-3(R)-yl] methyl]pyrrolidin-4(S)-ylsulfanyl]-1-methyl-1-carba-2-penem-3-carboxylic acid hydrochloride) and S4661 ((1R,5S,6S)-2-[(3S,5S)-5-(sulfamoylaminomethyl) pyrrolidin-3-yl]thio-6-[(1R)-1-hydroxyethyl]-1-methylcarbapen-2-em-3-carboxylic acid), (1S,5R,6S)-1-methyl-2-{7-[4-(aminocarbonylmethyl)-1,4-diazoniabicyclo(2.2.2)octan-1yl]-methyl-fluoren-9-on-3-yl}-6-(1R-hydroxyethyl)-carbapen-2-em-3 carboxylate chloride.

Penicillins suitable for co-administration with compounds of the present invention include benzylpenicillin, phenoxymethylpenicillin, carbenicillin, azidocillin, propicillin, ampicillin, amoxicillin, epicillin, ticarcillin, cyclacillin, pirbenicillin, azlocillin, mezlocillin, sulbenicillin, piperacillin, and other known penicillins. The penicillins may be used in the form of pro-drugs thereof; for example as in vivo hydrolysable esters, for example the acetoxymethyl, pivaloyloxymethyl, α-ethoxy carbonyloxy-ethyl and phthalidyl esters of ampicillin, benzylpenicillin and amoxicillin; as aldehyde or ketone adducts of penicillins containing a 6-α-aminoacetamido side chain (for example hetacillin, metampicillin and analogous derivatives of amoxicillin); and as esters of carbenicillin and ticarcillin, for example the phenyl and indanyl α-esters.

Cephalosporins suitable for co-administration with compound of the present invention include cefatrizine, cephaloridine, cephalothin, cefazolin, cephalexin, cephacetrile, cephapirin, cephamandole nafate, cephradine, 4-hydroxycephalexin, cephaloglycin, cefoperazone, cefsulodin, ceftolozane, ceftazidime, cefuroxime, cefmetazole, cefotaxime, ceftriaxone, cefipime, and other known cephalosporins, all of which may be used in the form of pro-drugs thereof.

β-Lactam antibiotics other than penicillins and cephalosporins that may be co-administered with compounds of the present invention include aztreonam, latamoxef (MOXALACTAM), and other known β-lactam antibiotics such as carbapenems like imipenem, ertapenem, meropenem or (4R, 5S, 6S)-3-[(3S,5S)-5-(3-carboxyphenylcarbamoyl)pyrrolidin-3-ylthio]-6-(1R)-1-hydroxyethyl]-4-methyl-7-oxo-1-azabicyclo[3.2.0]hept-2-ene-2-carboxylic acid, all of which may be used in the form of pro-drugs thereof.

In one embodiment, the antibiotic co-administered with a compound of the present invention is selected from the group consisting of imipenem, ertapenem, meropenem and (4R, 5S, 6S)-3-[(3S,5S)-5-(3-carboxyphenylcarbamoyl)pyrrolidin-3-ylthio]-6-(R)-1-hydroxyethyl]-4-methyl-7-oxo-1-azabicyclo[3.2.0]hept-2-ene-2-carboxylic acid.

In another embodiment, the antibiotic co-administered with a compound of the present invention is selected from the group of penicillins consisting of ampicillin, amoxicillin, carbenicillin, piperacillin, azlocillin, mezlocillin, and ticarcillin. Such penicillins can optionally be used in the form of their pharmaceutically acceptable salts, for example their sodium salts. Ampicillin or amoxicillin can alternatively be employed in the form of fine particles of the zwitterionic form (generally as ampicillin trihydrate or amoxicillin trihydrate) for use in an injectable or infusable suspension. In an aspect of this embodiment, the penicillin co-administered with a compound of the present invention is amoxicillin, optionally in the form of its sodium salt or the trihydrate.

In another embodiment, the antibiotic co-administered with a compound of the present invention is selected from the group of cephalosporins consisting of cefotaxime, ceftriaxone, cefipime, and ceftazidime, which are optionally used in the form of their pharmaceutically acceptable salts, for example their sodium salts.

In certain embodiments of the invention, the compounds of the invention in combination with serine β-lactamase inhibitors (which can inhibit class A, C, D beta lactamases) in addition to β-lactam antibiotics. Serine β-lactamase inhibitors include but are not limited to avibactam, vaborbactam, relebactam, tazobactam, and clavulanic acid.

When co-administered with a β-lactam antibiotic, and optionally a β-lactamase inhibitor, the combination of the compound of the invention and the antibiotic can provide a synergistic effect. The terms “synergistic effect” and “synergy” indicate that the effect produced when two or more drugs are co-administered is greater than would be predicted based on the effect produced when the compounds are administered individually. While not wishing to be bound by theory, it is believed that the compounds of the present invention are β-lactamase inhibitors that act to prevent degradation of β-lactam antibiotics, thereby enhancing their efficacy and producing a synergistic effect.

Abbreviations employed herein include the following: Ac=acetyl=CH₃C(═O); ACN=MeCN=acetonitrile; aq=aqueous; BINAP=(2,2′-bis(diphenylphosphino)-1,1′-binaphthyl); BLI=β-lactamase inhibitor; Bn=benzyl; BOC (or Boc)=tert-butyloxycarbonyl; Boc anhydride=Boc₂O=di-tert-butyl dicarbonate; calc'd=calculated; CH₃CN=acetonitrile; CELITE=diatomaceous earth; conc.=concentrated; CV=column volume; DCM=dichloromethane; DIEA=N,N-Diisopropylethylamine; DMAP=4-dimethylaminopyridine or N,N-dimethylaminopyridine; DME=1,2-dimethoxyethane; DMF=N,N-dimethylformamide; DMSO=dimethyl sulfoxide; DPPF-Pd-G3=methanesulfonato 1,1-ferrocenediyl-bis(diphenylphosphino) (2′-amino-1,1′-biphenyl-2-yl) palladium(II); EA=AcOEt=EtOAc=ethyl acetate; Et=ethyl; EtOH=ethanol; HPLC=high-performance liquid chromatography; h or hr or hrs=hours; i-Pr=isopropyl alcohol; KOAc=potassium acetate; LCMS=LC-MS=liquid chromatography/mass spectrometry; LDA=lithium diisopropylamide; mCPBA=meta-chloroperoxybenzoic acid; Me=methyl; MeCN=acetonitrile; MeOH=methanol; MIC=minimum inhibitory concentration; min or mins=minutes; MTBE=methyl-t-butylether n-BuLi=n-butyllithium; NCS=N-Chlorosuccinimide; NMP=N-Methyl-2-pyrrolidone; NMR=nuclear magnetic resonance; Pd₂(dba)₃=Tris(dibenzylideneacetone)dipalladium, Pd(dppf)Cl₂=[1,1′-bis(diphenylphosphino)ferrocene]dichloropalladium(II); PE=Pet. ether=petroleum ether; Ph=phenyl; PMB=p-Methoxybenzyl; prep-HPLC=preparative HPLC; RAC-BINAP-PD-G3=methanesulfonato[2,2′-bis(diphenylphosphino)-1,1′-binapthyl](2′-amino-1,1′-biphenyl-2-yl)palladium(II); RBF=round bottom flask; RT=room temp.=room temperature; sat'd=saturated; SM=starting material; TBAF=tetrabutylammonium fluoride; TBS=tert-butyldimethylsilyl; TEA=triethylamine; TFA=trifluoroacetic acid; THF=tetrahydrofuran; TMS=trimethylsilane; X-Phos Pd G2=chloro(2-dicyclohexylphosphino-2′,4′,6′-triisopropyl-1,1′-biphenyl)[2-(2′-amino-1,1′-biphenyl)]palladium(II).

The compounds of the present invention can be readily prepared according to the following reaction schemes and examples, or modifications thereof, using readily available starting materials, reagents and conventional synthesis procedures. In these reactions, it is also possible to make use of known variants. Other methods for preparing compounds of the invention will be readily apparent to the person of ordinary skill in the art in light of the following reaction schemes and examples.

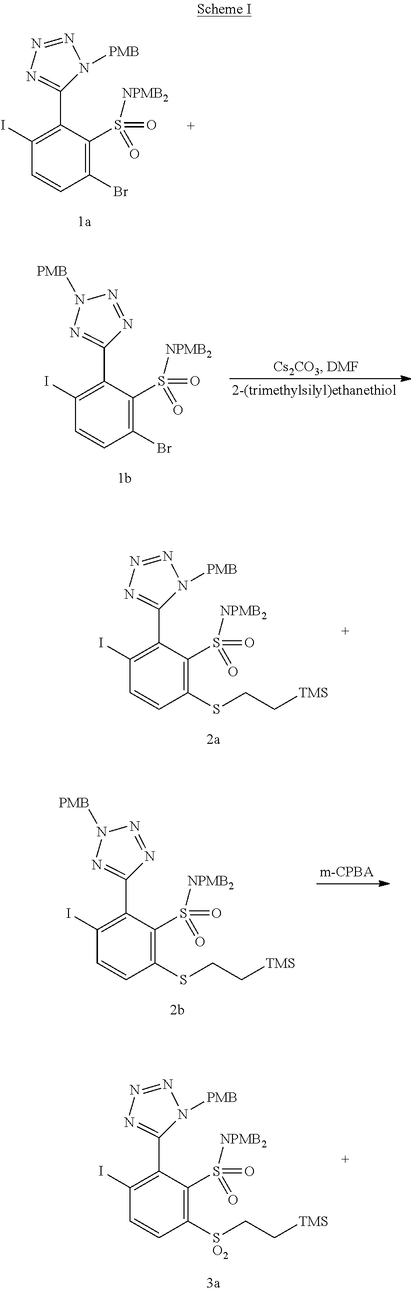

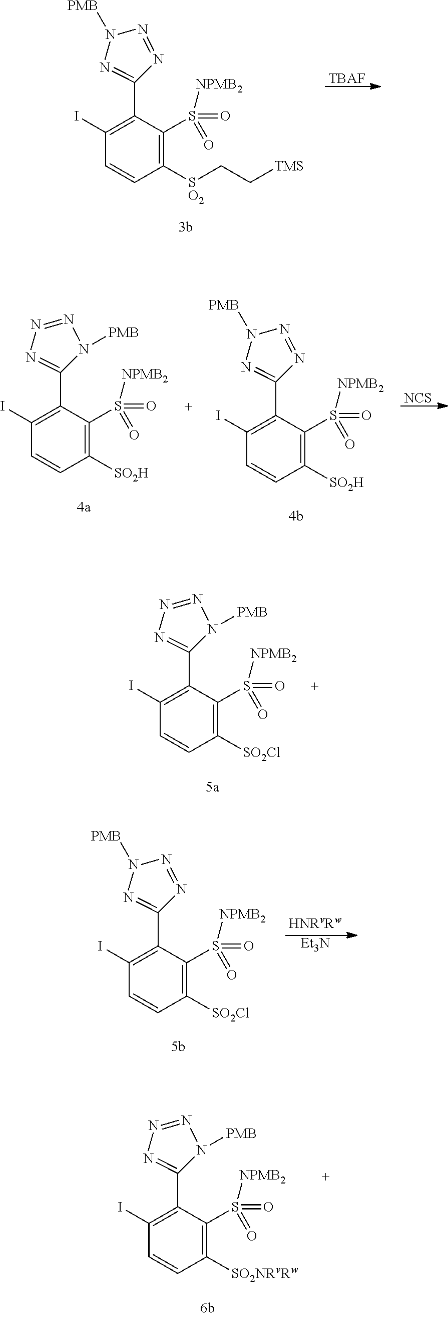

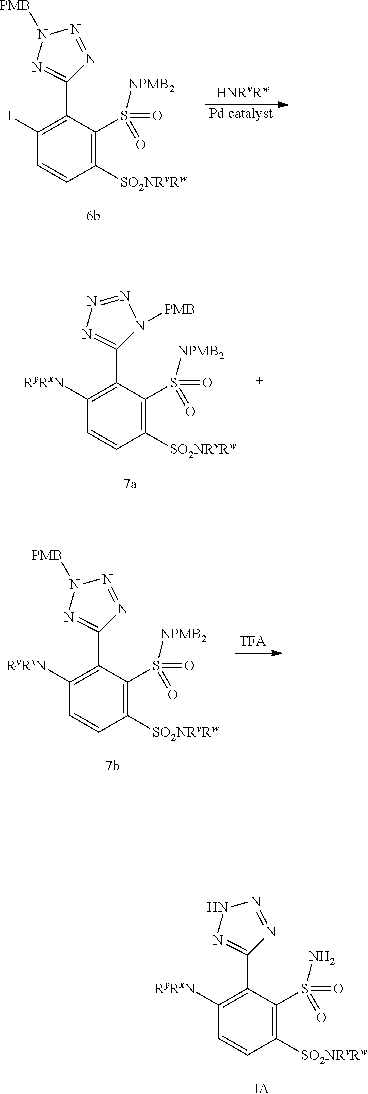

Sulfonamide compounds of the current invention, IA, may be prepared according to general Scheme I. According to the Scheme, bromide intermediates 1a and 1b (Scheme V) may be selectively reacted at the bromo position with 2-(trimethylsilyl)ethanethiol in the presence of a base (such as cesium carbonate) to afford sulfides 2a and 2b. Oxidation, for example, by using meta-chloroperoxybenzoic acid, gives sulfones 3a and 3b. Treatment with tetrabutylammonium fluoride (TBAF) gives the corresponding sulfinic acids 4a and 4b. The sulfinic acids may be converted to the corresponding sulfonyl chlorides 5a and 5b in a variety of ways, for example by treatment with N-chlorosuccinimide. Treatment of the sulfonyl chlorides 5a and 5b with a substituted or unsubstituted cyclic or acyclic amine (R^{v} and R^{w} can be independent substituents or combined or form a saturated heterocyclic ring) in the presence of a base such as triethylamine affords the sulfonamides 6a and 6b. Alternatively, sulfinic acids 4a and 4b may be directly converted in one pot to the sulfonamides 6a and 6b by reaction with N-chlorosuccinimide in the presence of the above-described amine reactant. Metal mediated C—N coupling, for example using palladium catalysts, using a variety of nitrogen containing cyclic or acyclic saturated reagents (R^{x} and R^{y} form a fully or partially saturated heterocyclic ring) afford analogs 7a and 7b. Final PMB protective group removal can be achieved under acidic conditions such as by using TFA in the optional presence of a carbocation scavenger, such as anisole or triethylsilane, providing target compounds IA.

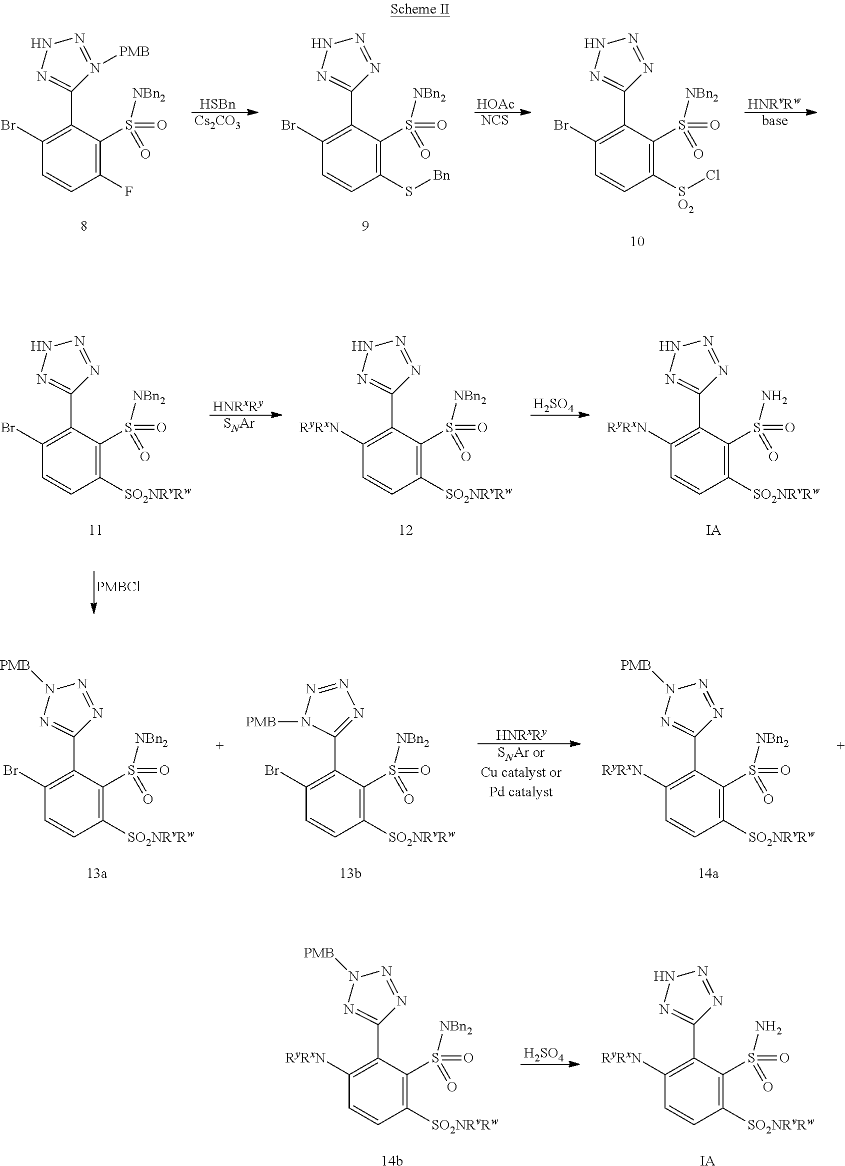

Alternatively as highlighted in Scheme II, fluoride intermediate 8 (Scheme VI) can be treated with a sulfide such as benzyl mercaptan in the presence of a base such as cesium carbonate to provide sulfide 9. Treatment of 9 with an oxidant such as N-chlorosuccinimide under acidic conditions will provide sulfonyl chloride 10. Treatment with an amine HNR^{x}R^{y} (see Scheme I) in the presence of a base will provide sulfonamide 11. Treatment with various nitrogen-containing cyclic partially or fully saturated reagents will provide intermediate 12. Finally cleavage of the benzyl groups can be accomplished after treatment with an appropriate acid such as sulfuric acid to provide target compounds IA. Alternatively the tetrazole of 11 can be protected with an appropriate protecting group such as para-methoxybenzyl to provide 13a and 13b. Subsequent metal mediated C—N coupling, for example using palladium catalysts, or nucleophilic aromatic substitution using a variety of nitrogen-containing cyclic or acyclic saturated reagents will afford intermediates 14a and 14b. Final cleavage of the PMB and benzyl protecting groups can be achieved under acidic conditions such as by using sulfuric acid to provide target compounds IA.

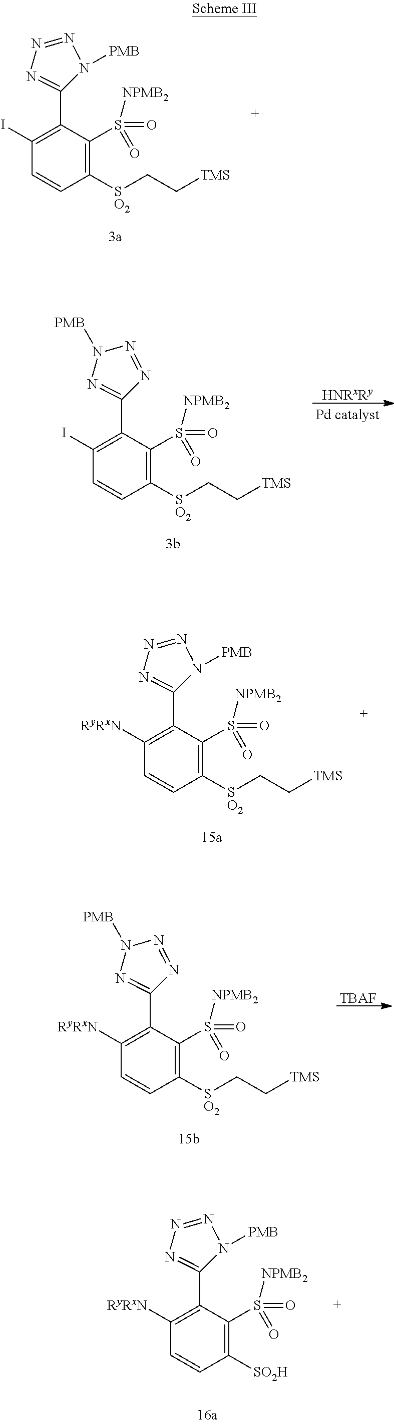

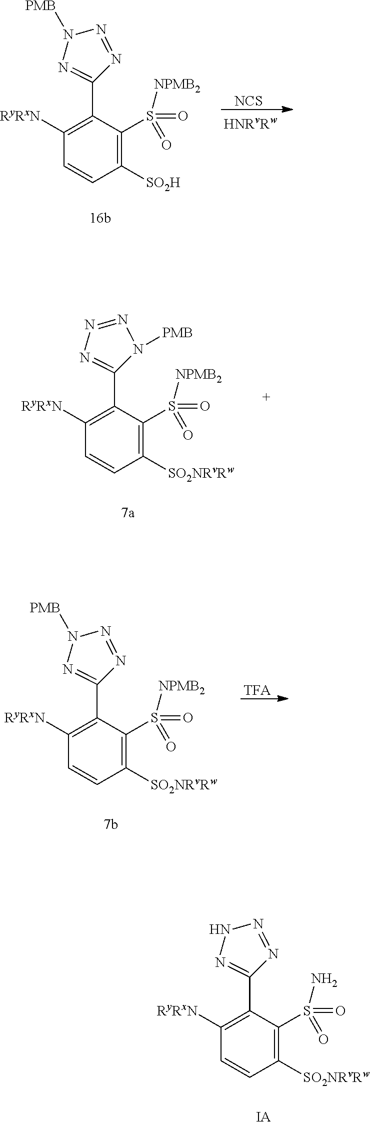

Alternatively, as highlighted in Scheme III, the sequence of steps to provide target compounds IA can be modified. For example, aryl iodides 3a and 3b can be treated with various nitrogen containing cyclic partially or fully saturated reagents under metal mediated C—N coupling or nucleophilic aromatic substitution conditions to provide intermediates 15a and 15b. Subsequent cleavage of the trimethylsilyl ethyl group with an appropriate reagent such as TBAF will provide sulfinic acids 16a and 16b. Conversion of the sulfinic acids to the sulfonyl chlorides, for example using NCS in the presence of various amines, will provide sulfonamides 7a and 7b. Finally cleavage of the protecting groups with an appropriate reagent such as TFA will provide target compounds IA.

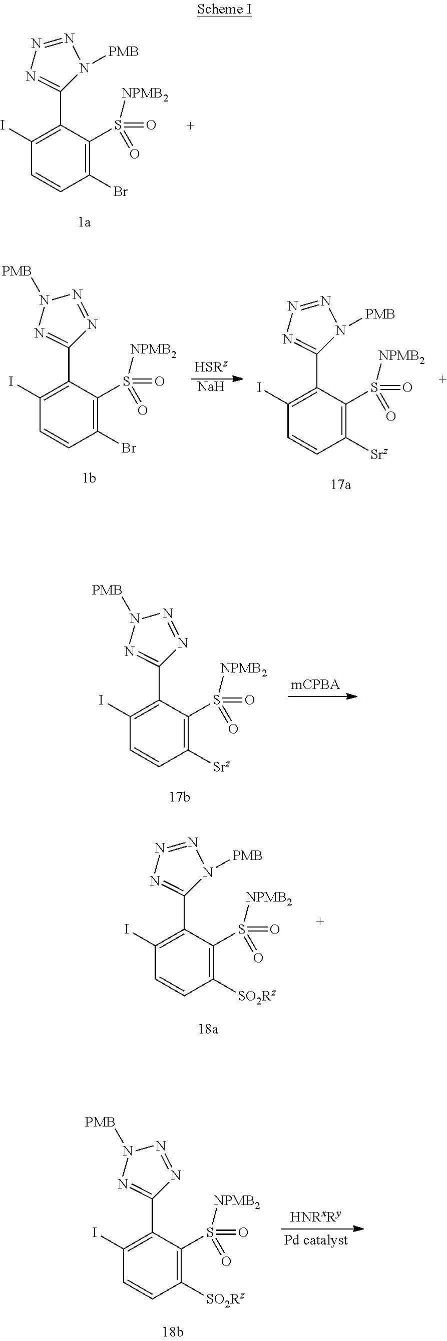

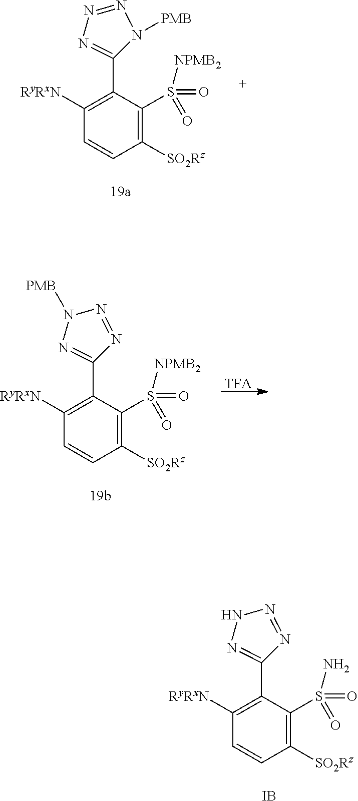

Sulfone compounds of the current invention, IB, may be prepared according to general Scheme IV. Bromides 1a and 1b (Scheme V) can be treated with various sulfides (where R^{z} can be a substituted or unsubstituted saturated heterocycle or saturated acycle) in the presence of a base such as sodium hydride to afford sulfides 17a and 17b. Oxidation of the sulfides to sulfones 18a and 18b can be accomplished using an oxidant such as meta-chloroperoxybenzoic acid. Subsequent metal mediated C—N coupling, for example using palladium catalysts, or nucleophilic aromatic substitution using a variety of nitrogen-containing cyclic partially or fully saturated reagents will afford intermediates 19a and 19b. Final cleavage of the PMB protecting groups can be achieved under acidic conditions such as by using trifluoroacetic acid to provide target compounds IB.

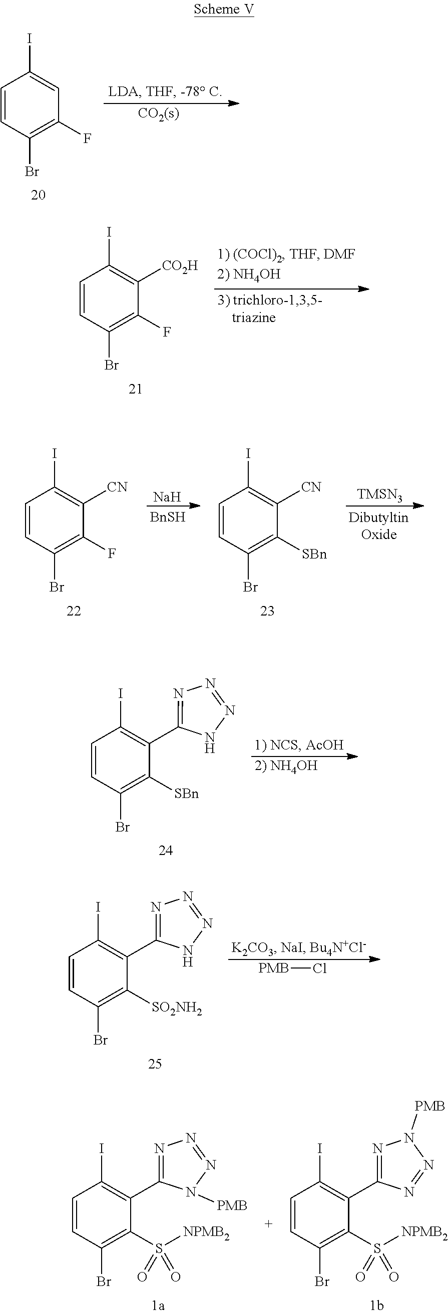

Intermediates 1a and 1b can be prepared according to Scheme V. According to the scheme, commercially available aryl fluoride 20 can be converted to the carboxylic acid 21 by treatment with LDA, followed by dry ice. The carboxylic acid functionality can be transformed to the corresponding nitrile 22 in numerous ways known in the art. One approach involves conversion to the acid chloride, for example using oxalyl chloride, followed by treatment with ammonium hydroxide to afford the carboxamide, and finally, dehydration, for example using trichloro-1,3,5-triazine, to give the nitrile 22. Nucleophilic aromatic substitution of the fluoride using benzyl mercaptan and a base such as sodium hydride provides the sulfide 23. The nitrile present in 23 can be converted to the tetrazole 24 using one of several methods, for example, by treatment with trimethylsilyl azide and dibutyltin oxide. Conversion of the benzyl sulfide to the sulfonyl chloride can be accomplished in several ways, for example, by treatment with N-chloro succinimide in acetic acid. Treatment with ammonium hydroxide then affords the sulfonamide 25. Concomitant protection of the tetrazole and sulfonamide to afford positional isomer mixture 1a and 1b can be achieved by treatment with excess of para-methoxybenzyl chloride in the presence of a base, such as potassium carbonate, and NaI and tetrabutyl ammonium chloride as catalysts. Typically 1a and 1b are used as a mixture of regioisomers, but the isomers can optionally be separated and used individually in the same way. In the examples below, it should be understood that the mixture of regioisomers or the individual regioisomers may be used interchangeably (occasionally only one isomer is shown for the sake of simplicity).

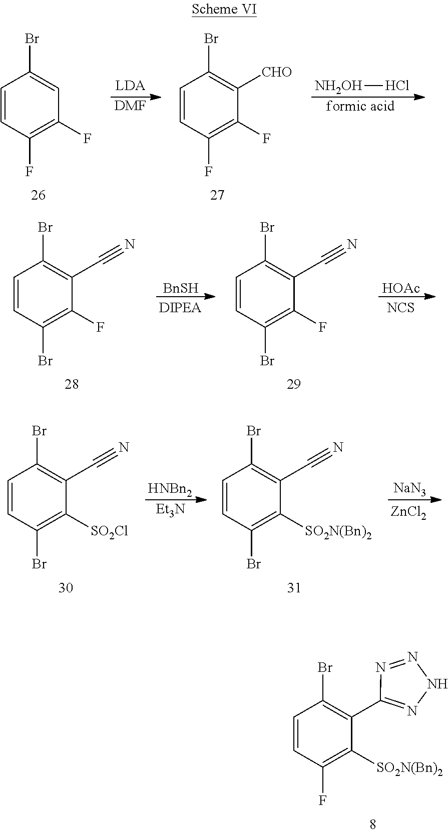

Intermediate 8 can be prepared according to Scheme VI. According to the scheme, commercially available aryl fluoride 26 can be converted to the aldehyde 27 by treatment with LDA, followed the addition of N,N-dimethylformamide. The aldehyde can then be converted to the nitrile 28 under various conditions including treatment with hydroxylamine hydrochloride and formic acid. Selective displacement of the fluoride ortho to the nitrile with benzyl mercaptan can be accomplished with various bases including N,N-diisopropylethylamine to provide the sulfide 29. The benzyl sulfide can be converted to the sulfonyl chloride 30 upon treatment with acetic acid in the presence of an oxidant such as N-chlorosuccinimide. Treatment with dibenzylamine in the presence of a base such as triethyl amine will provide sulfonamide 31. The nitrile of 31 can be converted to a tetrazole under various conditions including treatment with sodium azide in the presence of zinc chloride to afford 38.

Note that in the experimental procedures below, the REFERENCE EXAMPLES and EXAMPLES can be used as the mixture of para-methoxylbenzyl tetrazole regioisomers. Alternatively, the two regioisomers may be separated and each can be used as described below in the same fashion. In some REFERENCE EXAMPLES and EXAMPLES below, both regioisomers are explicitly used; however, in other cases, for the sake of simplicity, only one regioisomer was, in fact, typically used.

### Reference Example 1

### 6-bromo-3-iodo-N,N-bis(4-methoxybenzyl)-2-(1-(4-methoxybenzyl)-1H-tetrazol-5-yl)benzenesulfonamide and 6-bromo-3-iodo-N,N-bis(4-methoxybenzyl)-2-(2-(4-methoxybenzyl)-2H-tetrazol-5-yl)benzenesulfonamide

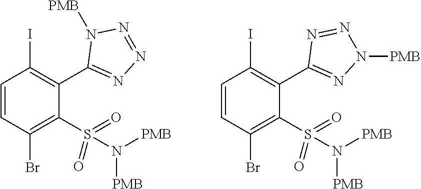

### Step A: 3-bromo-2-fluoro-6-iodobenzoic acid

Into a 5000-mL 4-necked round-bottom flask purged and maintained with an inert atmosphere of nitrogen, was placed bis(propan-2-yl)amine (121.2 g, 1.20 mol, 1.20 equiv), tetrahydrofuran (1000 mL). This was followed by the addition of butyllithium (440 mL, 1.10 equiv, 2.5 N) dropwise with stirring at −78° C. in 20 min. 60 min later, to this was added a solution of 1-bromo-2-fluoro-4-iodobenzene (300 g, 997 mmol, 1.00 equiv) in tetrahydrofuran (2000 mL) dropwise with stirring at −78° C. in 30 min. The resulting solution was stirred for 2 hr at −78° C. in a liquid nitrogen bath. The reaction progress was monitored by LCMS. The reaction was then quenched by pouring into 5000 g of dry ice. After stirring for 2 hours, the resulting mixture was concentrated under vacuum. The residue was dissolved in 3000 mL of 4N sodium hydroxide. The resulting solution was extracted with 2×1000 mL of ether and the aqueous layers combined. The pH value of the solution was adjusted to 2-3 with hydrogen chloride (1 mmol/L). The resulting solution was extracted with 4×1000 mL of ethyl acetate and the organic layers were combined and dried over anhydrous sodium sulfate and concentrated under vacuum. The crude product was purified by re-crystallization from hexanes.

### Step B: 3-bromo-2-fluoro-6-iodobenzoyl

Into a 5000-mL 3-necked round-bottom flask purged and maintained with an inert atmosphere of nitrogen, was placed 3-bromo-2-fluoro-6-iodobenzoic acid (273 g, 791.52 mmol, 1.00 equiv), tetrahydrofuran (2730 mL), and N,N-dimethylformamide (27.3 mL). This was followed by the addition of (COCl)₂ (110.9 g, 1.10 equiv) dropwise with stirring at 20° C. in 20 min. The resulting solution was stirred for 1 hr at room temperature. The reaction progress was monitored by LCMS. The resulting mixture was concentrated under vacuum.

### Step C: 3-bromo-2-fluoro-6-iodobenzamide

Into a 5000-mL 4-necked round-bottom flask purged and maintained with an inert atmosphere of nitrogen, was placed NH₄H (1200 g). This was followed by the addition of a solution of 3-bromo-2-fluoro-6-iodobenzoyl chloride (280 g, 771 mmol, 1.00 equiv) in tetrahydrofuran (2800 mL) dropwise with stirring at 0° C. in 30 min. The resulting solution was stirred for 1 hr at room temperature. The reaction progress was monitored by LCMS. The resulting mixture was concentrated under vacuum. The solids were collected by filtration, washed with H₂O to afford the title compound.

### Step D: 3-bromo-2-fluoro-6-iodobenzonitrile

Into a 10000-mL 4-necked round-bottom flask purged and maintained with an inert atmosphere of nitrogen, was placed 3-bromo-2-fluoro-6-iodobenzamide (270 g, 785.07 mmol, 1.00 equiv), N,N-dimethylformamide (5400 mL). This was followed by the addition of trichloro-1,3,5-triazine (1014 g, 5.50 mol, 7.00 equiv), in portions at 0° C. The resulting solution was stirred for 2 hr at room temperature. The reaction progress was monitored by LCMS. The reaction was then quenched by the addition of 15000 mL of sodium bicarbonate (aq). The solids were collected by filtration to afford the title compound.

### Step E: 2-(benzylsulfanyl)-3-bromo-6-iodobenzonitrile

Into a 5000-mL 4-necked round-bottom flask purged and maintained with an inert atmosphere of nitrogen, was placed sodium hydride (34 g, 852 mmol, 1.20 equiv, 60%), 1,4-dioxane (700 mL). This was followed by the addition of a solution of phenylmethanethiol (88.7 g, 714.15 mmol, 1.00 equiv) in 1,4-dioxane (950 mL) dropwise with stirring at 10° C. in 15 min. 30 min later, to this was added a solution of 3-bromo-2-fluoro-6-iodobenzonitrile (230 g, 705.73 mmol, 1.00 equiv) in 1,4-dioxane (1800 mL) dropwise with stirring at 10° C. The resulting solution was stirred for 2 hr at room temperature. The reaction progress was monitored by LCMS. The reaction was then quenched by pouring into 5000 mL of water/ice. The resulting solution was extracted with 5×1000 mL of ethyl acetate and the organic layers combined. The resulting mixture was washed with 2×1000 mL of water and 2×1000 mL of sodium bicarbonate and 2×1000 mL of sodium chloride. The mixture was dried over anhydrous sodium sulfate and concentrated under vacuum. The crude product was purified by re-crystallization from ether to afford the title compound.

### Step F: 5-[2-(benzylsulfanyl)-3-bromo-6-iodophenyl]-1H-1,2,3,4-tetrazole

Into a 2000-mL 4-necked round-bottom flask, was placed 2-(benzylsulfanyl)-3-bromo-6-iodobenzonitrile (66 g, 153.45 mmol, 1.00 equiv), toluene (660 mL), azidotrimethylsilane (44.2 g, 383.65 mmol, 2.50 equiv), dibutylstannanone (7.7 g, 30.93 mmol, 0.20 equiv). The resulting solution was stirred for 48 hr at 105° C. in an oil bath. The reaction progress was monitored by LCMS. The reaction mixture was cooled to room temperature. The resulting mixture was concentrated under vacuum. The residue was purified by silica gel column with tetrahydrofuran:PE (100:1) to afford the title compound.

### Step G: 6-bromo-3-iodo-2-(1H-1,2,3,4-tetrazol-5-yl)benzene-1-sulfonyl chloride

Into a 2000-mL 3-necked round-bottom flask purged and maintained with an inert atmosphere of nitrogen, was placed 5-[2-(benzylsulfanyl)-3-bromo-6-iodophenyl]-1H-1,2,3,4-tetrazole (115.6 g, 244.33 mmol, 1.00 equiv), acetic acid (1156 mL), water (115.6 mL), NCS (81.74 g, 612.15 mmol, 2.50 equiv). The resulting solution was stirred overnight at room temperature in an ice/salt bath. The reaction progress was monitored by LCMS. The resulting mixture was concentrated under vacuum to afford the title compound.

### Step H: 6-bromo-3-iodo-2-(1H-1,2,3,4-tetrazol-5-yl)benzene-1-sulfonamide

Into a 3000-mL 4-necked round-bottom flask purged and maintained with an inert atmosphere of nitrogen, was placed NH₄H (1180 mL), tetrahydrofuran (290 mL). This was followed by the addition of a solution of 6-bromo-3-iodo-2-(1H-1,2,3,4-tetrazol-5-yl)benzene-1-sulfonyl chloride (118 g, 262.54 mmol, 1.00 equiv) in tetrahydrofuran (300 mL) dropwise with stirring at 0° C. The resulting solution was stirred for 2 hr at 0-25° C. in an ice/salt bath (slowly warming to RT). The reaction progress was monitored by LCMS. The resulting mixture was concentrated under vacuum. The resulting solution was diluted with 500 mL of ether. After stirring for 30 min, the solids were collected by filtration to afford the title compound.

### Step I: 6-bromo-3-iodo-N,N-bis[(4-methoxyphenyl)methyl]-2-[1-[(4-methoxyphenyl)methyl]-1H-1,2,3,4-tetrazol-5-yl]benzene-1-sulfonamide and 6-bromo-3-iodo-N,N-bis[(4-methoxyphenyl)methyl]-2-[2-[(4-methoxyphenyl)methyl]-2H-1,2,3,4-tetrazol-5-yl]benzene-1-sulfonamide

Into a 3000-mL 4-necked round-bottom flask purged and maintained with an inert atmosphere of nitrogen, was placed 6-bromo-3-iodo-2-(1H-1,2,3,4-tetrazol-5-yl)benzene-1-sulfonamide (105 g, 244.17 mmol, 1.00 equiv), chloroform (1050 mL), potassium carbonate (168.9 g, 1.22 mol, 5.00 equiv), water (525 mL), NaI (11 g, 0.30 equiv), tetrabutyl(chloro)amine (20.4 g, 73.40 mmol, 0.30 equiv), 1-(chloromethyl)-4-methoxybenzene (230 g, 1.47 mol, 6.00 equiv). The resulting solution was stirred overnight at 50° C. in an oil bath. The reaction progress was monitored by LCMS. The reaction mixture was cooled to room temperature. The resulting solution was extracted with 2×1000 mL of dichloromethane and the organic layers combined and dried over anhydrous sodium sulfate and concentrated under vacuum to afford the title compounds.

LC-MS: (ES, m/z): 790 [M+H]⁺; H-NMR: (300 MHz, CDCl₃, ppm): δ 7.956-7.928 (m, 0.5H), 7.852-7.824 (m, 1H), 7.656-7.612 (m, 1.5H), 7.323-7.282 (m, 1.5H), 7.195-7.224 (m, 2H), 6.944-6.908 (m, 6H), 6.822-6.760 (m, 9H), 5.791 (m, 1H), 5.570-5.521 (m, 1H), 5.149-5.100 (m, 1H), 4.769-4.718 (m, 2H), 4.232-4.221 (m, 2H), 3.900-3.848 (m, 2H), 3.789-3.742 (m, 14H).

### Reference Example 2

### 3-iodo-N,N-bis(4-methoxybenzyl)-2-(2-(4-methoxybenzyl)-2H-tetrazol-5-yl)-6-((2-(trimethylsilyl)ethyl)sulfonyl)benzenesulfonamide and 3-iodo-N,N-bis(4-methoxybenzyl)-2-(1-(4-methoxybenzyl)-1H-tetrazol-5-yl)-6-((2-(trimethylsilyl)ethyl)sulfonyl)benzenesulfonamide

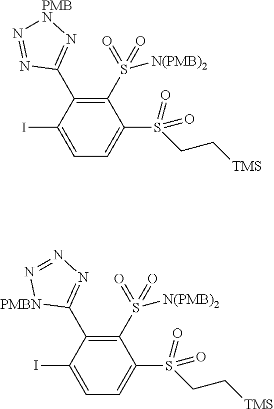

### Step A: 3-iodo-N,N-bis(4-methoxybenzyl)-2-(2-(4-methoxybenzyl)-2H-tetrazol-5-yl)-6-(2-(trimethylsilyl)ethylthio)benzenesulfonamide and 3-iodo-N,N-bis(4-methoxybenzyl)-2-(1-(4-methoxybenzyl)-1H-tetrazol-5-yl)-6-((2-(trimethylsilyl)ethyl)thio)benzenesulfonamide

6-bromo-3-iodo-N,N-bis[(4-methoxyphenyl)methyl]-2-[1-[(4-methoxyphenyl)methyl]-1H-1,2,3,4-tetrazol-5-yl]benzene-1-sulfonamide, 6-bromo-3-iodo-N,N-bis[(4-methoxyphenyl)methyl]-2-[2-[(4-methoxyphenyl)methyl]-2H-1,2,3,4-tetrazol-5-yl]benzene-1-sulfonamide (500 mg, 0.633 mmol), 2-(trimethylsilyl)ethanethiol (170 mg, 1.265 mmol) and Cs₂CO₃ (618 mg, 1.898 mmol) were combined in DMF (1.5 mL). Then the mixture was stirred at RT for 5 hr. The mixture was poured onto ether (100 mL) with the organic layer collected and concentrated under vacuum to give the title compounds: LCMS (ESI) calc'd for C₃₆H₄₂IN₅O₅S₂Si [M+H]⁺: 844, found 844; ¹H NMR (300 MHz, CDCl₃): δ 4.71-4.40 (m, 1H), 4.13-4.00 (m, 1H), 3.83-3.67 (m, 1H), 2.81-2.72 (m, 1H), 2.32-2.21 (m, 2H), 2.08-1.74 (m, 2H), 1.44 (s, 9H).

### Step B: 3-iodo-N,N-bis(4-methoxybenzyl)-2-(2-(4-methoxybenzyl)-2H-tetrazol-5-yl)-6-(2-(trimethylsilyl)ethylsulfonyl)benzenesulfonamide and 3-iodo-N,N-bis(4-methoxybenzyl)-2-(1-(4-methoxybenzyl)-1H-tetrazol-5-yl)-6-((2-(trimethylsilyl)ethyl)sulfonyl)benzenesulfonamide

3-iodo-N,N-bis(4-methoxybenzyl)-2-(2-(4-methoxybenzyl)-2H-tetrazol-5-yl)-6-((2-(trimethylsilyl)ethyl)thio)benzenesulfonamide (480 mg, 0.569 mmol) and m-CPBA (491 mg, 2.84 mmol) were combined in dichloromethane (2 mL). Then the mixture was stirred at RT for 4 hr. The resulting mixture was poured onto ether (200 mL). Then the mixture was washed with brine (150 mL). The organic layers were collected, dried over anhydrous Na₂SO₄ and concentrated under vacuum. The residue was then applied on a silica gel column with EA/PE (1/3) to give the title compounds: LCMS (ESI) calc'd for C₃₆H₄₂IN₅O₇S₂Si [M+H]⁺: 876, found 876; ¹H NMR (300 MHz, CDCl₃): δ 8.62 (d, J=8.7 Hz, 1H), 8.26 (d, J=8.4 Hz, 1H), 7.90-7.88 (m, 1H), 7.69-7.68 (m, 0.5H), 7.56-7.53 (m, 0.5H), 7.27-7.20 (m, 2H), 6.91-6.79 (m, 12H), 5.44-5.39 (m, 1H), 5.20-5.15 (m, 1H), 4.58-4.53 (m, 2H), 3.98-3.79 (m, 2H), 3.75-3.66 (m, 9H), 2.50-2.48 (m, 2H), 1.19-1.03 (m, 1H), 0.83-0.82 (m, 1H), 0.01 (s, 9H).

### Reference Example 3

### 2-(N,N-bis(4-methoxybenzyl)sulfamoyl)-4-iodo-3-(2-(4-methoxybenzyl)-2H-tetrazol-5-yl)benzenesulfinic acid and 2-(N,N-bis(4-methoxybenzyl)sulfamoyl)-4-iodo-3-(1-(4-methoxybenzyl)-1H-tetrazol-5-yl)benzenesulfinic acid

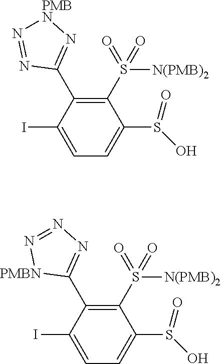

A solution of 3-iodo-N,N-bis(4-methoxybenzyl)-2-(2-(4-methoxybenzyl)-2H-tetrazol-5-yl)-6-((2-(trimethylsilyl)ethyl)sulfonyl)benzenesulfonamide (1.0 g, 1.14 mmol) in tetrahydrofuran (10 mL) was stirred with tetrabutylammonium fluoride (1.194 g, 4.57 mmol) at room temperature under N₂ for 0.5 hr. LCMS showed the desired mass of 776. The mixture was diluted with ethyl acetate, washed with saturated KHSO₄ aqueous, dried over MgSO₄, and concentrated under vacuum to provide the crude product. The crude material was used directly for the next step: LCMS (ESI) calc'd for C₃₁H₃₀IN₅O₇S₂ [M+H]⁺: 776, found 776; ¹H NMR (300 MHz, CDCl₃): δ 4.87-4.60 (bs, 1H), 4.36-4.21 (bs, 1H), 3.96-3.90 (m, 1H), 2.87-2.83 (m, 1H), 2.46-2.29 (m, 2H), 2.27 (d, J=1.2 Hz, 3H), 1.95-1.91 (m, 1H), 1.43 (s, 9H).

### Reference Example 4

### 2-(N,N-bis(4-methoxybenzyl)sulfamoyl)-4-iodo-3-(2-(4-methoxybenzyl)-2H-tetrazol-5-yl)benzenesulfonyl chloride and 2-(N,N-bis(4-methoxybenzyl)sulfamoyl)-4-iodo-3-(1-(4-methoxybenzyl)-1H-tetrazol-5-yl)benzenesulfonyl chloride

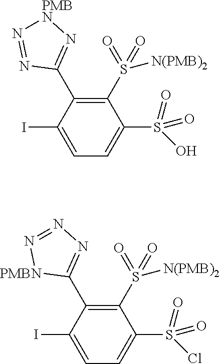

2-(N, N-bis(4-methoxybenzyl)sulfamoyl)-4-iodo-3-(2-(4-methoxybenzyl)-2H-tetrazol-5-yl)benzenesulfinic acid (800 mg, 1.031 mmol) in tetrahydrofuran (10 mL) was cooled to 0° C. 1-chloropyrrolidine-2,5-dione (275 mg, 2.063 mmol) in tetrahydrofuran (2 mL) was added over 5 min. The mixture was stirred at the same temperature for 30 min, and monitored by LCMS (a small amount SM is still shown), then diluted with ethyl acetate, washed with saturated NaHCO₃ and brine, dried over MgSO₄, and concentrated to get the crude product: LCMS (ESI) calc'd for C₃₁H₂₉ClIN₅O₇S₂ [M+H]⁺: 810, found 810.

### Reference Example 5

### tert-butyl (R)-3-((2-(N,N-bis(4-methoxy benzyl)sulfamoyl)-4-iodo-3-(1-(4-methoxy benzyl)-1H-tetrazol-5-yl)phenyl)sulfonamido)pyrrolidine-1-carboxylate and tert-butyl (R)-3-((2-(N,N-bis(4-methoxybenzyl)sulfamoyl)-4-iodo-3-(2-(4-methoxybenzyl)-2H-tetrazol-5-yl)phenyl)sulfonamido)pyrrolidine-1-carboxylate

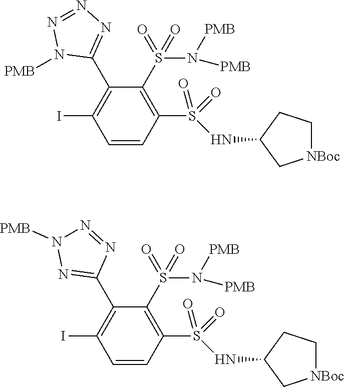

To a solution of a mixture of 2-(N,N-bis(4-methoxy benzyl)sulfamoyl)-4-iodo-3-(2-(4-methoxy benzyl)-2H-tetrazol-5-yl)benzene-1-sulfonyl chloride and 2-(N,N-bis(4-methoxybenzyl)sulfamoyl)-4-iodo-3-(2-(4-methoxybenzyl)-1H-tetrazol-5-yl)benzene-1-sulfonyl chloride (REFERENCE EXAMPLE 4) (0.92 g, 1.1 mmol) in tetrahydrofuran (11 mL) was added (R)-tert-butyl 3-aminopyrrolidine-1-carboxylate (423 mg, 2.3 mmol) at 0° C. The reaction was kept at 0° C. for 30 min. The mixture was diluted with EA and washed sequentially with 1 M aqueous HCl and brine, dried over MgSO₄ and filtered. The filtrate was concentrated under vacuum. The residue was purified by silica gel column chromatography (gradient elution 0 to MeOH in DCM) to give the title compound: LCMS (ESI) calc'd for C₄₀H₄₆IN₇O₉S₂: [M+1]⁺ 960 found 960; H NMR (400 MHz, DMSO-d₆) δ 8.52 (d, J=8.4 Hz, 1H), 8.29 (d, J=8.4 Hz, 1H), 7.29-7.25 (m, 2H), 6.83-6.69 (m, 10H), 5.95 (brs, 1H), 5.55-5.50 (m, 0.5H), 5.24-5.19 (m, 0.5H), 4.58-4.53 (m, 1H), 4.05-3.81 (m, 5H), 3.85 (s, 9H), 3.48-3.35 (m, 4H), 2.02-1.82 (m, 2H), 1.44 (s, 9H).

### Reference Example 6

### tert-butyl (R)-(3-((2-(N,N-bis(4-methoxybenzyl)sulfamoyl)-4-iodo-3-(2-(4-methoxybenzyl)-2H-tetrazol-5-yl)phenyl)sulfonamido)-2-hydroxypropyl)carbamate

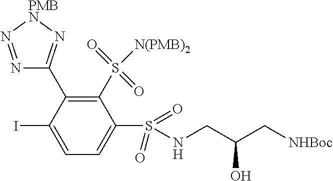

To a solution of 2-(N,N-bis(4-methoxybenzyl)sulfamoyl)-4-iodo-3-(2-(4-methoxybenzyl)-2H-tetrazol-5-yl)benzenesulfinic acid (38 g, 49.0 mmol) (REFERENCE EXAMPLE 3) in THF (300 mL) was added 1-chloropyrrolidine-2,5-dione (6.54 g, 49.0 mmol) at room temperature under nitrogen. The solution was stirred at room temperature for 1 h. After that time, to the reaction mixture were added (S)-tert-butyl (3-amino-2-hydroxypropyl)carbamate (13.98 g, 73.5 mmol) and triethylamine TEA (10.24 mL, 73.5 mmol). The mixture was stirred at room temperature for 30 min. The resultant mixture was filtered and concentrated under vacuum. The residue was purified by silica gel column chromatography, eluting with 50% EA in PE to afford (R)-tert-butyl (3-(2-(N,N-bis(4-methoxybenzyl)sulfamoyl)-4-iodo-3-(2-(4-methoxybenzyl)-2H-tetrazol-5-yl)phenylsulfonamido)-2-hydroxypropyl)carbamate. LC/MS [M+H]⁺. 964.4.

### Reference Example 7

### (R)-tert-butyl (3-(2-(N,N-bis(4-methoxybenzyl)sulfamoyl)-4-iodo-3-(2-(4-methoxybenzyl)-2H-tetrazol-5-yl)phenylsulfonamido)-2-((tert-butyldimethylsilyl)oxy)propyl)carbamate

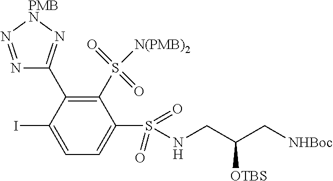

To a solution of (tert-butyl (R)-(3-((2-(N,N-bis(4-methoxybenzyl)sulfamoyl)-4-iodo-3-(2-(4-methoxybenzyl)-2H-tetrazol-5-yl)phenyl)sulfonamido)-2-hydroxypropyl)carbamate (REFERENCE EXAMPLE 6) (2.00 g, 2.07 mmol) in DMF (20 mL) were added 1H-imidazole (1.10 g, 16.60 mmol) and TBS-Cl (1.90 g, 12.45 mmol) at room temperature. The reaction solution was stirred at room temperature for 16 h. The resulting solution was diluted with water (50 mL), and then extracted with EA (3×40 mL). The organic layers were combined, washed with brine (2×30 mL), dried over anhydrous Na₂SO₄ and filtered. The filtrate was concentrated under vacuum. The residue was purified by a silica gel column chromatography, eluted with 50% EA in PE. The fractions containing desired product were combined and concentrated under vacuum to afford (R)-tert-butyl 3-(2-(N,N-bis(4-methoxybenzyl)sulfamoyl)-4-iodo-3-(2-(4-methoxybenzyl)-2H-tetrazol-5-yl)phenylsulfonamido)-2-((tert-butyldimethylsilyl)oxy)propyl)carbamate as a solid: LCMS (ESI) calc'd for C₄₅H₆₀IN₇O₁₀S₂Si [M+H]⁺: 1078, found 1078.

### Reference Example 8

### tert-butyl (R)-3-((4-bromo-2-(N,N-dibenzylsulfamoyl)-3-(2H-tetrazol-5-yl)phenyl)sulfonamido)pyrrolidine-1-carboxylate

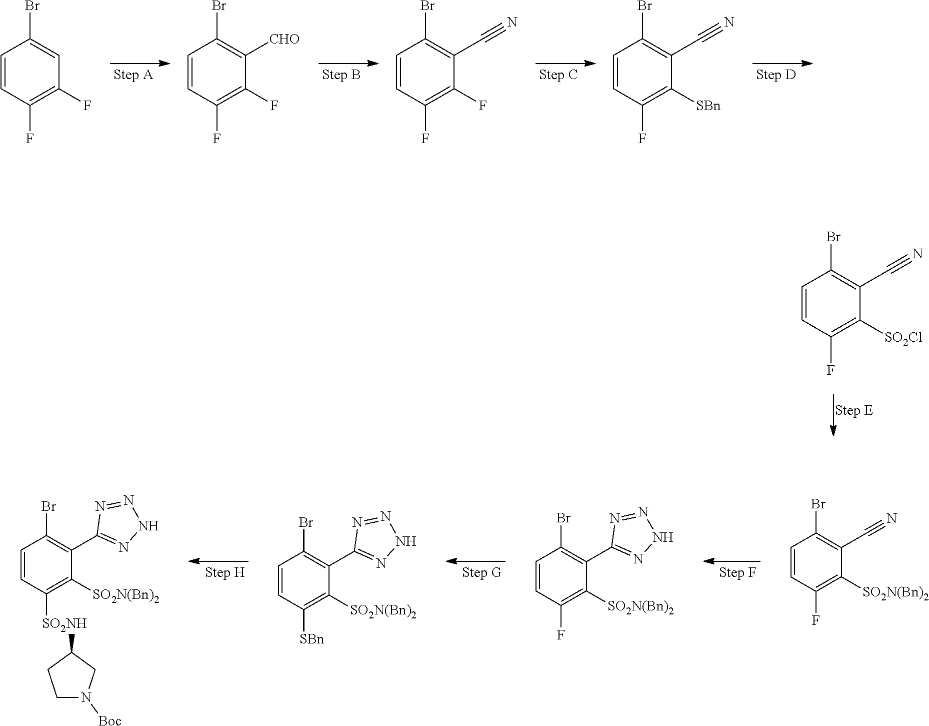

### Step A: 6-bromo-2,3-difluorobenzaldehyde

To a 2000 mL 3 N RBF with stir bar, PE dropping funnel and internal thermometer, under nitrogen sweep, was added 1-bromo-3,4-difluorobenzene (60 mL, 531 mmol) and 600 mL THF. The reaction was cooled to −70° C. and lithium diisopropylamide (318 mL, 637 mmol) was added dropwise over ˜1.5 hr while the temperature is maintained below −60° C. The reaction was then stirred for 15-20 min then DMF (49.3 mL, 637 mmol) was added dropwise over 10 min maintaining the temperature below −65° C. The mixture was stirred for an additional 30 min then removed cooling bath and warmed to −20° C. The reaction mixture was quenched by the addition of water (75 mL) followed by the addition of 5N HCl (˜400 mL) until ph 4 was achieved. The resultant mixture was stirred at RT and partitioned with 500 mL MTBE. The organic layer was washed with sat. NaHCO₃ (200 mL) and brine (100 mL), dried over Na₂SO₄, filtered and concentrated in vacuo. The crude product was adsorbed on silica (˜1 g/g of crude) and chromatographed (3 columns total, 330 g column, condition with hexanes, eluted 1 CV hexanes then gradient to 10% MTBE/hexanes over 4 CV then hold for 4 CV) to give 6-bromo-2,3-difluorobenzaldehyde.

### Step B: 6-bromo-2,3-difluorobenzonitrile

To a 1000 mL 4 N flask with stir bar, internal thermometer and air condenser were added 6-bromo-2,3-difluorobenzaldehyde (96.14 g, 435 mmol), hydroxylamine hydrochloride (33.3 g, 479 mmol) and formic acid (492 mL, 13.1 mol). The reaction was heated to 90-95° C. (internal temp) and the mixture was stirred for 18 hours. The reaction mixture was cooled to RT and poured into water (1400 mL) and stirred vigorously for 20 min. The suspended solids were collected by filtration and washed with water (1000 mL). The filtrate was diluted with an additional 500 mL water and refiltered to give additional product (further dilution not needed). The solids were dried using a combination of vacuum in addition to a flow of nitrogen for 18 hours to give 6-bromo-2,3-difluorobenzonitrile.

### Step C: 2-(benzylthio)-6-bromo-3-fluorobenzonitrile

To a 1000 mL flask with stir bar were added 6-bromo-2,3-difluorobenzonitrile (82.26 g, 377 mmol), N,N-diisopropylethylamine (99 mL, 566 mmol) and THF (400 mL). Neat benzyl mercaptan (44.9 mL, 383 mmol) was then added and the reaction was stirred at RT for 20 hours. The reaction was diluted with EtOAc (1200 mL), washed sequentially with water (250 mL), 5N HCl (2×120 mL), sat. NaHCO₃ (2×100 mL), brine (100 mL) then dried over Na₂SO₄, filtered and concentrated in vacuo. The crude product was suspended in 500 mL heptane and stirred vigorously for 30 min. The resultant solid was collected, washed with 200 mL heptane then dried using a under vacuum followed by a nitrogen stream to give the title compound.

### Step D: 3-bromo-2-cyano-6-fluorobenzene-1-sulfonyl chloride

To a 2000 mL RBF with stir bar were added 2-(benzylthio)-6-bromo-3-fluorobenzonitrile (108.22 g, 336 mmol) and acetic acid. The mixture was stirred until the solid dissolved. After that time, water (108 mL, 5995 mmol) was added followed by N-chlorosuccinimide (112 g, 840 mmol) and the reaction mixture was stirred at RT for 2 hrs. After that time, the reaction was poured into 2200 mL water and stirred vigorously for 20 min. The resultant solid was collected by filtration, washed with water (2×200 mL), slurried in hexanes and washed with hexanes (1×200 mL) and dried under a combination of vacuum and a flow of nitrogen to provide the title compound.

### Step E: N,N-dibenzyl-3-bromo-2-cyano-6-fluorobenzenesulfonamide

To a 1000 mL RBF with stir bar were added triethylamine (28.0 mL, 201 mmol), dibenzylamine (21.26 mL, 111 mmol) and CH₂Cl₂ (300 mL). The reaction mixture was cooled in an ice/water bath and add 3-bromo-2-cyano-6-fluorobenzene-1-sulfonyl chloride (30 g, 100 mmol) was added portionwise. The mixture was stirred for 10 min at 0° C. then an additional 30 min at RT. The reaction was then taken up in 750 mL of EtOAc and the organic layer was washed sequentially with 1M HCl (2×300 mL), sat NaHCO₃(2×150 mL), sat'd brine (100 mL), dried over Na₂SO₄, filtered and concentrated in vacuo. The crude solid was slurried in hexanes, filtered, washed with hexanes, then dried under a combination of vacuum and a stream of nitrogen stream to provide the title product.

### Step F: N,N-dibenzyl-3-bromo-6-fluoro-2-(2H-tetrazol-5-yl)benzenesulfonamide

To a 1000 mL 1N RBF with stir bar were added N,N-dibenzyl-3-bromo-2-cyano-6-fluorobenzenesulfonamide (40.5 g, 88 mmol), sodium azide (17.20 g, 265 mmol), zinc chloride (18.02 g, 132 mmol), toluene (304 mL) followed by N,N,N′,N′-tetramethylethylenediamine (21.15 mL, 141 mmol). The reaction was heated at 95° C. for 2 hours. The reaction was cooled to RT then 300 mL water was added followed by 300 mL hexanes. The resultant suspension was stirred for 1 hour. The suspension was filtered and the collected solid was washed with water (2×300 mL). To the wet solid were added 400 mL 1N HCl and 40 mL MeOH. The resultant mixture was stirred vigorously for 3 hours. The suspension was filtered, washed with 300 mL water and 200 mL hexanes then dried under a combination of house vacuum along with a nitrogen stream overnight to provide the title product.

### Step G: N,N-dibenzyl-6-(benzylthio)-3-bromo-2-(2H-tetrazol-5-yl)benzenesulfonamide

To a 40 mL vial with stir bar were added N,N-dibenzyl-3-bromo-6-fluoro-2-(2H-tetrazol-5-yl)benzenesulfonamide (3.24 g, 6.45 mmol), Cs₂CO₃ (5.25 g, 16.12 mmol), NMP (16.20 mL) followed by benzyl mercaptan (1.134 mL, 9.67 mmol). The mixture was stirred at ambient temperature for 1.5 hr. The reaction mixture was taken up in 200 mL EtOAc and the organic layer was washed sequentially with 1N HCl (2×50 mL), 10% LiCl (aq.) (2×50 mL) and brine (50 mL) then dried over Na₂SO₄, filtered and concentrated in vacuo. The crude product was purified by flash silica chromatography (column conditioned with hexanes, then product eluted 0.5 CV, then gradient to 100% EtOAc over 12 CV, then hold at 100% EtOAc for 6 CV) to give the title product: calc'd. m/z 605.1 and 607.1 [M], Found m/z 606.2 and 608.2 [M+H]⁺.

### Step H: (R)-tert-butyl 3-(4-bromo-2-(N,N-dibenzylsulfamoyl)-3-(2H-tetrazol-5-yl)phenylsulfonamido)pyrrolidine-1-carboxylate

To a 500 mL RBF containing N,N-dibenzyl-6-(benzylthio)-3-bromo-2-(2H-tetrazol-5-yl)benzenesulfonamide (22.67 g, 37.4 mmol) was added acetic acid (227 mL) followed by the addition of NCS (14.97 g, 112 mmol) and water (22.67 mL) and the mixture was stirred at RT for 45 min. The reaction mixture was poured into 500 mL water and stirred for 30 min. The product became a gum around the stir bar. The supernatant was decanted and additional 300 mL water was added to the flask, mixed and decanted. The resultant gum was taken up in MTBE (500 mL) washed with water (2×100 mL) and brine (2×100 mL), then dried over Na₂SO₄, filtered and concentrated in vacuo to provide the intermediate sulfonyl chloride. A solution of the sulfonyl chloride was added dropwise to a flask containing a cooled (0° C.) mixture of Et₃N (10.42 mL, 74.7 mmol) and (R)-1-BOC-3-aminopyrrolidine (6.97 mL, 41.1 mmol) in 100 mL of THF. The resultant mixture was stirred at 0° C. for 15 min, followed by an additional 15 min at RT. The reaction mixture was diluted with 500 mL EtOAc, then washed sequentially with 2×100 mL 1N HCl, 1×100 mL pH 7 buffer, 1×100 mL sat brine then dried over Na₂SO₄, filtered and concentrated in vacuo. The crude product was purified by flash silica chromatography (column conditioned with hexanes, loaded crude product in 30 mL DCM on to top of column, eluted 1 CV hexanes then gradient to 100% 3:1 EtOAc/EtOH over 6 CV then hold for 6 CV) to provide the title compound. ESI MS calc'd m/z 731.1 and 733.1 [M], Found m/z 732.4 and 734.4 [M+H]⁺.

### Reference Example 9

### tert-butyl (R)-3-((4-bromo-2-(N,N-dibenzylsulfamoyl)-3-(1-(4-methoxybenzyl)-1H-tetrazol-5-yl)phenyl)sulfonamido)pyrrolidine-1-carboxylate intermediate

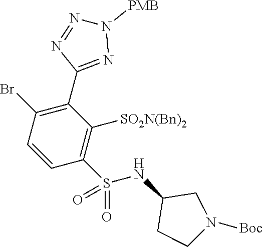

To a suspension of tert-butyl (R)-3-((4-bromo-2-(N,N-dibenzylsulfamoyl)-3-(2H-tetrazol-5-yl)phenyl)sulfonamido)pyrrolidine-1-carboxylate (3.05 g, 4.16 mmol) (REFERENCE EXAMPLE 8) in acetonitrile (41.6 mL) were added 4-methoxybenzyl chloride (0.734 mL, 5.41 mmol) and DIEA (1.818 mL, 10.41 mmol) and stirred at 25° C. for 16 hours. After that time, additional DIEA (1.818 mL, 10.41 mmol) and 4-methoxybenzyl chloride (0.734 mL, 5.41 mmol) was added and the reaction mixture was stirred at RT for an additional 12 hours. The reaction mixture was diluted with DCM 100 mL and washed with water 30 mL followed by brine 30 mL. The organic layer was dried over Na₂SO₄, filtered and concentrated in vacuo. The residue was purified by column chromatography on silica gel, eluting with a gradient of EtOAc in hexane 0 to 50% to afford the title compound.

### Reference Example 10

### (R)-tert-butyl (3-(4-bromo-2-(N,N-dibenzylsulfamoyl)-3-(2-(4-methoxybenzyl)-2H-tetrazol-5-yl)phenylsulfonamido)-2-((tert-butyldimethylsilyl)oxy)propyl)carbamate

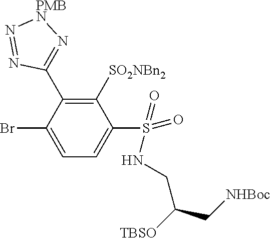

### Step A: N,N-dibenzyl-3-bromo-2-(2H-tetrazol-5-yl)-6-((2-(trimethylsilyl)ethyl)thio) benzenesulfonamide

To a mixture of NaH (10.75 g, 0.27 mol, 60% in mineral oil) in DMF (500 mL) was added 2-(trimethylsilyl)ethanethiol (30.10 g, 0.22 mol) at 0° C. under nitrogen. The mixture was stirred at 0° C. for 0.5 h under nitrogen. Then N,N-dibenzyl-3-bromo-6-fluoro-2-(2H-tetrazol-5-yl)benzenesulfonamide (Reference example 8 Step F) (45.0 g, 0.09 mol) was added slowly into the resulting mixture at room temperature. The reaction mixture was stirred at room temperature for 1 h. The resulting mixture was used in the next step directly without further purification: LCMS (ESI) calc'd for C₂₆H₃₀BrN₅O₂S₂Si [M+H]⁺: 616, 618, found 616, 618.

### Step B: N,N-dibenzyl-3-bromo-2-(2-(4-methoxybenzyl)-2H-tetrazol-5-yl)-6-((2-(trimethylsilyl)ethyl)thio)benzenesulfonamide

To resulting mixture above was added K₂CO₃ (37.0 g, 0.27 mol) at 0° C., and then it was added slowly 1-(chloromethyl)-4-methoxybenzene (56.0 g, 0.36 mol) at 0° C. The mixture was stirred at room temperature for 16 h. The resulting mixture was diluted with water (1 L), and then extracted with EA (3×500 mL). The organic layers were combined, washed with brine (2×300 mL), dried over anhydrous Na₂SO₄ and filtered. The filtrate was concentrated under vacuum. The residue was purified by a silica gel column chromatography, eluted with ethyl 40% EA in PE to afford N,N-dibenzyl-3-bromo-2-(2-(4-methoxybenzyl)-2H-tetrazol-5-yl)-6-((2-(trimethyl silyl)ethyl)thio)benzenesulfonamide as a solid: LCMS (ESI) calc'd for C₃₄H₃₈BrN₅O₃S₂Si [M+H]⁺: 736, 738, found 736, 738.

### Step C: N,N-dibenzyl-3-bromo-2-(2-(4-methoxybenzyl)-2H-tetrazol-5-yl)-6-((2-(trimethyl silyl)ethyl)sulfonyl)benzenesulfonamide

To a solution of N,N-dibenzyl-3-bromo-2-(2-(4-methoxybenzyl)-2H-tetrazol-5-yl)-6-((2-(trimethylsilyl)ethyl)thio)benzenesulfonamide (33.0 g, 44.80 mmol) in DCM (300 mL) was added m-CPBA (31.0 g, 0.18 mol) slowly at room temperature. The mixture was stirred for 16 h at room temperature. The solid was filtered out. The resulting solution was quenched with the addition of aqueous saturated Na₂SO₃ (1500 mL) and extracted with of EA (3×800 mL). The organic layers were combined, washed with aqueous saturated Na₂CO₃ (3×400 mL) and brine (2×300 mL), dried over anhydrous Na₂SO₄ and filtered. The filtrate was concentrated under vacuum. The residue was purified by a silica gel column chromatography, eluted with 20% EA in PE to afford N,N-dibenzyl-3-bromo-2-(2-(4-methoxybenzyl)-2H-tetrazol-5-yl)-6-((2-(trimethylsilyl)ethyl)sulfonyl)benzenesulfonamide as a solid: LCMS (ESI) calc'd for C₃₄H₃₈BrN₅O₅S₂Si [M+H]⁺: 768, 770, found 768, 770.

### Step D: 4-Bromo-2-(N,N-dibenzylsulfamoyl)-3-(2-(4-methoxybenzyl)-2H-tetrazol-5-yl) benzenesulfinic acid

To a solution of N,N-dibenzyl-3-bromo-2-(2-(4-methoxybenzyl)-2H-tetrazol-5-yl)-6-((2-(trimethylsilyl)ethyl)sulfonyl)benzenesulfonamide (20.0 g, 26.0 mmol) in THF (200 mL) was added TBAF 3H₂O (32.8 g, 0.10 mol) at 0° C. The reaction solution was stirred at room temperature for 1 h. The resulting solution was diluted with water (600 mL) and extracted with EA (3×150 mL). The organic layers were combined, washed with aqueous saturated KHSO₄(4×300 mL) and brine (2×300 mL), dried over anhydrous MgSO₄ and filtered. The filtrate was concentrated under vacuum to afford 4-bromo-2-(N,N-dibenzylsulfamoyl)-3-(2-(4-methoxybenzyl)-2H-tetrazol-5-yl)benzenesulfinic acid as a solid which was used in the next step directly without further purification: LCMS (ESI) calc'd for C₂₉H₂₆BrN₅O₅S₂ [M+H]⁺: 668, 670, found 668, 670.

### Step E: (R)-tert-butyl (3-(4-bromo-2-(N,N-dibenzylsulfamoyl)-3-(2-(4-methoxybenzyl)-2H-tetrazol-5-yl)phenylsulfonamido)-2-hydroxypropyl)carbamate

To a solution of 4-bromo-2-(N,N-dibenzylsulfamoyl)-3-(2-(4-methoxybenzyl)-2H-tetrazol-5-yl)benzenesulfinic acid (18.1 g, 26.9 mmol) in THF (180 mL) were added (S)-tert-butyl (3-amino-2-hydroxypropyl)carbamate (7.68 g, 40.4 mmol) and TEA (5.63 mL, 40.4 mmol) at room temperature. The mixture was stirred at room temperature for 30 min. To a resulting solution was added 1-chloropyrrolidine-2,5-dione (7.19 g, 53.8 mmol). The reaction mixture was stirred at room temperature for 1 h. The resulting solution was diluted with water (500 mL) and extracted with EA (3×300 mL). The organic layers were combined, washed with brine (2×200 mL), dried over anhydrous Na₂SO₄ and filtered. The filtrate was concentrated under vacuum. The residue was purified by a silica gel column chromatography, eluted with 50% EA in PE. The fractions containing desired product were combined and concentrated under vacuum to afford (R)-tert-butyl (3-(4-bromo-2-(N,N-dibenzylsulfamoyl)-3-(2-(4-methoxybenzyl)-2H-tetrazol-5-yl)phenylsulfonamido)-2-hydroxypropyl)carbamate as a solid: LCMS (ESI) calc'd for C₃₇H₄₂BrN₇O₈S₂ [M+H]⁺: 856, 858, found 856, 858.

### Step F: (R)-tert-butyl (3-(4-bromo-2-(N,N-dibenzylsulfamoyl)-3-(2-(4-methoxybenzyl)-2H-tetrazol-5-yl)phenylsulfonamido)-2-((tert-butyldimethylsilyl)oxy)propyl)carbamate

To a solution of (R)-tert-butyl (3-(4-bromo-2-(N,N-dibenzylsulfamoyl)-3-(2-(4-methoxybenzyl)-2H-tetrazol-5-yl)phenylsulfonamido)-2-hydroxypropyl)carbamate (20.0 g, 23.34 mmol) in DMF (200 mL) were added tert-butylchlorodimethylsilane (21.0 g, 0.14 mol) and 1H-imidazole (13.0 g, 0.19 mol) at room temperature. The reaction mixture was stirred at room temperature for 16 h. The resulting solution was diluted with water (500 mL), and extracted with EA (3×250 mL). The organic layers were combined, washed with brine (2×100 mL), dried over anhydrous Na₂SO₄ and filtered. The filtrate was concentrated under vacuum. The residue was purified by a silica gel column chromatography, eluted with 30% EA in PE to afford (R)-tert-butyl (3-(4-bromo-2-(N,N-dibenzylsulfamoyl)-3-(2-(4-methoxybenzyl)-2H-tetrazol-5-yl) phenylsulfonamido)-2-((tert-butyldimethylsilyl)oxy)propyl)carbamate as a solid: LCMS (ESI) calc'd for C₄₃H₅₆BrN₇O₈S₂Si [M+H]⁺: 970, 972, found 970, 972.

### Reference Example 11

### 6,7,8,9-tetrahydropyrido[4′,3′:4,5]imidazo[1,2-a]pyrimidine

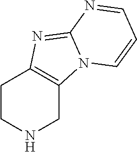 [SMILES: c1cnc2nc3c(n2c1)CNCC3]

### Step A: tert-butyl 89-dihydropyrido[4′,3′:4,5]imidazo[1,2-a]pyrimidine-7(6H)-carboxylate

Pyrimidin-2-amine (3600 mg, 37.9 mmol) and tert-butyl 3-bromo-4-oxopiperidine-1-carboxylate (5000 mg, 17.98 mmol) were heated in EtOH (18 mL) at 80° C. for 6 h. The reaction mixture was concentrated in vacuo, and purified by silica gel column chromatography twice (gradient elution 100% EtOAc to 25% MeOH/EtOAc) to give the title product. LC/MS [M+H]⁺: 275.24

### Step B: 6,7,8,9-tetrahydropyrido[4′,3′:4,5]imidazo[1,2-a]pyrimidine

tert-Butyl 8,9-dihydropyrido[4′,3′:4,5]imidazo[1,2-a]pyrimidine-7(6H)-carboxylate (2.56 g, 9.33 mmol) was stirred in TFA (5 mL) at rt for 1 h. The reaction mixture was then concentrated. The residue was eluting through an SCX ion exchange cartridge to afford the title compound as the free base LC/MS [M+H]⁺: 175.20

### Reference Example 12

### 5-(((tert-butyldimethylsilyl)oxy)methyl)-5,6,7,8-tetrahydroimidazo [1,2-a]pyrimidine

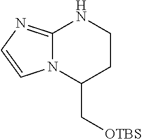 [SMILES: CC(C)(C)[Si](C)(C)OCC1CCNc2nccn21]

### Step A: 7-Oxo-5,6,7,8-tetrahydroimidazo[1,2-a]pyrimidine-5-carboxylic acid

To a stirred solution of 1H-imidazol-2-amine sulfate (2.00 g, 11.04 mmol) in water (10 mL) was added Na₂CO₃ (1.20 g, 11.04 mmol) at room temperature. The resulting solution was stirred at room temperature for 30 min. The aqueous solution was evaporated under vacuum. EtOH (20 mL) was added to the residue. The slurry was stirred for 30 min at room temperature and filtered. The filtrate was concentrated under vacuum to afford free amine. To a solution of free amine in DMF (5 mL) was added furan-2,5-dione (1.20 g, 12.14 mmol). The reaction solution was stirred at 110° C. for 2 h. The resulting solution was poured into acetone (80 mL). The solid was precipitated from the solution and filtered. The filter cake was dried under vacuum to afford the title compound, which was used in the next step without further purification: LCMS (ESI) calc'd for C₇H₇N₃O₃ [M+1]⁺: 182, found 182.

### Step B: Methyl 7-oxo-5,6,7,8-tetrahydroimidazo[1,2-a]pyrimidine-5-carboxylate

To a stirred mixture of 7-oxo-5,6,7,8-tetrahydroimidazo[1,2-a] pyrimidine-5-carboxylic acid (2.00 g, 11.04 mmol) in MeOH (10 mL) was added thionyl chloride (1.30 g, 11.04 mmol) under nitrogen at room temperature. The reaction mixture was stirred at room temperature for 16 h. The resulting mixture was concentrated under vacuum. The residue was dissolved in water (10 mL) and MeOH (5 mL) and the pH value was adjusted to 8 with aqueous saturated Na₂CO₃. Then the mixture was concentrated under vacuum and the residue was purified by silica gel column chromatography, eluting with 5% MeOH in DCM to afford the title compound: LCMS (ESI) calc'd for C₈H₉N₃O₃ [M+1]⁺: 196, found 196.

### Step C: (5,6,7,8-Tetrahydroimidazo[1,2-a]pyrimidin-5-yl)methanol

To a stirred solution of methyl 7-oxo-5,6,7,8-tetrahydroimidazo[1,2-a]pyrimidine-5-carboxylate (1.00 g, 5.12 mmol) in THF (80 mL) was added LiAlH₄ (0.58 g, 15.37 mmol) under nitrogen at 0° C. The mixture was then stirred at 50° C. for 5 h. After cooling to room temperature, the resulting mixture was quenched sequentially with water (0.1 mL), NaOH (0.1 mL, 15%) and water (0.3 mL). Then the mixture was filtered through diatomaceous earth and the filtrate was concentrated under vacuum. The residue was purified by a silica gel column chromatography, eluting with 10% MeOH in DCM to afford the title compound: LCMS (ESI) calc'd for C₇H₁₁N₃O [M+1]⁺: 154, found 154.

### Step D: 5-(((Tert-butyldimethylsilyl)oxy)methyl)-5,6,7,8-tetrahydroimidazo[1,2-a]pyrimidine

To a stirred solution of (5,6,7,8-tetrahydroimidazo[1,2-a]pyrimidin-5-yl) methanol (0.80 g, 4.18 mmol) in DMF (20 mL) were added 1H-imidazole (2.80 g, 41.80 mmol), DMAP (0.10 g, 0.84 mmol) and tert-butylchlorodimethylsilane (6.30 g, 41.80 mmol) under nitrogen at room temperature. The mixture was stirred at room temperature for 16 h. The resulting mixture was purified by reverse phase column chromatography (C₁₈) eluting with 75% MeCN in water (10 mmol/L NH₄HCO₃) to afford the title compound: LCMS (ESI) calc'd for C₁₃H₂₅N₃OSi [M+1]⁺: 268, found 268.

### Reference Examples 13 and 14

### (S)-5-(((tert-butyldimethylsilyl)oxy)methyl)-5,6,7,8-tetrahydroimidazo [1,2-a]pyrimidine and (R)-5-(((tert-butyldimethylsilyl)oxy)methyl)-5,6,7,8-tetrahydroimidazo[1,2-a]pyrimidine

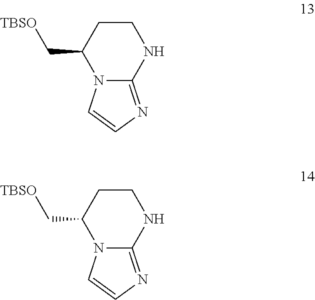 [SMILES: CC(C)(C)[Si](C)(C)OC[C@H]1CCNc2nccn21 ; CC(C)(C)[Si](C)(C)OC[C@@H]1CCNc2nccn21]

### Step A: (R)-5-(((tert-butyldimethylsilyl)oxy)methyl)-5,6,7,8-tetrahydroimidazo[1,2-a]pyrimidine and (S)-5-(((tert-butyldimethylsilyl)oxy)methyl)-5,6,7,8-tetrahydroimidazo[1,2-a]pyrimidine

0.38 g of racemic 5-(((tert-butyldimethylsilyl)oxy)methyl)-5,6,7,8-tetrahydroimidazo [1,2-a]pyrimidine was separated by Chiral-HPLC with following conditions: Column: Chiralpak IC, 2×25 cm, 5 μm; Mobile Phase A: Hex (0.1% DEA), Mobile Phase B: IPA; Flow rate: 20 mL/min; Gradient: 20 B isocratic in 21 min; Detector: UV 220/254 nm; RT1: 14.45 min; RT2: 16.77 min. The faster-eluting enantiomer was obtained 0.13 g of (R)-5-(((tert-butyldimethylsilyl)oxy)methyl)-5,6,7,8-tetrahydroimidazo [1,2-a]pyrimidine: H NMR (300 MHz, CDCl₃) δ 6.62 (d, J=1.8 Hz, 1H), 6.59 (d, J=1.8 Hz, 1H), 4.06-4.01 (m, 1H), 3.87-3.66 (m, 2H), 3.47-3.28 (m, 2H), 2.16-1.92 (m, 2H), 0.87 (s, 9H), 0.04 (s, 3H), 0.02 (s, 3H). The slower-eluting enantiomer was obtained 0.14 g of (S)-5-(((tert-butyldimethylsilyl)oxy) methyl)-5,6,7,8-tetrahydroimidazo[1,2-a]pyrimidine: ¹H NMR (300 MHz, CDCl₃): δ 6.62 (d, J=1.8 Hz, 1H), 6.59 (d, J=1.8 Hz, 1H), 4.10-3.96 (m, 1H), 3.88-3.65 (m, 2H), 3.46-3.27 (m, 2H), 2.16-1.94 (m, 2H), 0.87 (s, 9H), 0.04 (s, 3H), 0.02 (s, 3H).

### Reference Example 15

### (R)-tert-butyl (3-((2-(N,N-bis(4-methoxybenzyl)sulfamoyl)-4-iodo-3-(2-(4-methoxybenzyl)-2H-tetrazol-5-yl)phenyl)sulfonyl)-2-hydroxypropyl)carbamate

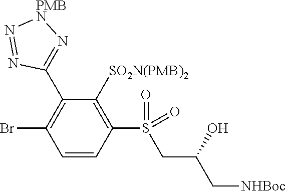

### Step A: (R)-tert-butyl(3-((2-(N,N-bis(4-methoxybenzyl)sulfamoyl)-4-iodo-3-(2-(4-methoxy benzyl)-2H-tetrazol-5-yl)phenyl)thio)-2-((tert-butyldimethylsilyl)oxy)propyl) carbamate

To a stirred solution of 6-bromo-3-iodo-N,N-bis(4-methoxybenzyl)-2-(2-(4-methoxybenzyl)-2H-tetrazol-5-yl)benzenesulfonamide (1.95 g, 2.47 mmol) (REFERENCE EXAMPLE 1), (R)-tert-butyl-2-(tert-butyldimethylsilyloxy)-3-mercaptopropylcarbamate (1.59 g, 4.94 mmol) in DMF (15 mL) was added NaH (0.20 g, 8.21 mmol) at 0° C. under nitrogen. The reaction mixture was stirred at RT for 3 hours under nitrogen. The resulting mixture was quenched with water (100 mL) and extracted with EA (3×100 mL). The combined organic layers were washed with brine (3×100 mL), dried over anhydrous Na₂SO₄ and filtered. The filtrate was concentrated under vacuum. The residue was purified by silica gel column chromatography, eluting with 50% EA in PE to afford the title compound: LCMS [M+1]⁺: 1031.

### Step B: (R)-tert-butyl(3-((2-(N,N-bis(4-methoxybenzyl)sulfamoyl)-4-iodo-3-(2-(4-methoxy benzyl)-2H-tetrazol-5-yl)phenyl)thio)-2-hydroxypropyl)carbamate

To a solution of (R)-tert-butyl-3-(2-(N,N-bis(4-methoxybenzyl)sulfamoyl)-4-iodo-3-(2-(4-methoxybenzyl)-2H-tetrazol-5-yl)phenylthio)-2-(tert-butyldimethylsilyloxy)propylcarbamate (2.2 g, 2.14 mmol) in THF (40 mL) was added TBAF (6.4 mL, 6.40 mmol) at 0° C. The reaction mixture was stirred at RT for 1 hour. The resulting mixture was quenched with water (100 mL), extracted with EA (3×100 mL). The combined organic layers were washed with sat'd aqueous KHSO₄ (5×100 mL), brine (1×100 mL), dried over anhydrous MgSO₄ and filtered. The filtrate was concentrated under vacuum. The residue was purified by silica gel column chromatography, eluting with 70% EA in PE to afford the title compound: LCMS (ESI) calc'd for C₃₉H₄₅IN₆O₈S₂ [M+1]⁺: 917, found: 917.

### Step C: (R)-tert-butyl(3-((2-(N,N-bis(4-methoxybenzyl)sulfamoyl)-4-iodo-3-(2-(4-methoxy benzyl)-2H-tetrazol-5-yl)phenyl)sulfonyl)-2-hydroxypropyl)carbamate

To a solution of (R)-tert-butyl(3-((2-(N,N-bis(4-methoxybenzyl)sulfamoyl)-4-iodo-3-(2-(4-methoxybenzyl)-2H-tetrazol-5-yl)phenyl)thio)-2-hydroxypropyl)carbamate (1.2 g, 1.31 mmol) in DCM (15 mL) was added m-CPBA (0.9 g, 5.23 mmol) at 0° C. The reaction mixture was stirred at RT for 16 hours. The resulting mixture was added into sat'd aqueous Na₂SO₃ (50 mL) and extracted with DCM (3×100 mL). The combined organic layers were washed with water (3×100 mL) and brine (3×100 mL), dried over anhydrous Na₂SO₄ and filtered. The filtrate was concentrated under vacuum. The residue was purified by silica gel column chromatography, eluting with 60% EA in PE to afford the title compound: LCMS (ESI) calc'd for C₃₉H₄₅IN₆O₁₀S₂ [M+1]⁺: 949, found: 949.

### Reference Example 16

### (R)-tert-butyl (3-((2-(N,N-bis(4-methoxybenzyl)sulfamoyl)-4-iodo-3-(2-(4-methoxybenzyl)-2H-tetrazol-5-yl)phenyl)sulfonyl)-2-((tert-butyldimethylsilyl) oxy)propyl)carbamate

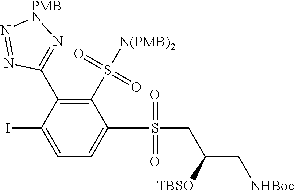

The title compound was prepared as described for REFERENCE EXAMPLE 10 step F using (R)-tert-butyl (3-((2-(N,N-bis(4-methoxybenzyl)sulfamoyl)-4-iodo-3-(2-(4-methoxybenzyl)-2H-tetrazol-5-yl)phenyl)sulfonyl)-2-hydroxypropyl)carbamate (REFERENCE EXAMPLE 15) to afford (R)-tert-butyl (3-((2-(N,N-bis(4-methoxybenzyl)sulfamoyl)-4-iodo-3-(2-(4-methoxybenzyl)-2H-tetrazol-5-yl)phenyl)sulfonyl)-2-((tert-butyldimethylsilyl)oxy)propyl)carbamate as a solid: LCMS [M+H]⁺: 1063.

### Example 1

### (R)-4-(5,6-dihydro-[1,2,4]triazolo[1,5-a]pyrazin-7(8H)-yl)-N′-(pyrrolidin-3-yl)-3-(2H-tetrazol-5-yl)benzene-1,2-disulfonamide

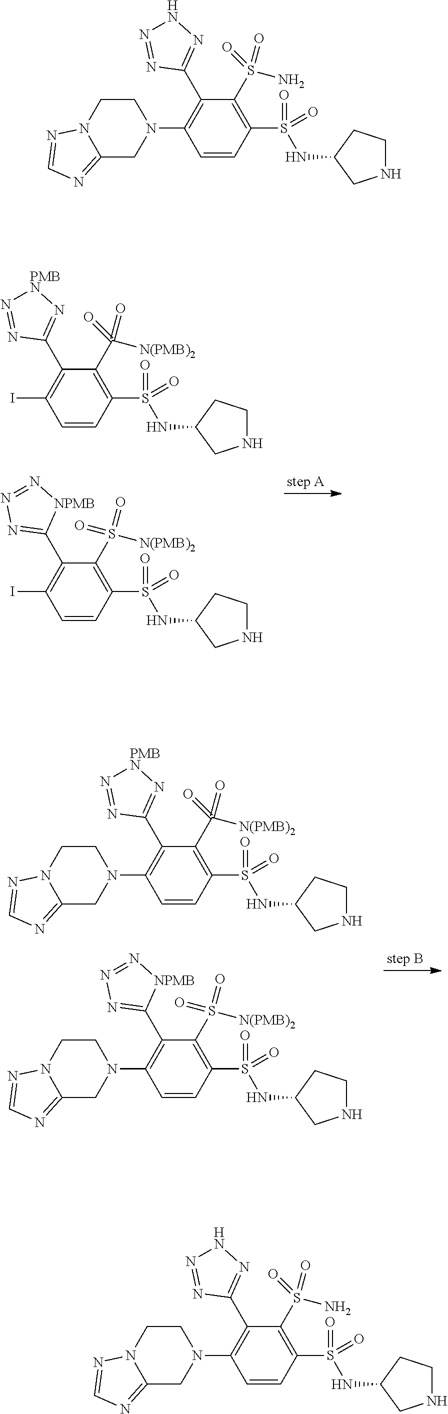

### Step A: tert-butyl (R)-3-((2-(N,N-bis(4-methoxybenzyl)sulfamoyl)-4-(5,6-dihydro-[1,2,4]triazolo[1,5-a]pyrazin-7(8H)-yl)-3-(2-(4-methoxybenzyl)-2H-tetrazol-5-yl)phenyl)sulfonamido)pyrrolidine-1-carboxylate and tert-butyl (R)-3-((2-(N,N-bis(4-methoxybenzyl)sulfamoyl)-4-(5,6-dihydro-[1,2,4]triazolo[1,5-a]pyrazin-7(8H)-yl)-3-(1-(4-methoxybenzyl)-1H-tetrazol-5-yl)phenyl)sulfonamido)pyrrolidine-1-carboxylate

A flask was charged with tert-butyl (R)-3-((2-(N,N-bis(4-methoxybenzyl)sulfamoyl)-4-iodo-3-(1-(4-methoxybenzyl)-1H-tetrazol-5-yl)phenyl)sulfonamido)pyrrolidine-1-carboxylate and tert-butyl (R)-3-((2-(N,N-bis(4-methoxybenzyl)sulfamoyl)-4-iodo-3-(2-(4-methoxybenzyl)-2H-tetrazol-5-yl)phenyl)sulfonamido)pyrrolidine-1-carboxylate (REFERENCE EXAMPLE 5) (0.15 g, 0.156 mmol), 5,6,7,8-tetrahydro-[1,2,4]triazolo[1,5-a]pyrazine (0.039 g, 0.313 mmol), Cs₂CO₃ (0.153 g, 0.469 mmol) and rac-BINAP-Pd-G3 precatalyst (0.031 g, 0.031 mmol). The vial was sealed, degassed with N₂, and filled with DME (1.6 mL). The resulting mixture was heated overnight at 80° C. The reaction mixture was filtered through diatomaceous earth. The filtrate was concentrated and the residue was purified by silica gel column chromatography using (0-10%) MeOH/DCM as the mobile phase to afford the title compound. LC/MS [M+H]⁺: 956.81.

### Step B: (R)-4-(5,6-dihydro-[1,2,4]triazolo[1,5-a]pyrazin-7(8H)-yl)-N′-(pyrrolidin-3-yl)-3-(2H-tetrazol-5-yl)benzene-1,2-disulfonamide

To the solution of tert-butyl (R)-3-((2-(N,N-bis(4-methoxybenzyl)sulfamoyl)-4-(5,6-dihydro-[1,2,4]triazolo[1,5-a]pyrazin-7(8H)-yl)-3-(2-(4-methoxybenzyl)-2H-tetrazol-5-yl)phenyl)sulfonamido)pyrrolidine-1-carboxylate and tert-butyl (R)-3-((2-(N,N-bis(4-methoxybenzyl)sulfamoyl)-4-(5,6-dihydro-[1,2,4]triazolo[1,5-a]pyrazin-7(8H)-yl)-3-(1-(4-methoxybenzyl)-1H-tetrazol-5-yl)phenyl)sulfonamido)pyrrolidine-1-carboxylate (70 mg, 0.073 mmol) in DCM (2.5 mL) was added anisole (0.080 mL, 0.732 mmol) and TFA (0.56 mL, 7.32 mmol) at 0° C. The reaction mixture was stirred at 0° C. for 30 min. After removing the volatiles under vacuum, the residue was dissolved in TFA (2.24 mL, 29.3 mmol). The resulting mixture was stirred at 80° C. for 1.0 h. After removing the volatiles under vacuum the residue was purified by reverse phase HPLC (gradient 3-30% MeCN/water as eluent, 0.1% NH₄OH as additive) to give title compound. LC/MS [M+H]⁺: 496.50.

### Example 2

### (R)-4-(5,6-dihydroimidazo[1,5-a]pyrazin-7(8H)-yl)-N¹-(pyrrolidin-3-yl)-3-(2H-tetrazol-5-yl)benzene-1,2-disulfonamide

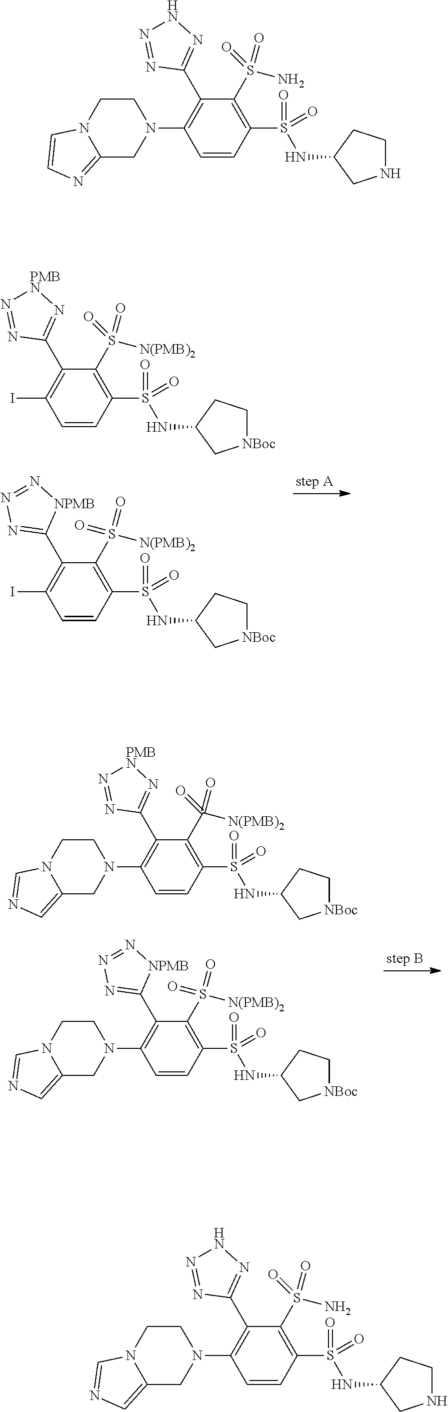

### Step A: tert-butyl (R)-3-((2-(N,N-bis(4-methoxybenzyl)sulfamoyl)-4-(5,6-dihydroimidazo[1,5-a]pyrazin-7(8H)-yl)-3-(2-(4-methoxybenzyl)-2H-tetrazol-5-yl)phenyl)sulfonamido)pyrrolidine-1-carboxylate and tert-butyl (R)-3-((2-(N,N-bis(4-methoxybenzyl)sulfamoyl)-4-(5,6-dihydroimidazo[1,5-a]pyrazin-7(8H)-yl)-3-(1-(4-methoxybenzyl)-1H-tetrazol-5-yl)phenyl)sulfonamido)pyrrolidine-1-carboxylate

A flask was charged with tert-butyl (R)-3-((2-(N,N-bis(4-methoxybenzyl)sulfamoyl)-4-iodo-3-(1-(4-methoxybenzyl)-1H-tetrazol-5-yl)phenyl)sulfonamido)pyrrolidine-1-carboxylate and tert-butyl (R)-3-((2-(N,N-bis(4-methoxybenzyl)sulfamoyl)-4-iodo-3-(2-(4-methoxybenzyl)-2H-tetrazol-5-yl)phenyl)sulfonamido)pyrrolidine-1-carboxylate (REFERENCE EXAMPLE 5) (0.15 g, 0.156 mmol), 5,6,7,8-tetrahydroimidazo[1,5-a]pyrazine (0.038 g, 0.313 mmol), Cs₂CO₃ (0.153 g, 0.469 mmol) and Pd(dppf)Cl₂ (0.023 g, 0.031 mmol). The vial was sealed, degassed with N₂, and filled with DME (1.56 mL). The resulting mixture was heated overnight at 80° C. The reaction mixture was filtered through diatomaceous earth. The filtrate was concentrated and the residue purified by silica gel column chromatography using (gradient 0-10%) MeOH in DCM as the mobile phase to afford the title compound. LC/MS [M+H]⁺: 955.82.

### Step B: (R)-4-(5,6-dihydroimidazo[1,5-a]pyrazin-7(8H)-yl)-N′-(pyrrolidin-3-yl)-3-(2H-tetrazol-5-yl)benzene-1,2-disulfonamide

Step B was conducted in a similar fashion to Step B of Example 1 to afford the title compound. LC/MS [M+H]⁺: 495.00.

### Example 3

### (R)—N¹-(3-amino-2-hydroxypropyl)-4-(2-amino-3,4,6,7-tetrahydro-5H-imidazo[4,5-c]pyridin-5-yl)-3-(2H-tetrazol-5-yl)benzene-1,2-disulfonamide

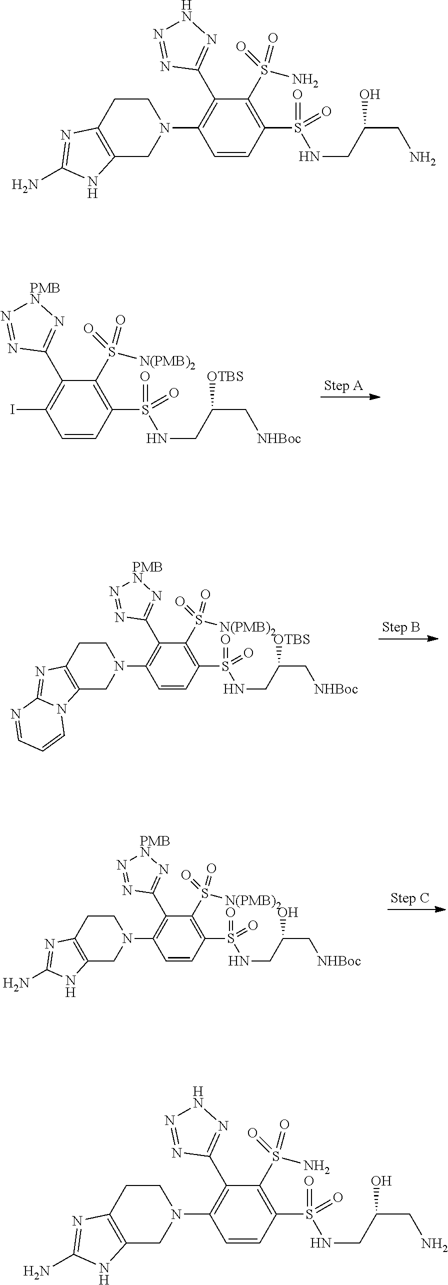

### Step A: tert-butyl (R)-(3-((2-(N,N-bis(4-methoxybenzyl)sulfamoyl)-4-(8,9-dihydropyrido[4′,3′:4,5]imidazo[1,2-a]pyrimidin-7(6H)-yl)-3-(2-(4-methoxybenzyl)-2H-tetrazol-5-yl)phenyl)sulfonamido)-2-((tert-butyldimethylsilyl)oxy)propyl)carbamate

6,7,8,9-Tetrahydropyrido[4′,3′:4,5]imidazo[1,2-a]pyrimidine (194 mg, 1.113 mmol) (REFERENCE EXAMPLE 11), tert-Butyl (R)-(3-((2-(N,N-bis(4-methoxybenzyl)sulfamoyl)-4-iodo-3-(2-(4-methoxybenzyl)-2H-tetrazol-5-yl)phenyl)sulfonamido)-2-((tert-butyldimethylsilyl)oxy)propyl)carbamate (REFERENCE EXAMPLE 7) (600 mg, 0.557 mmol), Cs₂CO₃ (544 mg, 1.670 mmol), rac-BINAP-G3 precatalyst (110 mg, 0.111 mmol) were placed in a vial. After addition of DME (5.6 mL), the reaction mixture was degassed with N₂ and heated at 80° C. overnight. After concentration, the residue was purified by silica gel column chromatography (100% hexane to 80 then to 100% EtOAc/EtOH (3/1) over hexane) to give the title product. [M+H]⁺: 1124.77.

### Step B: tert-butyl (R)-(3-((4-(2-amino-3,4,6,7-tetrahydro-5H-imidazo[4,5-c]pyridin-5-yl)-2-(N,N-bis(4-methoxybenzyl)sulfamoyl)-3-(2-(4-methoxybenzyl)-2H-tetrazol-5-yl)phenyl)sulfonamido)-2-((tert-butyldimethylsilyl)oxy)propyl)carbamate

tert-Butyl ((2R)-3-(2-(N,N-bis(4-methoxybenzyl)sulfamoyl)-4-(8,9-dihydropyrido[4′,3′:4,5]imidazo[1,2-a]pyrimidin-7(6H)-yl)-3-(1-(4-methoxybenzyl)-1H-tetrazol-5-yl)phenylsulfonamido)-2-((tert-butyldimethylsilyl)oxy)propyl)carbamate (194 mg, 0.173 mmol) was dissolved in EtOH (1 mL), followed by addition of hydrazine (54.2 μL, 1.725 mmol) and water (15 uL). The reaction mixture was heated at 80° C. for 1 h. After concentration, the residue was purified by silica gel column chromatography (100% hexane to 100% EtOAc/EtOH (3/1)) to give the title product. LC/MS [M+H]⁺: 1088.86.

### Step C: (R)—N¹-(3-amino-2-hydroxypropyl)-4-(2-amino-3,4,6,7-tetrahydro-5H-imidazo[4,5-c]pyridin-5-yl)-3-(2H-tetrazol-5-yl)benzene-1,2-disulfonamide

Tert-Butyl ((2R)-3-(4-(2-amino-6,7-dihydro-3H-imidazo[4,5-c]pyridin-5(4H)-yl)-2-(N,N-bis(4-methoxybenzyl)sulfamoyl)-3-(1-(4-methoxybenzyl)-1H-tetrazol-5-yl)phenylsulfonamido)-2-((tert-butyldimethylsilyl)oxy)propyl)carbamate (72 mg, 0.066 mmol) was dissolved in DCM (1.5 mL) and treated with TFA (1 mL). The mixture was stirred at RT for 1 h. The reaction mixture was then concentrated and co-evaporated with toluene. The residue was heated in TFA (2 mL) at 80° C. for 50 min. After addition of water (0.2 mL), the reaction mixture was stirred with continued heating at 80° C. for 30 min. After concentration, the residue was purified with reversed phase HPLC (0-40% CH₃CN/water with 0.1% NH₄OH) and followed by an additional purification with reversed phase HPLC (0-40% CH₃CN/water with 0.1% TFA). The fractions were lyophilized to give the title compound. LC/MS [M+H]⁺: 514.30.

### Example 4

### (R)—N¹-(pyrrolidin-3-yl)-3-(2H-tetrazol-5-yl)-4-(2-(trifluoromethyl)-5,6-dihydro-[1,2,4]triazolo[1,5-a]pyrazin-7(8H)-yl)benzene-1,2-disulfonamide

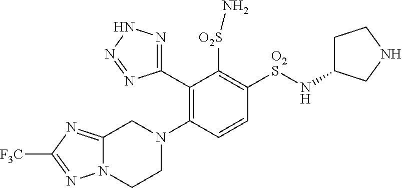 [SMILES: NS(=O)(=O)c1c(S(=O)(=O)N[C@@H]2CCNC2)ccc(N2CCn3nc(C(F)(F)F)nc3C2)c1-c1nn[nH]n1]

### Step A: tert-butyl (R)-3-((2-(N,N-dibenzylsulfamoyl)-3-(2-(4-methoxybenzyl)-2H-tetrazol-5-yl)-4-(2-(trifluoromethyl)-5,6-dihydro-[1,2,4]triazolo[1,5-a]pyrazin-7(8H)-yl)phenyl)sulfonamido)pyrrolidine-1-carboxylate

A suspension of Rac-BINAP-Pd-G3 precatalyst (46.5 mg, 0.047 mmol), 2-(trifluoromethyl)-5,6,7,8-tetrahydro-[1,2,4]triazolo[1,5-a]pyrazine (90 mg, 0.469 mmol), (R)-tert-butyl 3-(4-bromo-2-(N,N-dibenzylsulfamoyl)-3-(2-(4-methoxybenzyl)-2H-tetrazol-5-yl)phenylsulfonamido)pyrrolidine-1-carboxylate (REFERENCE EXAMPLE 9) (200 mg, 0.235 mmol) and Cs₂CO₃ (229 mg, 0.704 mmol) in DME (4 mL) was degassed for 20 min. The reaction mixture was heated to 80° C. for over night. The reaction mixture was cooled down to rt, then was chromatographed over silica gel (0-10% MeOH in DCM as eluent) to give the title product. LCMS [M+H]⁺ 964.2.

### Step B: (R)—N¹-(pyrrolidin-3-yl)-3-(2H-tetrazol-5-yl)-4-(2-(trifluoromethyl)-5,6-dihydro-[1,2,4]triazolo[1,5-a]pyrazin-7(8H)-yl)benzene-1,2-disulfonamide

To a solution of (R)-tert-butyl 3-(2-(N,N-dibenzylsulfamoyl)-3-(2-(4-methoxybenzyl)-2H-tetrazol-5-yl)-4-(2-(trifluoromethyl)-5,6-dihydro-[1,2,4]triazolo[1,5-a]pyrazin-7(81)-yl)phenylsulfonamido)pyrrolidine-1-carboxylate (120 mg, 0.124 mmol) in DCM (0.5 mL) was added anisole (0.13 mL, 1.24 mmol) and TFA (0.5 mL, 6.2 mmol) at 0′C. The reaction was stirred at 0° C. for 30 min. After that, the reaction mixture was concentrated. The residue was dissolved in 2 mL of methanol, and then was filtered through anion-exchange cartridge (Agilent, BE-SCX 2 g) with 20 mL of methanol (containing 7N ammonia) as eluent. The filtrate was collected and concentrated. The residue was dissolved in sulfuric acid (0.5 mL, 9.33 mmol) at 0′C. The reaction mixture was stirred at RT overnight. To the reaction mixture was added 20 mL of ammonia in methanol (7.0N) at 0° C. to provide a suspension. The mixture was filtered and the filtrate was concentrated. The residue was dissolved in 2 mL of DMSO, then was purified by reverse phase HPLC (0-30% acetonitrile in water) to give the title compound. LC-MS [M+H]⁺ 564.0.

EXAMPLES 5-7 in the Table below were prepared in an analogous fashion to that described for EXAMPLE 4 starting from the requisite amines and (R)-tert-butyl 3-(4-bromo-2-(N,N-dibenzylsulfamoyl)-3-(2-(4-methoxybenzyl)-2H-tetrazol-5-yl)phenylsulfonamido)pyrrolidine-1-carboxylate (REFERENCE EXAMPLE 9).

| | | | | Calc'd | |
|---|---|---|---|---|---|
| Ex. | | | | Mass | LC/MS |
| No. | RR′NH | Structure | Name | [M + H]⁺ | [M + H]⁺ |
| 5 | 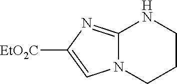 [SMILES: CCOC(=O)c1cn2c(n1)NCCC2] | 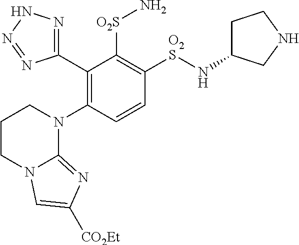 [SMILES: CCOC(=O)c1cn2c(n1)N(c1ccc(S(=O)(=O)N[C@@H]3CCNC3)c(S(N)(=O)=O)c1-c1nn[nH]n1)CCC2] | ethyl (R)-8-(4-(N- (pyrrolidin-3- yl)sulfamoyl)-3- sulfamoyl-2-(2H- tetrazol-5- yl)phenyl)-5,6,7,8- tetrahydroimidazo [1,2-a]pyrimidine- 2-carboxylate | 566.6 | 566.7 |
| 6 | 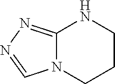 | 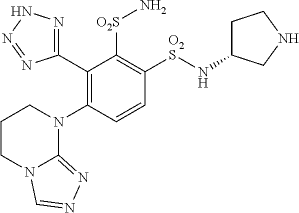 [SMILES: NS(=O)(=O)c1c(S(=O)(=O)N[C@@H]2CCNC2)ccc(N2CCCn3cnnc32)c1-c1nn[nH]n1] | (R)-4-(6,7- dihydro- [1,2,4]triazolo [4,3-a]pyrimidin- 8(5H)-yl)-N¹- (pyrrolidin-3-yl)- 3-(2H-tetrazol-5- yl)benzene-1,2- disulfonamide | 495.5 | 495.5 |
| 7 | 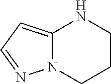 | 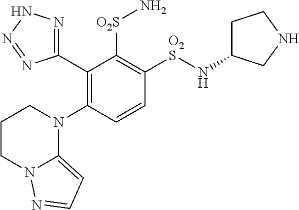 [SMILES: NS(=O)(=O)c1c(S(=O)(=O)N[C@@H]2CCNC2)ccc(N2CCCn3nccc32)c1-c1nn[nH]n1] | (R)-4-(6,7- dihydropyrazolo [1,5-a]pyrimidin- 4(5H)-yl)-N¹- (pyrrolidin-3-yl)- 3-(2H-tetrazol-5- yl)benzene-1,2- disulfonamide | 495.5 | 495.2 |

### Example 8

### (R)-4-(6,7-dihydro-[1,2,4]triazolo[1,5-a]pyrimidin-4(5H)-yl)-N1-(pyrrolidin-3-yl)-3-(1H-tetrazol-5-yl)benzene-1,2-disulfonamide

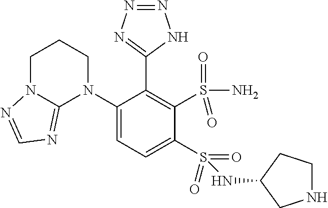 [SMILES: NS(=O)(=O)c1c(S(=O)(=O)N[C@@H]2CCNC2)ccc(N2CCCn3ncnc32)c1-c1nnn[nH]1]

### Step A: tert-butyl (R)-3-((2-(N,N-bis(4-methoxybenzyl)sulfamoyl)-4-iodo-3-(2-(4-methoxybenzyl)-2H-tetrazol-5-yl)phenyl)sulfonamido)pyrrolidine-1-carboxylate

(R)-tert-butyl 3-aminopyrrolidine-1-carboxylate (1.13 mL, 6.10 mmol), triethylamine (1.28 mL, 9.15 mmol) were added to a stirred mixture of 2-(N,N-bis(4-methoxybenzyl)sulfamoyl)-4-iodo-3-(2-(4-methoxybenzyl)-2H-tetrazol-5-yl)benzenesulfinic acid (REFERENCE EXAMPLE 3) (2.37 g, 3.05 mmol) in dichloromethane (40 mL) at 0° C. 1-chloropyrrolidine-2,5-dione (0.896 g, 6.71 mmol) was added to the reaction mixture and the mixture was stirred at 0° C. for 45 min. The mixture was diluted with water (80 mL), extracted with ethyl acetate (2×100 mL). The residue was purified by column chromatography on silica gel, eluting with 0-100% EtOAc/isohexane to give the title compound. LC/MS [M+H]+: 960.

### Step B: tert-butyl (R)-3-((2-(N,N-bis(4-methoxybenzyl)sulfamoyl)-4-(6,7-dihydro-[1,2,4]triazolo[1,5-a]pyrimidin-4(5H)-yl)-3-(1-(4-methoxybenzyl)-1H-tetrazol-5-yl)phenyl)sulfonamido)pyrrolidine-1-carboxylate

To (3R)-tert-butyl 3-(2-(N,N-bis(4-methoxybenzyl)sulfamoyl)-4-iodo-3-(1-(4-methoxybenzyl)-1H-tetrazol-5-yl)phenylsulfonamido)pyrrolidine-1-carboxylate (200 mg, 0.208 mmol) and 4,5,6,7-tetrahydro-[1,2,4]triazolo[1,5-a]pyrimidine (51.7 mg, 0.417 mmol), Cs₂CO₃ (204 mg, 0.625 mmol), and Pd(dppf)Cl₂ (40 mg) were added DME (3 mL) at RT and the mixture was degassed with N₂ for 10 min, and then stirred at 80° C. overnight. The mixture was diluted with water (50 mL), extracted with CH₂Cl₂ (2×50 mL). The combined organic phases were washed with brine, dried (MgSO₄) and concentrated under reduced pressure. The residue was purified by column chromatography on silica gel, eluting with 0-60% EtOAc/Ethanol(3:1)/Heptane in to give the title product. LC/MS [M+H]+: 956.

### Step C: (R)-4-(6,7-dihydro-[1,2,4]triazolo[1,5-a]pyrimidin-4(5H)-yl)-N2-(4-methoxybenzyl)-3-(1-(4-methoxybenzyl)-1H-tetrazol-5-yl)-N1-(pyrrolidin-3-yl)benzene-1,2-disulfonamide

1,4-Dimethoxybenzene (195 mg, 1.412 mmol) and TFA (2 mL, 26.0 mmol) were added to a stirred solution of starting material (3R)-tert-butyl 3-(2-(N,N-bis(4-methoxybenzyl)sulfamoyl)-4-(6,7-dihydro-[1,2,4]triazolo[1,5-a]pyrimidin-4(5H)-yl)-3-(1-(4-methoxybenzyl)-1H-tetrazol-5-yl)phenylsulfonamido)pyrrolidine-1-carboxylate (90 mg, 0.094 mmol) in dichloromethane (2 mL) at RT and the mixture was stirred at RT for 2 h. The mixture was concentrated to provide the product that was used without further purification. LC/MS [M+H]+: 736.

### Step D: (R)-4-(6,7-dihydro-[1,2,4]triazolo[1,5-a]pyrimidin-4(5H)-yl)-N1-(pyrrolidin-3-yl)-3-(1H-tetrazol-5-yl)benzene-1,2-disulfonamide

1,4-Dimethoxybenzene (130 mg, 0.942 mmol) and TFA (2 mL, 60 mmol) were added to a stirred solution of 4-(6,7-dihydro-[1,2,4]triazolo[1,5-a]pyrimidin-4(5H)-yl)-N2-(4-methoxybenzyl)-3-(1-(4-methoxybenzyl)-1H-tetrazol-5-yl)-N1-((R)-pyrrolidin-3-yl)benzene-1,2-disulfonamide (69.3 mg, 0.094 mmol) at RT and the mixture was stirred at 80° C. for 90 min. The mixture was concentrated. The residue was purified by preparative HPLC reverse phase (C18), eluting with acetonitrile/water with 0.05% NH₃ to give the title compound LC/MS [M+H]⁺: 496.

### Example 9

### (R)-4-(5,6-dihydroimidazo[1,2-a]pyrazin-7(8H)-yl)-N1-(pyrrolidin-3-yl)-3-(1H-tetrazol-5-yl)benzene-12-disulfonamide

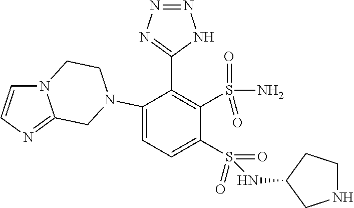 [SMILES: NS(=O)(=O)c1c(S(=O)(=O)N[C@@H]2CCNC2)ccc(N2CCn3ccnc3C2)c1-c1nnn[nH]1]

### Step A: tert-butyl (R)-3-((2-(N,N-bis(4-methoxybenzyl)sulfamoyl)-4-(5,6-dihydroimidazo[1,2-a]pyrazin-7(8H)-yl)-3-(1-(4-methoxybenzyl)-1H-tetrazol-5-yl)phenyl)sulfonamido)pyrrolidine-1-carboxylate

To (3R)-tert-butyl 3-(2-(N,N-bis(4-methoxybenzyl)sulfamoyl)-4-iodo-3-(1-(4-methoxybenzyl)-1H-tetrazol-5-yl)phenylsulfonamido)pyrrolidine-1-carboxylate (REFERENCE EXAMPLE 5) (120 mg, 0.125 mmol) and 5,6,7,8-tetrahydroimidazo[1,2-a]pyrazine (30.8 mg, 0.250 mmol), Cs₂CO₃ (122 mg, 0.375 mmol), and RAC-BINAP-Pd-G3 precatalyst (25 mg, 0.025 mmol) were added to DME (2.5 mL) at RT and the mixture was degassed for 10 min under nitrogen, stirred at 80° C. overnight. The mixture was diluted with water (50 mL), extracted with EtOAc (2×50 mL). The combined organic phases were washed with brine, dried (MgSO₄) and concentrated under reduced pressure. The residue was purified by column chromatography on silica gel, eluting with a gradient of 0-25% heptane/ethanol over 20 min to give the title product. LC/MS [M+H]: 955.

### Step B: (R)-4-(5,6-dihydroimidazo[1,2-a]pyrazin-7(8H)-yl)-N2-(4-methoxybenzyl)-3-(1-(4-methoxybenzyl)-1H-tetrazol-5-yl)-N1-(pyrrolidin-3-yl)benzene-1,2-disulfonamide

1,4-Dimethoxybenzene (57.9 mg, 0.419 mmol) and TFA (1.5 mL, 19.5 mmol) were added to a stirred solution of starting material (3R)-tert-butyl 3-(2-(N,N-bis(4-methoxybenzyl)sulfamoyl)-4-(5,6-dihydroimidazo[1,2-a]pyrazin-7(8H)-yl)-3-(1-(4-methoxybenzyl)-1H-tetrazol-5-yl)phenylsulfonamido)pyrrolidine-1-carboxylate (40 mg, 0.042 mmol) in dichloromethane (2 mL) at RT and the mixture was stirred at RT for 90 min. The mixture was concentrated. The residue was used without further purification. LC/MS+ [M+H]: 735.

### Step C: Synthesis of (R)-4-(5,6-dihydroimidazo[1,2-a]pyrazin-7(8H)-yl)-N1-(pyrrolidin-3-yl)-3-(1H-tetrazol-5-yl)benzene-1,2-disulfonamide

1,4-Dimethoxybenzene (57.9 mg, 0.419 mmol) and TFA (2 mL, 19.47 mmol) were added to a stirred solution of starting material 4-(5,6-dihydroimidazo[1,2-a]pyrazin-7(8H)-yl)-N2-(4-methoxybenzyl)-3-(1-(4-methoxybenzyl)-1H-tetrazol-5-yl)-N1-((R)-pyrrolidin-3-yl)benzene-1,2-disulfonamide (30.8 mg, 0.042 mmol) at RT and the mixture was stirred at 80° C. for 90 min. The mixture was concentrated. The residue was purified by preparative HPLC reverse phase (C18), eluting with 0-25% acetonitrile/water with 0.05% NH₃ to give the title compound after lyophilization. LC/MS [M+H]+: 496.

### Example 10

### N1-(1,3-diaminopropan-2-yl)-4-(5,6-dihydroimidazo[1,2-a]pyrazin-7(8H)-yl)-3-(2H-tetrazol-5-yl)benzene-1,2-disulfonamide

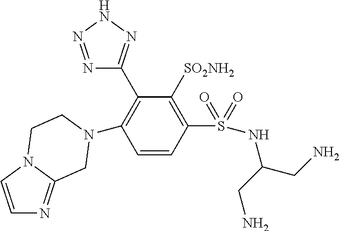 [SMILES: NCC(CN)NS(=O)(=O)c1ccc(N2CCn3ccnc3C2)c(-c2nn[nH]n2)c1S(N)(=O)=O]

### Step A: di-tert-butyl (2-((2-(N,N-bis(4-methoxybenzyl)sulfamoyl)-4-iodo-3-(2-(4-methoxybenzyl)-2H-tetrazol-5-yl)phenyl)sulfonamido)propane-1,3-diyl)dicarbamate

Triethylamine (0.761 mL, 5.41 mmol), DMAP (0.221 g, 1.805 mmol) and di-tert-butyl (2-aminopropane-1,3-diyl)dicarbamate (1.045 g, 3.61 mmol) were added to a stirred solution of starting material 2-(N,N-bis(4-methoxybenzyl)sulfamoyl)-4-iodo-3-(2-(4-methoxybenzyl)-2H-tetrazol-5-yl)benzenesulfinic acid (REFERENCE EXAMPLE 3) (1.4 g, 1.805 mmol) in dichloromethane (15 mL) at 0° C. and 1-chloropyrrolidine-2,5-dione (0.530 g, 3.97 mmol) was added. The mixture was stirred at 0° C. for 45 min. The mixture was diluted with water (50 mL), extracted with dichloromethane (2×50 mL). The combined organic phases were washed with brine, dried (MgSO₄) and concentrated under reduced pressure. The residue was purified by column chromatography on silica gel, eluting with 0-30% Heptane/Ethanol in 30 min to give the title compound. LC/MS [M+H]⁺: 1064.

### Steps B and C

Di-tert-butyl (2-((2-(N,N-bis(4-methoxybenzyl)sulfamoyl)-4-iodo-3-(2-(4-methoxybenzyl)-2H-tetrazol-5-yl)phenyl)sulfonamido)propane-1,3-diyl)dicarbamate was converted to EXAMPLE 10 using procedures analogous to those described in Steps A, B and C of EXAMPLE 9. LC/MS [M+H]+: 498.

### Example 11

### N1-(1,3-diaminopropan-2-yl)-4-(6,7-dihydroimidazo[1,2-a]pyrimidin-8(5H)-yl)-3-(2H-tetrazol-5-yl)benzene-1,2-disulfonamide

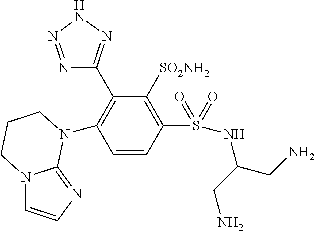 [SMILES: NCC(CN)NS(=O)(=O)c1ccc(N2CCCn3ccnc32)c(-c2nn[nH]n2)c1S(N)(=O)=O]

### Step A: di-tert-butyl (2-((2-(N,N-bis(4-methoxybenzyl)sulfamoyl)-4-(6,7-dihydroimidazo[1,2-a]pyrimidin-8(5H)-yl)-3-(2-(4-methoxybenzyl)-2H-tetrazol-5-yl)phenyl)sulfonamido)propane-1,3-diyl)dicarbamate

To di-tert-butyl (2-(2-(N,N-bis(4-methoxybenzyl)sulfamoyl)-4-iodo-3-(2-(4-methoxybenzyl)-2H-tetrazol-5-yl)phenylsulfonamido)propane-1,3-diyl)dicarbamate (from EXAMPLE 10 Step A) (250 mg, 0.235 mmol) and 5,6,7,8-tetrahydroimidazo[1,2-a]pyrimidine (57.9 mg, 0.470 mmol), Cs₂CO₃ (230 mg, 0.706 mmol), and RAC-BINAP-PD-G3 precatalyst (46.7 mg, 0.047 mmol) were added DME (3 mL) at RT and the mixture was degassed for 10 min. The reaction mixture was then stirred at 80° C. overnight. The mixture was diluted with water (50 mL), extracted with CH₂Cl₂ (2×50 mL). The combined organic phases were washed with brine, dried (MgSO₄) and concentrated under reduced pressure. The residue was purified by column chromatography on silica gel, eluting with 0-30% heptane/ethanol in 30 min to give title product. LC/MS [M+H]+: 1058.

### Step B: N1-(1,3-diaminopropan-2-yl)-4-(6,7-dihydroimidazo[1,2-a]pyrimidin-8(5H)-yl)-N2-(4-methoxybenzyl)-3-(2-(4-methoxybenzyl)-2H-tetrazol-5-yl)benzene-1,2-disulfonamide

TFA (2 mL, 26.0 mmol) and 1,4-dimethoxybenzene (166 mg, 1.205 mmol) were added to a stirred solution of starting material di-tert-butyl (2-(2-(N,N-bis(4-methoxybenzyl)sulfamoyl)-4-(6,7-dihydroimidazo[1,2-a]pyrimidin-8(5H)-yl)-3-(2-(4-methoxybenzyl)-2H-tetrazol-5-yl)phenylsulfonamido)propane-1,3-diyl)dicarbamate (85 mg, 0.080 mmol) in dichloromethane (2 mL) at RT and the mixture was stirred at RT for 90 min. The mixture was concentrated to obtain the crude product and used without further purification. LC/MS [M+H]+: 738.

### Step C: N1-(1,3-diaminopropan-2-yl)-4-(6,7-dihydroimidazo[1,2-a]pyrimidin-8(5H)-yl)-3-(2H-tetrazol-5-yl)benzene-1,2-disulfonamide

1,4-Dimethoxybenzene (111 mg, 0.804 mmol) and TFA (2 mL, 26.0 mmol) were added to a stirred solution of starting material N1-(1,3-diaminopropan-2-yl)-4-(6,7-dihydroimidazo[1,2-a]pyrimidin-8(5H)-yl)-N2-(4-methoxybenzyl)-3-(2-(4-methoxybenzyl)-2H-tetrazol-5-yl)benzene-1,2-disulfonamide (59.3 mg, 0.080 mmol) in TFA at RT and the mixture was stirred at 80° C. for 90 min. The mixture was concentrated. The residue was purified by preparative HPLC reverse phase (C₁₈), gradient elution 0-30% Acetonitrile/Water with 0.05% NH₃ to give the title compound after lyophilization. LC/MS [M+H]+: 498.

EXAMPLES 12-18 in the Table below were prepared in an analogous fashion to that described in EXAMPLE 10 Step A and EXAMPLE 11 using the requisite amine as shown in the table.

| | | | | | LC/MS |
|---|---|---|---|---|---|
| Ex. | Structure | Name | Amine | MW | [M + H]⁺ |
| 12 | 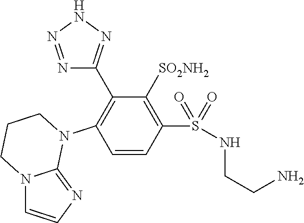 [SMILES: NCCNS(=O)(=O)c1ccc(N2CCCn3ccnc32)c(-c2nn[nH]n2)c1S(N)(=O)=O] | N1-(2- aminoethyl)-4- (6,7- dihydroimidazo[1, 2-a]pyrimidin- 8(5H)-yl)-3-(2H- tetrazol-5- yl)benzene-1,2- disulfonamide | 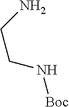 [SMILES: CC(C)(C)OC(=O)NCCN] | 468 | 469 |
| 13 | 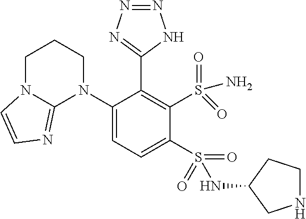 [SMILES: NS(=O)(=O)c1c(S(=O)(=O)N[C@@H]2CCNC2)ccc(N2CCCn3ccnc32)c1-c1nnn[nH]1] | (R)-4-(6,7- dihydroimidazo[1, 2-a]pyrimidin- 8(5H)-yl)-N1- (pyrrolidin-3-yl)- 3-(1H-tetrazol-5- yl)benzene-1,2- disulfonamide | 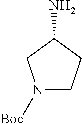 [SMILES: CC(C)(C)OC(=O)N1CC[C@@H](N)C1] | 494 | 495 |
| 14 | 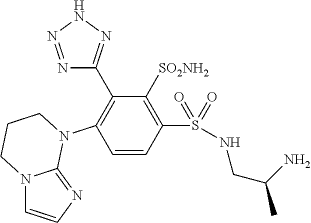 [SMILES: C[C@H](N)CNS(=O)(=O)c1ccc(N2CCCn3ccnc32)c(-c2nn[nH]n2)c1S(N)(=O)=O] | (S)-N1-(2- aminopropyl)-4- (6,7- dihydroimidazo[1, 2-a]pyrimidin- 8(5H)-yl)-3-(2H- tetrazol-5- yl)benzene-1,2- disulfonamide | 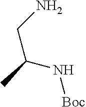 | 482 | 483 |
| 15 | 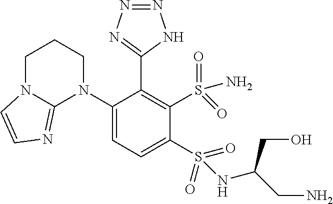 [SMILES: NC[C@H](CO)NS(=O)(=O)c1ccc(N2CCCn3ccnc32)c(-c2nnn[nH]2)c1S(N)(=O)=O] | (R)-N1-(1-amino- 3-hydroxypropan- 2-yl)-4-(6,7- dihydroimidazo[1, 2-a]pyrimidin- 8(5H)-yl)-3-(1H- tetrazol-5- yl)benzene-1,2- disulfonamide | 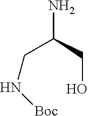 [SMILES: CC(C)(C)OC(=O)NC[C@@H](N)CO] | 498 | 499 |
| 16 | 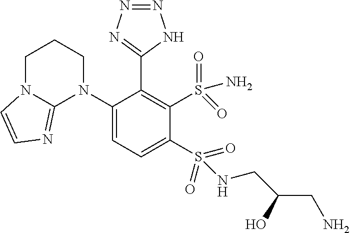 [SMILES: NC[C@@H](O)CNS(=O)(=O)c1ccc(N2CCCn3ccnc32)c(-c2nnn[nH]2)c1S(N)(=O)=O] | (R)-N1-(3-amino- 2-hydroxypropyl)- 4-(6,7- dihydroimidazo[1, 2-a]pyrimidin- 8(5H)-yl)-3-(1H- tetrazol-5- yl)benzene-1,2- disulfonamide | 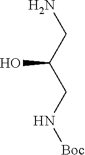 [SMILES: CC(C)(C)OC(=O)NC[C@@H](O)CN] | 498 | 499 |
| 17 | 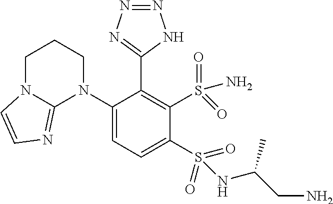 [SMILES: C[C@H](CN)NS(=O)(=O)c1ccc(N2CCCn3ccnc32)c(-c2nnn[nH]2)c1S(N)(=O)=O] | (R)-N1-(1- aminopropan-2- yl)-4-(6,7- dihydroimidazo[1, 2-a]pyrimidin- 8(5H)-yl)-3-(1H- tetrazol-5- yl)benzene-1,2- disulfonamide | 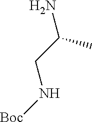 | 482 | 483 |
| 18 | 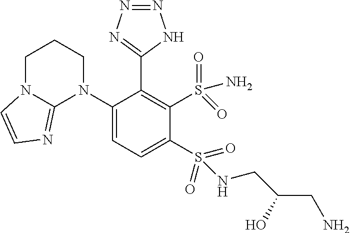 [SMILES: NC[C@H](O)CNS(=O)(=O)c1ccc(N2CCCn3ccnc32)c(-c2nnn[nH]2)c1S(N)(=O)=O] | (S)-N1-(3-amino- 2-hydroxypropyl)- 4-(6,7- dihydroimidazo[1, 2-a]pyrimidin- 8(5H)-yl)-3-(1H- tetrazol-5- yl)benzene-1,2- disulfonamide | 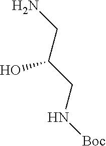 [SMILES: CC(C)(C)OC(=O)NC[C@H](O)CN] | 498 | 499 |

### Example 19

### (R)—N1-(3-amino-2-hydroxypropyl)-4-(2,3-dihydro-1H-imidazo[1,2-a]imidazol-1-yl)-3-(1H-tetrazol-5-yl)benzene-1,2-disulfonamide

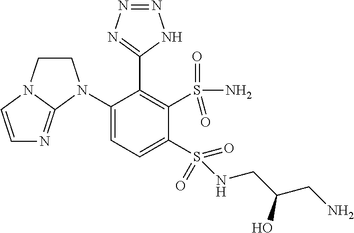 [SMILES: NC[C@@H](O)CNS(=O)(=O)c1ccc(N2CCn3ccnc32)c(-c2nnn[nH]2)c1S(N)(=O)=O]

### Step A: 3-(2,3-dihydro-1H-imidazo[1,2-a]imidazol-1-yl)-N,N-bis(4-methoxybenzyl)-2-(1-(4-methoxybenzyl)-1H-tetrazol-5-yl)-6-((2-(trimethylsilyl)ethyl)sulfonyl)benzenesulfonamide

A mixture of 2,3-dihydro-1H-imidazo[1,2-a]imidazole (0.249 g, 2.283 mmol)] and 3-iodo-N,N-bis(4-methoxybenzyl)-2-(2-(4-methoxybenzyl)-2H-tetrazol-5-yl)-6-((2-(trimethylsilyl)ethyl)sulfonyl)benzenesulfonamide (REFERENCE EXAMPLE 2) (1.0 g, 1.142 mmol), cesium carbonate (1.116 g, 3.43 mmol), and Pd(dppf)Cl₂ (0.186 g, 0.228 mmol) in DME (12 mL) at RT was degassed for 10 min. The reaction mixture was then stirred at 80° C. for 16 h. The mixture was diluted with water (50 mL), and extracted with CH₂Cl₂ (2×50 mL). The combined organic phases were washed with brine, dried (MgSO₄) and concentrated under reduced pressure. The residue was purified by column chromatography on silica gel, eluting with heptane/ethanol, 0-70% to give the title product. LC/MS [M+H]⁺: 857.

### Step B: 2-(N,N-bis(4-methoxybenzyl)sulfamoyl)-4-(2,3-dihydro-1H-imidazo[1,2-a]imidazol-1-yl)-3-(1-(4-methoxybenzyl)-1H-tetrazol-5-yl)benzenesulfinic acid

TBAF (1.283 mL, 1.283 mmol) was added to a stirred solution of 3-(2,3-dihydro-1H-imidazo[1,2-a]imidazol-1-yl)-N,N-bis(4-methoxybenzyl)-2-(1-(4-methoxybenzyl)-1H-tetrazol-5-yl)-6-((2-(trimethylsilyl)ethyl)sulfonyl)benzenesulfonamide (500 mg, 0.583 mmol) in tetrahydrofuran (5 mL) at RT and the mixture was stirred at RT for 1 h. After that time, the solvent was removed. The residue was then dissolved with CHCl₃ (40 mL), and washed with a saturated aqueous solution of KHSO₄ (2×40 mL). The organic phase was washed with brine, dried (MgSO₄) and concentrated under reduced pressure. The residue crude product was used without further purification. LC/MS [M+H]+: 757.

### Step C: tert-butyl (R)-(3-((2-(N,N-bis(4-methoxybenzyl)sulfamoyl)-4-(2,3-dihydro-1H-imidazo[1,2-a]imidazol-1-yl)-3-(1-(4-methoxybenzyl)-1H-tetrazol-5-yl)phenyl)sulfonamido)-2-hydroxypropyl)carbamate

(S)-tert-butyl (3-amino-2-hydroxypropyl)carbamate (138 mg, 0.727 mmol) and TEA (0.152 mL, 1.090 mmol) were added to a stirred solution of starting material 2-(N,N-bis(4-methoxybenzyl)sulfamoyl)-4-(2,3-dihydro-1H-imidazo[1,2-a]imidazol-1-yl)-3-(1-(4-methoxybenzyl)-1H-tetrazol-5-yl)benzenesulfinic acid (275 mg, 0.363 mmol) in CH₂Cl₂ (0.6 mL) at RT. The reaction was cooled to 0° C. and NCS (97 mg, 0.73 mmol) was added to the reaction. The mixture was stirred at 0° C. for 30 min. After that time, the mixture was diluted with water (40 mL), extracted with CH₂Cl₂ (2×30 mL). The combined organic phases were washed with brine, dried (MgSO₄) and concentrated under reduced pressure. The residue was purified by column chromatography on silica gel, eluting with 0-60% heptane/ethanol to give the title compound. LC/MS [M+H]: 945.

### Step D: (R)—N1-(3-amino-2-hydroxypropyl)-4-(2,3-dihydro-1H-imidazo[1,2-a]imidazol-1-yl)-N2-(4-methoxybenzyl)-3-(1-(4-methoxybenzyl)-1H-tetrazol-5-yl)benzene-1,2-disulfonamide

1,4-dimethoxybenzene (175 mg, 1.270 mmol) and TFA (2 mL, 26.0 mmol) were added to a stirred solution of starting material tert-butyl ((2R)-3-(2-(N,N-bis(4-methoxybenzyl)sulfamoyl)-4-(2,3-dihydro-1H-imidazo[1,2-a]imidazol-1-yl)-3-(1-(4-methoxybenzyl)-1H-tetrazol-5-yl)phenylsulfonamido)-2-hydroxypropyl)carbamate (120 mg, 0.127 mmol) in TFA (2 mL) at RT and the mixture was stirred at RT for 1 h. The mixture was concentrated to give the crude product which was used without further purification. LC/MS [M+H]+: 725.

### Step E: (R)—N1-(3-amino-2-hydroxypropyl)-4-(2,3-dihydro-1H-imidazo[1,2-a]imidazol-1-yl)-3-(1H-tetrazol-5-yl)benzene-1,2-disulfonamide

1,4-dimethoxybenzene (88 mg, 0.635 mmol) and TFA (2 mL, 26.0 mmol) were added to a stirred solution of starting material N1-((R)-3-amino-2-hydroxypropyl)-4-(2,3-dihydro-1H-imidazo[1,2-a]imidazol-1-yl)-N2-(4-methoxybenzyl)-3-(1-(4-methoxybenzyl)-1H-tetrazol-5-yl)benzene-1,2-disulfonamide (92 mg, 0.127 mmol) in TFA (2 mL) at RT and the mixture was stirred at 80° C. for 90 min. The mixture was concentrated. The residue was purified by preparative HPLC reverse phase (C18), eluting with 0-25% acetonitrile/water with 0.05% NH₃, to give (R)—N1-(3-amino-2-hydroxypropyl)-4-(2,3-dihydro-1H-imidazo[1,2-a]imidazol-1-yl)-3-(1H-tetrazol-5-yl)benzene-1,2-disulfonamide after lyophilization. LC/MS [M+H]+=485.

EXAMPLE 20 in the Table below was prepared in an analogous fashion to that described for EXAMPLE 19 using the amine shown in the table.

| EX | | | | | LC/MS |
|---|---|---|---|---|---|
| NO | Structure | Name | Amine | MW | [M + H]⁺ |
| 20 | 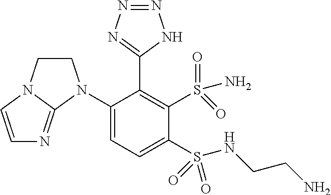 [SMILES: NCCNS(=O)(=O)c1ccc(N2CCn3ccnc32)c(-c2nnn[nH]2)c1S(N)(=O)=O] | N1-(2- aminoethyl)-4- (2,3-dihydro-1H- imidazo[1,2- a]imidazol-1-yl)- 3-(1H-tetrazol-5- yl)benzene-1,2- disulfonamide | 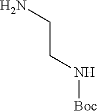 [SMILES: CC(C)(C)OC(=O)NCCN] | 454 | 455 |

### Examples 21-35

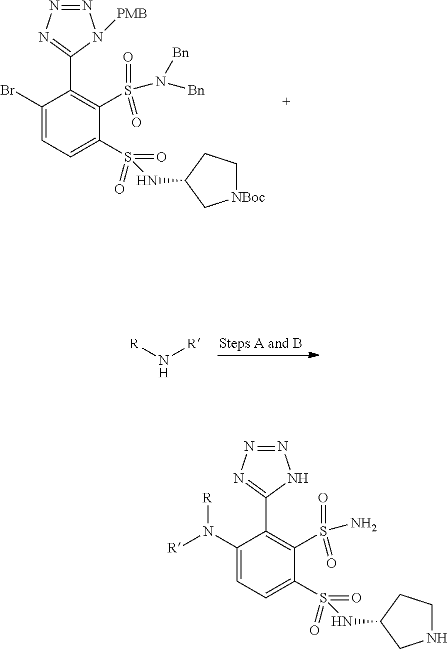

### Parallel Synthesis Examples 21-35

### Step A

In a glove box under a nitrogen atmosphere, to a set of vials each containing the requisite amines (shown in table below) (0.117 mmol), DPPF-Pd-G3 precatalyst (4.3 mg, 4.69 umol), rac-BINAP-Pd-G3 precatalyst (4.7 mg, 4.69 umol) and cesium carbonate (61.1 mg, 0,188 mmol) were added 1.0 mL of a solution of tert-butyl (R)-3-((4-bromo-2-(N,N-dibenzylsulfamoyl)-3-(1-(4-methoxybenzyl)-1H-tetrazol-5-yl)phenyl)sulfonamido)pyrrolidine-1-carboxylate from REFERENCE EXAMPLE 9 (40 mg, 0.047 mmol) in DME. The vials were capped and heated at 85° C. with stirring for 8 hr. After the vials were cooled to room temperature, the reaction mixtures were concentrated under reduced pressure.

### Step B

To the vials containing the residues from Step A were each added DCM 0.4 mL, TFA 0.4 mL and thioanisole (0.3 mL). The vials were shaked at 25° C. for 3 hours. After that time, the solvent was removed under reduced pressure. To each of the vials was added 1 mL TFA and the mixtures were agitated at 55° C. for 4 hours. The reactions were concentrated under reduced pressure. The residues were dissolved in DMSO. Each crude mixture was filtered and the crude products were purified by mass triggered reverse phase HPLC using the following conditions: [column: Waters XBridge C18, or Waters Sunfire C18, 5 μm, 19×100 mm; solvent: gradient range 3-28% initial to 45-95% final MeCN (0.1% TFA) in water (0.1% TFA) 50 or 70 mL/min; 8 min run time] to afford EXAMPLES 21-35.

| | | | | Calc'd | LC/MS |
|---|---|---|---|---|---|
| Ex. | | | | Mass | m/e |
| No. | RR′NH | Structure | Name | [M + H]⁺ | [M + H]⁺ |
| 21 | 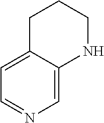 | 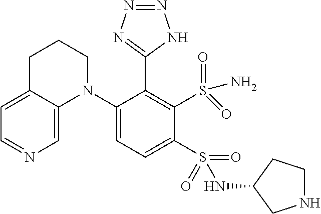 [SMILES: NS(=O)(=O)c1c(S(=O)(=O)N[C@@H]2CCNC2)ccc(N2CCCc3ccncc32)c1-c1nnn[nH]1] | 4-(3,4-dihydro-1,7- naphthyridin-1(2H)-yl)- N1-[(3R)-pyrrolidin-3-yl]- 3-(1H-tetrazol-5- yl)benzene-1,2- disulfonamide | 506.1 | 506.1 |
| 22 | 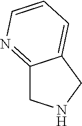 | 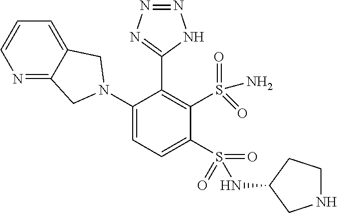 [SMILES: NS(=O)(=O)c1c(S(=O)(=O)N[C@@H]2CCNC2)ccc(N2Cc3cccnc3C2)c1-c1nnn[nH]1] | 4-(5,7-dihydro-6H- pyrrolo[3,4-b]pyridin-6- yl)-N1-[(3R)-pyrrolidin-3- yl]-3-(1H-tetrazol-5- yl)benzene-1,2- disulfonamide | 492.1 | 492.1 |
| 23 | 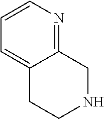 | 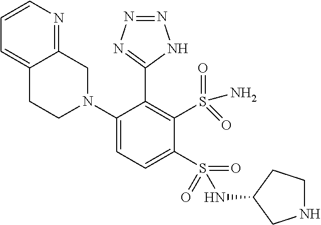 [SMILES: NS(=O)(=O)c1c(S(=O)(=O)N[C@@H]2CCNC2)ccc(N2CCc3cccnc3C2)c1-c1nnn[nH]1] | 4-(5,8-dihydro-1,7- naphthyridin-7(6H)-yl)- N1-[(3R)-pyrrolidin-3-yl]- 3-(1H-tetrazol-5- yl)benzene-1,2- disulfonamide | 506.1 | 506.1 |
| 24 | 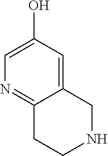 [SMILES: Oc1cnc2c(c1)CNCC2] | 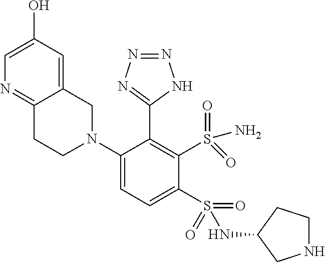 [SMILES: NS(=O)(=O)c1c(S(=O)(=O)N[C@@H]2CCNC2)ccc(N2CCc3ncc(O)cc3C2)c1-c1nnn[nH]1] | 4-(3-hydroxy-7,8- dihydro-1,6-naphthyridin- 6(5H)-yl)-N1-[(3R)- pyrrolidin-3-yl]-3-(1H- tetrazol-5-yl)benzene-1,2- disulfonamide | 522.1 | 522.1 |
| 25 | 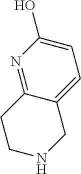 [SMILES: Oc1ccc2c(n1)CCNC2] | 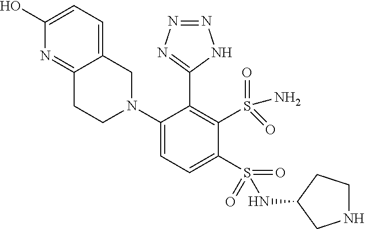 [SMILES: NS(=O)(=O)c1c(S(=O)(=O)N[C@@H]2CCNC2)ccc(N2CCc3nc(O)ccc3C2)c1-c1nnn[nH]1] | 4-(2-hydroxy-7,8- dihydro-1,6-naphthyridin- 6(5H)-yl)-N1-[(3R)- pyrrolidin-3-yl]-3-(1H- tetrazol-5-yl)benzene-1,2- disulfonamide | 522.1 | 522.1 |
| 26 | 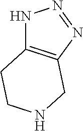 | 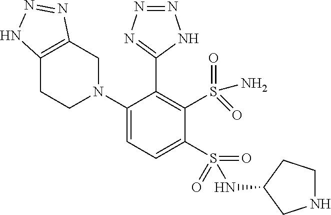 [SMILES: NS(=O)(=O)c1c(S(=O)(=O)N[C@@H]2CCNC2)ccc(N2CCc3[nH]nnc3C2)c1-c1nnn[nH]1] | N1-[(3R)-pyrrolidin-3-yl]- 4-(1,4,6,7-tetrahydro-5H- [1,2,3]triazolo[4,5- c]pyridin-5-yl)-3-(1H- tetrazol-5-yl)benzene-1,2- disulfonamide | 496.1 | 496.1 |
| 27 | 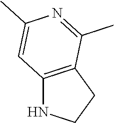 | 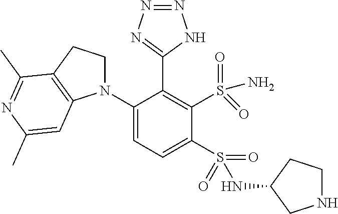 [SMILES: Cc1cc2c(c(C)n1)CCN2c1ccc(S(=O)(=O)N[C@@H]2CCNC2)c(S(N)(=O)=O)c1-c1nnn[nH]1] | 4-(4,6-dimethyl-2,3- dihydro-1H-pyrrolo[3,2- c]pyridin-1-yl)-N1-[(3R)- pyrrolidin-3-yl]-3-(1H- tetrazol-5-yl)benzene-1,2- disulfonamide | 520.2 | 520.2 |
| 28 | 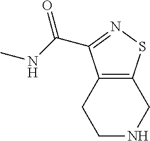 | 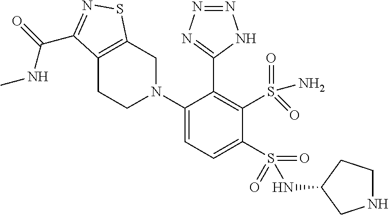 [SMILES: CNC(=O)c1nsc2c1CCN(c1ccc(S(=O)(=O)N[C@@H]3CCNC3)c(S(N)(=O)=O)c1-c1nnn[nH]1)C2] | N-methyl-6-{4-[(3R)- pyrrolidin-3-ylsulfamoyl]- 3-sulfamoyl-2-(1H- tetrazol-5-yl)phenyl}- 4,5,6,7- tetrahydroisothiazolo[5,4- c]pyridine-3-carboxamide | 569.1 | 569.1 |
| 29 | 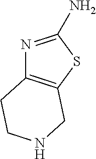 | 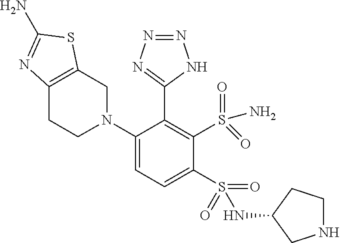 [SMILES: Nc1nc2c(s1)CN(c1ccc(S(=O)(=O)N[C@@H]3CCNC3)c(S(N)(=O)=O)c1-c1nnn[nH]1)CC2] | 4-(2-amino-6,7- dihydro[1,3]thiazolo[5,4- c]pyridin-5(4H)-yl)-N1- [(3R)-pyrrolidin-3-yl]-3- (1H-tetrazol-5- yl)benzene-1,2- disulfonamide | 527.1 | 527.1 |
| 30 | 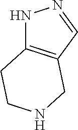 | 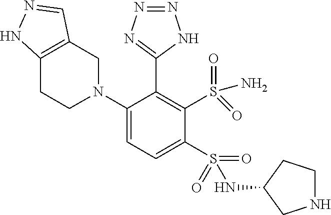 [SMILES: NS(=O)(=O)c1c(S(=O)(=O)N[C@@H]2CCNC2)ccc(N2CCc3[nH]ncc3C2)c1-c1nnn[nH]1] | N1-[(3R)-pyrrolidin-3-yl]- 4-(1,4,6,7-tetrahydro-5H- pyrazolo[4,3-c]pyridin-5- yl)-3-(1H-tetrazol-5- yl)benzene-1,2- disulfonamide | 495.1 | 495.1 |
| 31 | 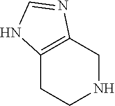 | 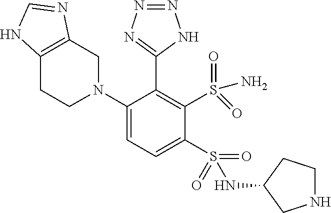 [SMILES: NS(=O)(=O)c1c(S(=O)(=O)N[C@@H]2CCNC2)ccc(N2CCc3[nH]cnc3C2)c1-c1nnn[nH]1] | N1-[(3R)-pyrrolidin-3-yl]- 4-(1,4,6,7-tetrahydro-5H- imidazo[4,5-c]pyridin-5- yl)-3-(1H-tetrazol-5- yl)benzene-1,2- disulfonamide | 495.1 | 495.1 |
| 32 | 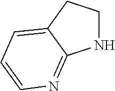 | 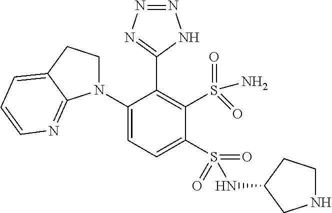 [SMILES: NS(=O)(=O)c1c(S(=O)(=O)N[C@@H]2CCNC2)ccc(N2CCc3cccnc32)c1-c1nnn[nH]1] | 4-(2,3-dihydro-1H- pyrrolo[2,3-b]pyridin-1- yl)-N1-[(3R)-pyrrolidin-3- yl]-3-(1H-tetrazol-5- yl)benzene-1,2- disulfonamide | 492.1 | 492.1 |
| 33 | 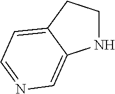 | 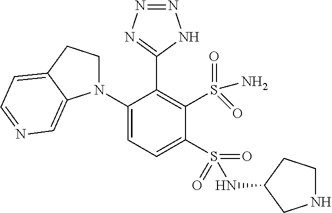 [SMILES: NS(=O)(=O)c1c(S(=O)(=O)N[C@@H]2CCNC2)ccc(N2CCc3ccncc32)c1-c1nnn[nH]1] | 4-(2,3-dihydro-1H- pyrrolo[2,3-c]pyridin-1- yl)-N1-[(3R)-pyrrolidin-3- yl]-3-(1H-tetrazol-5- yl)benzene-1,2- disulfonamide | 492.1 | 492.1 |
| 34 | 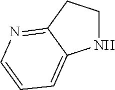 | 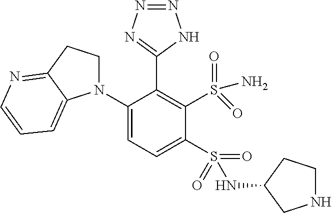 [SMILES: NS(=O)(=O)c1c(S(=O)(=O)N[C@@H]2CCNC2)ccc(N2CCc3ncccc32)c1-c1nnn[nH]1] | 4-(2,3-dihydro-1H- pyrrolo[3,2-b]pyridin-1- yl)-N1-[(3R)-pyrrolidin-3- yl]-3-(1H-tetrazol-5- yl)benzene-1,2- disulfonamide | 492.1 | 492.1 |
| 35 | 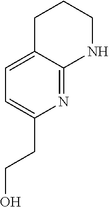 [SMILES: OCCc1ccc2c(n1)NCCC2] | 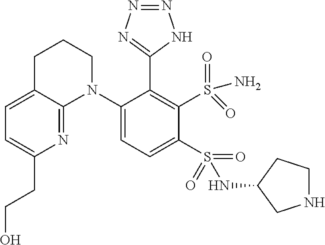 [SMILES: NS(=O)(=O)c1c(S(=O)(=O)N[C@@H]2CCNC2)ccc(N2CCCc3ccc(CCO)nc32)c1-c1nnn[nH]1] | 4-[7-(2-hydroxyethyl)- 3,4-dihydro-1,8- naphthyridin-1(2H)-yl]- N1-[(3R)-pyrrolidin-3-yl]- 3-(1H-tetrazol-5- yl)benzene-1,2- disulfonamide | 550.2 | 550.2 |

### Example 36

### (R)—N1-(3-amino-2-hydroxypropyl)-4-(2,3-dihydro-1H-pyrrolo [2,3-b]pyridin-1-yl)-3-(2H-tetrazol-5-yl)benzene-1,2-disulfonamide

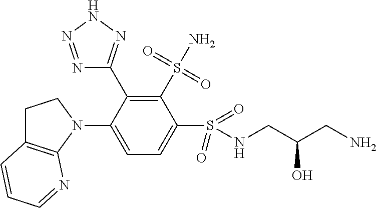 [SMILES: NC[C@@H](O)CNS(=O)(=O)c1ccc(N2CCc3cccnc32)c(-c2nn[nH]n2)c1S(N)(=O)=O]

### Step A: (R)-tert-butyl(3-(2-(N,N-bis(4-methoxybenzyl)sulfamoyl)-4-(2,3-dihydro-1H-pyrrolo[2,3-b]pyridin-1-yl)-3-(2-(4-methoxybenzyl)-2H-tetrazol-5-yl)phenylsulfonamido)-2-((tert-butyldimethylsilyl)ox)propyl)carbamate

To a mixture of (R)-tert-butyl (3-((2-(N,N-bis(4-methoxybenzyl)sulfamoyl)-4-iodo-3-(2-(4-methoxybenzyl)-2H-tetrazol-5-yl)phenyl)sulfonyl)-2-hydroxypropyl)carbamate (REFERENCE EXAMPLE 7) (0.50 g, 0.46 mmol), 2,3-dihydro-H-pyrrolo[2,3-b] pyridine (0.17 g, 1.39 mmol), sodium tert-butoxide (0.13 g, 1.39 mmol) in toluene (5 mL) were added Pd₂dba₃ (85 mg, 0.09 mmol) and X-Phos (88 mg, 0.18 mmol). The mixture was degassed with nitrogen three times. The mixture was stirred for 18 h at 100° C. under nitrogen. The resulting mixture was cooled to room temperature, diluted with water (20 mL) and extracted with EA (3×20 mL). The combined organic layers were washed with brine (2×30 mL), dried over anhydrous Na₂SO₄ and filtered. The filtrate was concentrated under reduced pressure. The residue was purified by a silica gel column chromatography, eluting with 50% EA in PE to afford the title compound. LCMS (ESI) calc'd for C₅₂H₆₇N₉O₁₀S₂Si [M+H]⁺: 1070, found 1070.

### Step B: (R)—N1-(3-amino-2-hydroxypropyl)-4-(2,3-dihydro-1H-pyrrolo[2,3-b]pyridin-1-yl)-3-(2H-tetrazol-5-yl)benzene-1,2-disulfonamide

TFA (5 mL, 65 mmol) was added to (R)-tert-butyl (3-(2-(N,N-bis(4-methoxybenzyl)sulfamoyl)-4-(2,3-dihydro-1H-pyrrolo [2,3-b]pyridin-1-yl)-3-(2-(4-methoxybenzyl)-2H-tetrazol-5-yl)phenylsulfonamido)-2-((tert-butyldimethylsilyl)oxy)propyl)carbamate (55.6 mg, 0.065 mmol), and the reaction mixture was heated to 80° C. for 1 hour. After that time, the reaction mixture was concentrated under reduced pressure to afford the crude product. The crude product was purified by Prep-HPLC with the following conditions: Column: Atlantis Prep T3 OBD Column, 19×250 mm, 10 μm; Mobile Phase A: water (10 mmol/L NH₄HCO₃), Mobile Phase B: ACN; Flow rate: 20 mL/min; Gradient: 10% B to 45% B in 8 min; Detector: UV 254/210 nm; Retention time: 6.83 min to give the title compound. LCMS (ESI) calc'd for C₁₇H₂₁N₉O₅S₂ [M+H]⁺: 496, found 496; ¹H NMR (400 MHz, DMSO-d₆) δ 8.13 (d, J=8.9 Hz, 1H), 8.04 (d, J=8.9 Hz, 1H), 7.82 (m, 1H), 7.39 (m, 1H), 7.16 (br, 3H), 6.67 (m, 1H), 3.76-3.80 (m, 1H), 3.12 (m, 1H), 3.04-2.87 (m, 2H), 2.83-2.68 (m, 5H).

### Example 37

### (R)-6-((3-amino-2-hydroxypropyl)sulfonyl)-3-(6,7-dihydroimidazo[1,2-a]pyrimidin-8(5H)-yl)-2-(2H-tetrazol-5-yl)benzenesulfonamide

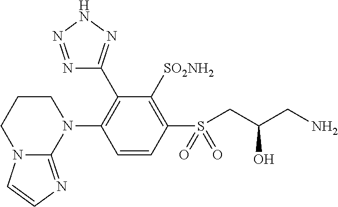 [SMILES: NC[C@@H](O)CS(=O)(=O)c1ccc(N2CCCn3ccnc32)c(-c2nn[nH]n2)c1S(N)(=O)=O]

### Step A: (R)-tert-butyl (3-((2-(N,N-bis(4-methoxybenzyl)sulfamoyl)-4-(6,7-dihydroimidazo[1,2-a]pyrimidin-8(5H)-yl)-3-(2-(4-methoxybenzyl)-2H-tetrazol-5-yl)phenyl)sulfonyl)-2-((tert-butyldimethylsilyl)oxy)propyl)carbamate

To a solution of (R)-tert-butyl (3-((2-(N,N-bis(4-methoxybenzyl)sulfamoyl)-4-iodo-3-(2-(4-methoxybenzyl)-2H-tetrazol-5-yl)phenyl)sulfonyl)-2-((tert-butyldimethylsilyl) oxy)propyl)carbamate (REFERENCE EXAMPLE 16) (0.80 g, 0.75 mmol) in DME (7 mL) were added 5,6,7,8-tetrahydroimidazo[1,2-a]pyrimidine hydrochloride (0.18 g, 1.13 mmol), Cs₂CO₃ (0.74 g, 2.28 mmol) and PdCl₂(dppf) adduct CH₂Cl₂ (0.12 g, 0.15 mmol) at room temperature. The mixture was degassed with nitrogen three times. The reaction mixture was stirred at 80° C. for 16 h under nitrogen. After cooling to room temperature, the resulting mixture was concentrated under vacuum. The residue was purified by a silica gel column chromatography, eluting with 10% MeOH in DCM to afford the title compound. LCMS (ESI) calc'd for C₅₁H₆₇N₉O₁₀S₂Si [M+H]⁺: 1058, found 1058.

### Step B: (R)-6-((3-amino-2-hydroxypropyl)sulfonyl)-3-(6,7-dihydroimidazo[1,2-a]pyrimidin-8(5H)-yl)-2-(2H-tetrazol-5-yl)benzenesulfonamide

The title compound was prepared as described for EXAMPLE 36 Step B using (R)-tert-butyl(3-((2-(N,N-bis(4-methoxybenzyl)sulfamoyl)-4-(6,7-dihydroimidazo [1,2-a]pyrimidin-8(5H)-yl)-3-(2-(4-methoxybenzyl)-2H-tetrazol-5-yl)phenyl)sulfonyl)-2-((tert-butyldimethylsilyl)oxy)propyl)carbamate to afford the crude product. The crude product was purified by Prep-HPLC with the following conditions: Column: XBridge C18 Column 19×250 mm; Mobile Phase A: water (10 mmol/L NH₄HCO₃), Mobile Phase B: ACN; Flow rate: 20 mL/min; Gradient: 3% B to 30% B in 10 min; Detector: UV 254/210 nm; Retention time: 7.6 min to give the title compound. LCMS (ESI) calc'd for C₁₆H₂₁N₉O₅S₂ [M+H]⁺: 484, found 484; ¹H NMR (300 MHz, CD₃OD) δ 8.38 (d, J=8.7 Hz, 1H), 7.92 (d, J=8.7 Hz, 1H), 6.63-6.59 (m, 1H), 6.44-6.40 (m, 1H), 4.30-4.27 (m, 1H), 3.96-3.91 (m, 2H), 3.80 (t, J=6.0 Hz, 2H), 3.20-3.10 (m, 2H), 3.03-2.97 (m, 1H), 2.85-2.75 (m, 1H), 1.78 (m, 2H).

EXAMPLES 38-40 in the table below were prepared in an analogous fashion to that described for EXAMPLE 37, starting from (R)-tert-butyl (3-(2-(N,N-bis(4-methoxybenzyl)sulfamoyl)-4-iodo-3-(2-(4-methoxybenzyl)-2H-tetrazol-5-yl)phenylsulfonamido)-2-((tert-butyldimethylsilyl)oxy)propyl)carbamate (REFERENCE EXAMPLE 7) and the requisite amines, which were prepared as described herein, or which were available from commercial sources.

| | | | | |
|---|---|---|---|---|
| 38 | 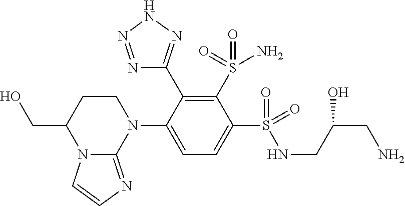 [SMILES: NC[C@@H](O)CNS(=O)(=O)c1ccc(N2CCC(CO)n3ccnc32)c(-c2nn[nH]n2)c1S(N)(=O)=O] | N1-((R)-3-amino-2- hydroxypropyl)-4-(5- (hydroxymethyl)-6,7- dihydroimidazo[1,2- a]pyrimidin-8(5H)-yl)-3- (2H-tetrazol-5- yl)benzene-1,2- disulfonamide | 528 | 529 |
| 39 | 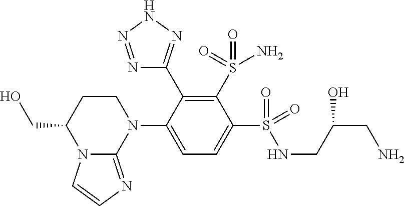 [SMILES: NC[C@@H](O)CNS(=O)(=O)c1ccc(N2CC[C@@H](CO)n3ccnc32)c(-c2nn[nH]n2)c1S(N)(=O)=O] | N1-((R)-3-amino-2- hydroxypropyl)-4-((S)- 5-(hydroxymethyl)-6,7- dihydroimidazo[1,2- a]pyrimidin-8(5H)-yl)- 3-(2H-tetrazol-5- yl)benzene-1,2- disulfonamide | 528 | 529 |
| 40 | 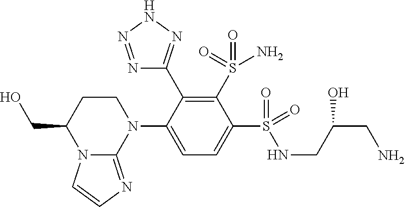 [SMILES: NC[C@@H](O)CNS(=O)(=O)c1ccc(N2CC[C@H](CO)n3ccnc32)c(-c2nn[nH]n2)c1S(N)(=O)=O] | N1-((R)-3-amino-2- hydroxypropyl)-4-((R)- 5-(hydroxymethyl)-6,7- dihydroimidazo[1,2- a]pyrimidin-8(5H)-yl)- 3-(2H-tetrazol-5- yl)benzene-1,2- disulfonamide | 528 | 529 |

### Example 38

¹H NMR (400 MHz, D₂O): δ 8.33 (d, J=8.6 Hz, 1H), 7.87 (d, J=8.6 Hz, 1H), 6.75 (d, J=1.8 Hz, 1H), 6.48 (d, J=1.8 Hz, 1H), 4.03-3.85 (m, 2H), 3.74-3.50 (m, 2H), 3.28-3.01 (m, 5H), 2.90-2.78 (m, 1H), 1.82-1.67 (m, 2H).

### Example 39

1H NMR (300 MHz, D₂O) δ 8.28 (d, J=8.6 Hz, 1H), 7.80 (d, J=8.6 Hz, 1H), 6.70 (d, J=1.8 Hz, 1H), 6.42 (d, J=1.8 Hz, 1H), 3.98-3.77 (m, 2H), 3.69-3.41 (m, 2H), 3.29-2.88 (m, 5H), 2.86-2.66 (m, 1H), 1.80-1.56 (m, 2H).

### Example 40

¹H NMR (300 MHz, D₂O): δ 8.36 (d, J=8.6 Hz, 1H), 7.91 (d, J=8.6 Hz, 1H), 6.77 (d, J=2.1 Hz, 1H), 6.55 (d, J=2.1 Hz, 1H), 4.08-3.84 (m, 2H), 3.72-3.43 (m, 2H), 3.38-2.91 (m, 5H), 2.95-2.69 (m, 1H), 1.88-1.41 (m, 2H).

### Example 41

### Synthesis of: (R)—N1-(3-amino-2-hydroxypropyl)-4-(1H-imidazol-1-yl)-3-(2H-tetrazol-5-yl)benzene-1,2-disulfonamide

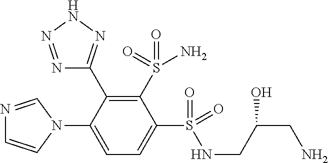 [SMILES: NC[C@@H](O)CNS(=O)(=O)c1ccc(-n2ccnc2)c(-c2nn[nH]n2)c1S(N)(=O)=O]

### Step A: (R)-tert-butyl (2-((tert-butyldimethylsilyl)oxy)-3-(2-(N,N-dibenzylsulfamoyl)-4-(1H-imidazol-1-yl)-3-(2-(4-methoxybenzyl)-2H-tetrazol-5-yl)phenylsulfonamido) propyl)carbamate

To a solution of (R)-tert-butyl (3-(4-bromo-2-(N,N-dibenzylsulfamoyl)-3-(2-(4-methoxybenzyl)-2H-tetrazol-5-yl)phenylsulfonamido)-2-((tert-butyldimethylsilyl) oxy)propyl)carbamate (REFERENCE EXAMPLE 10) (1.30 g, 1.34 mmol) in DMSO (3 mL) were added 1H-imidazole (0.273 g, 4.017 mmol), pyrrolidine-2-carboxylic acid (0.231 g, 2.01 mmol), copper(I) iodide (0.254 g, 1.34 mmol) and K₃PO₄ (0.851 g, 4.017 mmol) at room temperature. The mixture was degassed with nitrogen for three times. The reaction mixture was stirred at 100° C. for 16 h under nitrogen. After cooling to room temperature, the resulting mixture was filtered and the filtrate was concentrated under reduced pressure. The residue was purified by silica gel column chromatography, eluting with a gradient of 1%-70% EA in PE to afford the title compound. LCMS (ESI) calc'd for C₄₆H₅₉N₉O₈S₂Si [M+1]⁺: 958, found 958.

### Step B: (R)-tert-butyl (2-((tert-butyldimethylsilyl)oxy)-3-(2-(N,N-dibenzylsulfamoyl)-4-(1H-imidazol-1-yl)-3-(2-(4-methoxybenzyl)-2H-tetrazol-5-yl)phenylsulfonamido) propyl)carbamate

A mixture of (R)-tert-butyl (2-((tert-butyldimethylsilyl)oxy)-3-(2-(N,N-dibenzylsulfamoyl)-4-(1H-imidazol-1-yl)-3-(2-(4-methoxybenzyl)-2H-tetrazol-5-yl)phenylsulfonamido) propyl)carbamate (0.190 g, 0.198 mmol) in TFA (2 mL) was stirred at RT for 1 hour. After that time the reaction mixture was concentrated under reduced pressure. The resultant residue was co-evaporated with anisole (3×2 mL) under reduced pressure. The crude residue was then treated with H₂SO₄ (0.5 mL) at 0° C. for 1 hour. To the reaction mixture was added water (1 mL) and the resultant reaction mixture was heated to 80° C. with stirring for 1 hour. To the reaction mixture was added EtOAc (10 mL) and the mixture was extracted with water (3×5 mL). The combined aqueous layers were adjusted to approximately pH 6-7 with ammonium hydroxide. The water was removed under reduced pressure. Methanol was added to the residue and the mixture was filtered to remove solids and the filtrate was concentrated to afford a crude residue. The crude product was purified by Prep-HPLC with the following conditions: Column: XBridge Prep C18 OBD Column 100A, 19×250 mm, 10 μm; Mobile Phase A: water (10 mmoL/L NH₄HCO₃+0.1% NH₄OH), Mobile Phase B: ACN; Flow rate: 20 mL/min; Gradient: 2% B to 4% B in 15 min; Detector: UV 254 nm and 220 nm; Retention time: 12.8 min to afford the title compound. LCMS (ESI) calc'd for C₁₃H₁₇N₉O₅S₂ [M+1]⁺: 444, found 444; ¹H NMR (400 MHz, CD₃D) δ 8.53 (d, J=8.5 Hz, 1H), 7.90 (d, J=8.5 Hz, 1H), 7.45-7.42 (m, 1H), 6.88-6.85 (m, 2H), 3.89-3.80 (m, 1H), 3.25-3.09 (m, 2H), 3.00-2.98 (m, 1H), 2.83-2.81 (m, 1H).

### Biological Assays

### Enzyme Activity: Determination of IC₅₀

The Class B enzyme activities were measured in the presence of the test inhibitor in a fluorescence assay against a commercially available substrate consisting of a cephalosporin core linking 7-hydroxycoumarin to fluorescein (CCF2-FA). The enzyme (NDM-1, IMP-1 or VIM-1) and the substrate were diluted in 100 mM KH₂PO₄ buffer (pH 7) containing 0.005% Tween-20 and 10 μM ZnSO₄. In the assay, the final concentration of enzyme was 1 pM, 2 pM and 30 pM for NDM-1, IMP-1 and VIM-1, respectively, and the final concentration of CCF2-FA was 1.25 μM. The test inhibitor was dissolved in dimethylsulfoxide and diluted 1:50 in the assay, resulting in a final concentration range of 20 μM to 0.00063 μM. In a 384-well microplate, the test inhibitor was incubated with the metallo-β-lactamase enzyme and the substrate for 2 hours at 25° C. Fluorescence at 460 nm following excitation at 405 nm was measured. The IC₅₀ value was determined from semi-logarithmic plots of enzyme inhibition versus inhibitor concentration, with a curve generated using a 4-parameter fit.

Representative compounds of the present invention exhibit inhibition of Class B β-lactamases in this assay. For example, the compounds of Examples 1-41 were tested in this assay and were found to have the IC₅₀ values shown in Table 1.

### Antibiotic Potentiation Activity: Determination of Synergistic Concentration

The concentrations of metallo-β-lactamase inhibitors required to restore the susceptibility of various strains of bacteria to inactive concentrations of antibiotics were determined in an assay that assessed bacterial growth by measuring the optical density at 600 nm (OD₆₀₀). The bacterial strains tested included the clinical strains Escherichia coli expressing NDM-1 (CLB30005, CLB30016), Serratia marcescens expressing IMP-1 (CL5741), and Klebsiella pneumoniae expressing VIM-1 (IHMA599644). Inhibitor activity was measured in the presence and absence of imipenem in a 384-well microplate.

The clinical strains CLB30016, CL5741 and IHMA599644 were grown on trypticase soy agar containing 5% sheep's blood. The bacteria on agar plates were incubated at 35° C. with humidity overnight. The following day, individual colonies from each clinical strain were picked and resuspended in 5 mL saline to attain an OD₆₀₀ of 0.14, 0.11, 0.15 and 0.13, for CLB30016, CL5741 and IHMA599644, respectively. These were further diluted 1:100 into 1.1× CAMHB and used to inoculate the test wells as described below.

Imipenem in 10 mM 3-(N-morpholino)propanesulfonic acid (MOPS, pH 7) was stored in single use aliquots at −80° C. Test inhibitors were dissolved in dimethylsulfoxide and diluted 1:50 in the assay, resulting in a final concentration range of 200 μM to 0.195 μM. On the day of the assay, 4 μl of antibiotic was added to 45 μl of bacteria followed by 1 μl of test compound and mixed by pipetting and with an orbital shaker. The concentration of antibiotic used in the assay was 1 μg/mL. Microplates were covered and incubated at 35° C. for 22 hours to 24 hours. At the end of the incubation, absorbance was determined using a spectrophotometer. The synergistic concentration of MBLI was determined by identifying the lowest concentration of test compound in the presence of a given concentration of antibiotic that was required to inhibit 95% of the growth of the bacteria. The results for Examples 1-209 are reported in Table 1, expressed as the concentration of compound that potentiated the action of antibiotic (imipenem) affecting 95% inhibition of bacterial growth (MITC95).

Representative compounds of the present invention do not have any or have minimal intrinsic antibacterial activity but display a synergistic effect when used in combination with a beta-lactam antibiotic. For example, in general, the compounds of Examples 1-209 were determined to restore susceptibility to imipenem for one or more of the test organisms at concentrations of 100 μM or less.

TABLE 1
Inhibition of metallo-P-lactamases (IMP-1, NDM-1, VIM-1, VIM-2)
and antibiotic potentiation vs. MBL-expressing bacteria by Examples 1-41.

| | | | | | CLB30016 | CL5741 | IHMA599644 |
|---|---|---|---|---|---|---|---|
| | | | | | E.coli | S.marcescens | K.pneumoniae |
| | NDM1 | IMP1 | VIM1 | VIM2 | MITC95 | MITC95 | MITC95 |
| Ex. | IC₅₀ | IC₅₀ | IC₅₀ | IC₅₀ | NDM | uM IMP | uM VIM |
| No. | (nM) | (nM) | (nM) | (nM) | (μM) | (μM) | (μM) |
| 1 | 1.57 | 4.03 | 0.29 | 0.99 | 3.13 | 1.56 | 6.25 |
| 2 | 0.30 | 2.01 | 0.16 | 0.68 | 0.20 | 0.78 | 1.56 |
| 3 | 2.23 | 5.72 | 22.57 | 72.77 | 0.78 | 3.13 | 6.25 |
| 4 | 2.25 | 2.18 | 0.35 | 0.48 | 3.13 | 6.25 | 6.25 |
| 5 | 0.04 | 0.96 | 0.12 | 0.55 | 12.50 | 6.25 | 12.50 |
| 6 | 0.72 | 6.80 | 0.24 | 1.13 | 0.78 | 3.13 | 6.25 |
| 7 | 0.14 | 2.81 | 0.13 | 0.66 | 6.25 | 3.13 | 6.25 |
| 8 | 0.47 | 6.63 | 0.20 | 1.09 | 3.13 | 6.25 | 12.50 |
| 9 | 0.48 | 2.17 | 0.10 | 0.49 | 0.39 | 0.20 | 1.56 |
| 10 | 1.17 | 2.96 | 0.25 | 0.99 | 0.10 | 0.39 | 1.56 |
| 11 | 1.95 | 4.55 | 1.26 | 3.71 | 0.20 | 0.78 | 1.56 |
| 12 | 1.58 | 4.69 | 0.60 | 2.17 | 0.05 | 0.20 | 0.39 |
| 13 | 0.77 | 9.70 | 0.39 | 2.16 | 0.20 | 0.39 | 0.78 |
| 14 | 1.61 | 6.32 | 0.97 | 2.60 | 0.10 | 0.39 | 1.56 |
| 15 | 2.35 | 8.90 | 1.00 | 2.66 | 0.20 | 0.39 | 1.56 |
| 16 | 1.20 | 5.93 | 0.38 | 1.01 | 0.10 | 0.39 | 1.56 |
| 17 | 0.36 | 2.17 | 0.22 | 1.11 | 0.78 | 0.78 | 1.56 |
| 18 | 1.87 | 8.07 | 0.76 | 1.99 | 0.10 | 0.39 | 1.56 |
| 19 | 1.03 | 12.10 | 5.96 | 9.80 | 0.20 | 0.78 | 3.13 |
| 20 | 1.70 | 9.68 | 12.43 | 15.12 | 0.39 | 0.78 | 3.13 |
| 21 | 0.21 | 2.46 | 0.22 | 1.24 | 0.20 | 0.78 | 6.25 |
| 22 | 0.62 | 0.17 | 0.59 | 2.31 | 0.78 | 3.13 | 6.25 |
| 23 | 24.27 | 43.91 | 21.03 | 39.02 | 3.13 | 12.50 | 12.50 |
| 24 | 0.28 | 1.39 | 0.19 | 0.74 | 0.39 | 1.56 | 3.13 |
| 25 | 3.09 | 6.11 | 1.10 | 4.79 | 0.39 | 0.78 | 1.56 |
| 26 | 2.14 | 5.97 | 1.75 | 5.08 | 0.78 | 1.56 | 3.13 |
| 27 | 5.00 | 84.02 | 0.14 | 1.37 | 1.56 | 6.25 | 3.13 |
| 28 | 0.21 | 0.28 | 0.15 | 0.48 | 0.78 | 1.56 | 6.25 |
| 29 | 6.28 | 7.11 | 8.27 | 32.84 | 1.56 | 1.56 | 12.50 |
| 30 | 0.19 | 1.11 | 0.25 | 0.98 | 0.78 | 1.56 | 6.25 |
| 31 | 1.13 | 7.00 | 0.59 | 3.30 | 0.20 | 0.78 | 3.13 |
| 32 | 0.21 | 1.14 | 0.17 | 0.39 | 1.56 | 1.56 | 3.13 |
| 33 | 0.30 | 1.73 | 0.26 | 0.72 | 1.56 | 3.13 | 3.13 |
| 34 | 0.29 | 5.21 | 0.25 | 0.81 | 0.39 | 1.56 | 3.13 |
| 35 | 4.34 | 4.68 | 5.78 | 5.59 | 0.78 | 1.56 | 6.25 |
| 36 | 0.36 | 1.40 | 0.27 | 0.49 | 1.56 | 1.56 | 3.13 |
| 37 | 4.31 | 5.60 | 2.22 | 4.44 | 0.20 | 0.78 | 1.56 |
| 38 | 0.64 | 4.17 | 0.40 | 2.42 | 0.20 | 0.78 | 1.56 |
| 39 | 0.89 | 6.52 | 1.62 | 14.33 | 0.29 | 0.78 | 1.56 |
| 40 | 1.15 | 5.00 | 0.27 | 2.09 | 0.10 | 0.39 | 1.56 |
| 41 | 0.66 | 2.97 | 1.05 | 0.96 | 0.78 | 0.78 | 1.56 |

### Efflux

In order to assess the contribution of efflux to lack of whole cell inhibition of metallo-beta-lactamase inhibitors of formula I, tool strains were constructed. The strain background is Pseudomonas aeruginosa PAO1. A wild-type (MB5919) and an isogenic strain in which multiple efflux pumps have been disrupted genetically were used. The MBL IMP-1, obtained from a clinical isolate was introduced into the strain pair by the following process:

Plasmid DNA (encoding IMP-1) was extracted from CL 5673 (IMP-1, P. aeruginosa clinical strain) by standard techniques. The plasmid DNA was transformed into parental MB5919 (oprD+, efflux+, inducible AmpC) and MB5890 (oprD+, efflux−, inducible AmpC) isogenic strains by electroporation. These transformed strains were plated onto cation-adjusted Muller-Hinton agar plates containing ceftazidime at 32 μg/mL (MB5919) and 16 μg/mL (MB5890) to select for those cells in which the IMP-1-expressing plasmid was introduced successfully, resulting in resistance to ceftazidime. Agarose-gel electrophoresis of PCR product for IMP-1 from the successful transformants was used to compare to control and to the original strain from which the plasmid was obtained, confirming transfer of the IMP-1 gene (data not shown).

Minimum inhibitory concentrations of sentinel antibiotics were performed to quality control the new strains. The imipenem MIC went up dramatically, as expected, due to presence of the IMP-1, also meropenem (MEM) and ceftazidime (CAZ). The efflux +/− set behaved similarly with non-BL antibiotics as they should with the efflux-strain exhibiting increased sensitivity to chloramphenicol (CAM) and ciprofloxacin (Cipro).

MIC g/mL

| | | | CL 5673 | | pFLp- | | | |
|---|---|---|---|---|---|---|---|---|
| | | | (IMP-1) | | Vim1 | | | |
| | | | plasmid | | plasmid | | pFLp- | |
| | MB | MB | MB | MB | MB | MB | Vim2 | |
| | 59.19 | 5890 | 9798 | 9799 | 9861 | 9862 | plasmid | |
| OprD | OprD+ | OprD+ | OprD+ | OprD+ | OprD+ | OprD+ | OprD+ | OprD+ |
| Efflux | efflux+ | efflux− | efflux+ | efflux− | efflux+ | efflux− | efflux+ | efflux− |
| | MB5919 | MB | MB5919 | MB | MB5919 | MB | MB | MB |
| | | 5890 | Trans | 5890 | Trans | 5890 | 5919 | 5890 |
| | | | IMP1 | Trans | pFlp- | trans | Trans | Trans |
| | | | plasmid | IMP1 | Vim1 | pFlp- | pFlp- | pFlp- |
| | | | | plasmid | plasmid | Vim1 | Vim2 | Vim2 |
| | | | | | | | plasmid | plasmid |
| Imipenem | 4 | 2 | 64 | 32 | >64 | 64 | >64 | 32 |
| Meropenem | 2 | 0.5 | >64 | 64 | >64 | 64 | >64 | 32 |
| Pipercillin | 2 | 1 | 4 | 4 | >256 | 128 | >256 | 128 |
| Chloram- | >64 | 1 | >64 | 1 | >64 | 2 | >64 | 1 |
| phenicol | | | | | | | | |
| Ciprofloxicin | 0.5 | 0.008 | 0.5 | 0.008 | 1 | 0.008 | 1 | 0.008 |
| CAZ | 1 | 0.5 | 256 | 256 | >256 | >256 | 128 | 64 |
| Azithromycin | 16 | 1 | 2 | 2 | 32 | 1 | 32 | 1 |

The strain set was then used as a pair to determine the effect of metallo-β-lactamase inhibitors of Formula Ion the MIC of imipenem and/or ceftazidime. A fixed concentration of antibiotic is included in standard microbroth MIC tests, usually at the CLSI breakpoint concentration. A fixed amount of a class A/C beta-lactamase inhibitor is also included to inhibit the resident Pseudomonas AmpC enzyme. A serial titration of the metallo-β-lactamase inhibitor was included and the concentration of metallo-β-lactamase inhibitor which restores susceptibility of the strain to the included antibiotic was recorded. That concentration of metallo-β-lactamase inhibitor was then compared between the two strains to determine the fold difference between the efflux+(MB9798) and efflux−(MB9799) strains, which indicates n the extent to which the MBLi is subject to efflux.

TABLE 2
Concentration of metallo-β-lactamase inhibitors of Formula I which
restores susceptibility of efflux + (MB9798) and efflux-(MB9799)
strains to imipenem at 2 μg/mL in the presence of a class A, C, D
serine β-lactamase inhibitor closely related to relebactam.

| | | MB9799 | |
|---|---|---|---|
| | MB9798 MITC95 | MITC95 | |
| | P. aeruginosa | P. aeruginosa | |
| Ex | Efflux WT | Efflux Mutant | |
| number | (μM) | (μM) | Efflux ratio |
| 1 | 6.25 | 1.56 | 4.00 |
| 2 | 3.13 | 0.78 | 4.00 |
| 3 | 6.25 | 1.56 | 4.00 |
| 4 | 25.00 | 3.13 | 8.00 |
| 5 | 6.25 | 3.13 | 2.00 |
| 6 | 3.13 | 3.13 | 1.00 |
| 7 | 3.13 | 1.56 | 2.00 |
| 8 | 3.13 | 6.25 | 0.50 |
| 9 | 1.56 | 0.78 | 2.00 |
| 10 | 3.13 | 1.56 | 2.00 |
| 11 | 2.34 | 1.17 | 2.00 |
| 12 | 0.78 | 0.20 | 4.00 |
| 13 | 1.56 | 0.78 | 2.00 |
| 14 | 3.13 | 0.78 | 4.00 |
| 15 | 3.13 | 0.78 | 4.00 |
| 16 | 3.13 | 0.78 | 4.00 |
| 17 | 1.56 | 0.78 | 2.00 |
| 18 | 3.13 | 0.78 | 4.00 |
| 19 | 6.25 | 1.56 | 4.00 |
| 20 | 6.25 | 0.78 | 8.00 |
| 21 | 3.13 | 1.56 | 2.00 |
| 22 | 3.13 | 1.56 | 2.00 |
| 23 | 50.00 | 6.25 | 8.00 |
| 24 | 25.00 | 3.13 | 8.00 |
| 25 | 6.25 | 0.78 | 8.00 |
| 26 | 6.25 | 0.78 | 8.00 |
| 27 | 50.00 | 3.13 | 16.00 |
| 28 | 12.50 | 3.13 | 4.00 |
| 29 | 25.00 | 0.78 | 32.00 |
| 30 | 6.25 | 1.56 | 4.00 |
| 31 | 3.13 | 0.78 | 4.00 |
| 32 | 3.13 | 1.56 | 2.00 |
| 33 | 6.25 | 1.56 | 4.00 |
| 34 | 12.50 | 1.56 | 8.00 |
| 35 | 50.00 | 0.78 | 64.00 |
| 36 | 12.50 | 0.78 | 16.00 |
| 37 | 3.13 | 0.78 | 4.00 |
| 38 | 1.56 | 0.78 | 2.00 |
| 39 | 2.34 | 1.17 | 2.00 |
| 40 | 1.56 | 0.78 | 2.00 |
| 41 | 1.56 | 0.78 | 2.00 |

1. A compound of Formula I 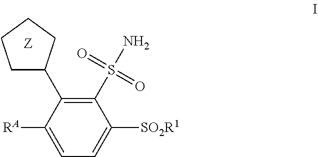
or a pharmaceutically acceptable salt thereof,
wherein:
R^{A} is 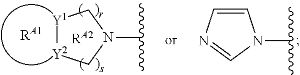 [symbol]
indicates the point of attachment to the rest of the compound,
R^{A1} is a five or six-membered heteroaromatic ring containing 1-3 ring heteroatoms which are independently N, O, or S;
R^{A2} is a five or six-membered heterocyclenyl ring containing 1 or 2 N heteroatoms;
the subscripts r and s are independently 0, 1, 2, or 3, wherein the sum of the subscripts r and s is 2 or 3;
Y¹ and Y² are independently N or C;
wherein R^{A} is optionally substituted with one to three substituents, independently selected from:
(a) —NR^{a}R^{b},
(b) —OR^{a},
(c) —(CH₂)₁₋₃OH,
(d) —C₁-C₆ alkyl, optionally substituted by one to three —F,
(e) —C(O)NR^{a}R^{b}, and
(f) —C(O)OC₁-C₃ alkyl,
Z is tetrazolyl, wherein Z is linked through a carbon to carbon bond to the illustrated phenyl ring;
R¹ is
(1) —NR^{a}—C₁-C₆alkyl, optionally substituted with 1, 2, 3, or 4 substituents independently selected from: —F, —CF₃, —C₁-C₆alkyl, —CH(NH₂)C(O)NH₂, —C(O)NR^{a}R^{b}, —C(O)OH, —(CH₂)₁₋₂NH₂, —NR^{a}(CH₂)₂₋₃NH₂, NR^{a}R^{b}, —N⁺R^{a}R^{b}CH₃, —NHCH₂CH₂OCH₃, —OR^{a}, and —O(CH₂)₂₋₃NH₂;
(2) —C₁-C₆alkyl, optionally substituted with 1, 2, or 3 substituents independently selected from: —F, —CF₃, —C₁-C₆alkyl, —CH(NH₂)C(O)NH₂, —C(O)NR^{a}R^{b}, —C(O)OH, —(CH₂)₁₋₂NH₂, —NR^{a}(CH₂)₂₋₃NH₂, NR^{a}R^{b}, —N⁺R^{a}R^{b}CH₃, —NHCH₂CH₂O CH₃, —OR^{a}, and —O(CH₂)₂₋₃NH₂; or
(3) —NR^{a}—(CH₂)ₙ-HetB1;
HetB1 is a 4-6 membered monocyclic, heterocycloalkyl ring with 1 or 2 heteroatom ring atoms independently selected from N, O and S, wherein a N ring atom is optionally in the form of a quaternary amine, and wherein the ring is optionally substituted with 1 or 2 substituents independently selected from —F, —C₁-C₆ alkyl, —C₁-C₆ hydroxyalkyl, —C(O)OR^{a}, —(CH₂)ₖNR^{a}R^{b}, —OR^{a}, and oxo;
R^{a} and R^{b} are independently H or —C₁-C₆ alkyl;
subscript k is 0, 1, 2, 3, or 4; and
each subscript n is independently 0, 1, or 2.

2. The compound of claim 1, or a pharmaceutically acceptable salt thereof, wherein the compound of Formula I has the Formula IA

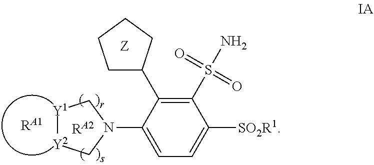

3. The compound of claim 2, or a pharmaceutically acceptable salt thereof, wherein the R^{A} is selected from the group consisting of: 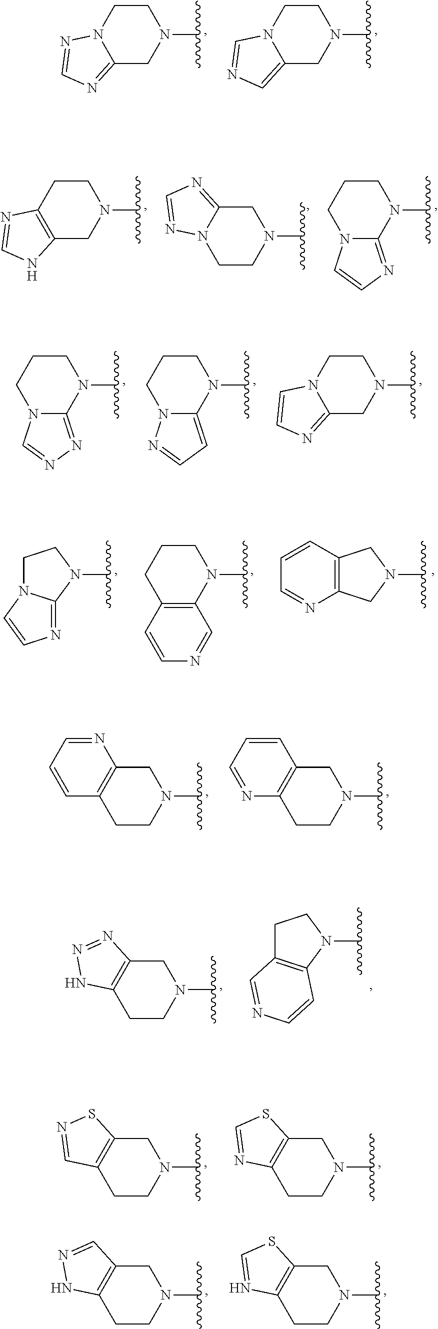 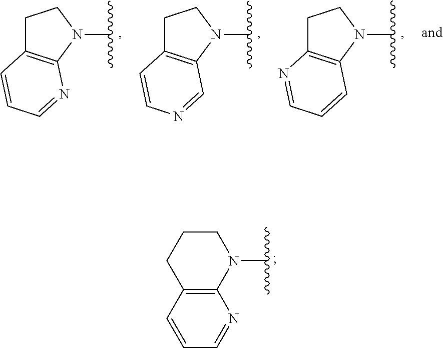
wherein
R^{A} is optionally substituted as set forth in claim 1.

4. The compound of claim 1 or a pharmaceutically acceptable salt thereof, wherein R^{A} is and 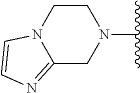 [SMILES: *N1CCn2ccnc2C1]
is optionally substituted as set forth in claim 1.

5. The compound of claim 1, or a pharmaceutically acceptable salt thereof, wherein R¹ is
a) —N(H)—C₂-C₄alkyl, optionally substituted with 1, 2, or 3, substituents independently selected from: —NH₂ and OH;
b) —C₂-C₄alkyl, optionally substituted with 1, 2, or 3, substituents independently selected from —NH₂ and OH; or
c) —N(H)-HetB1, wherein HetB1 is azetidinyl, pyrrolidinyl, or piperidinyl;

6. The compound of claim 5, or a pharmaceutically acceptable salt thereof, wherein R¹ is 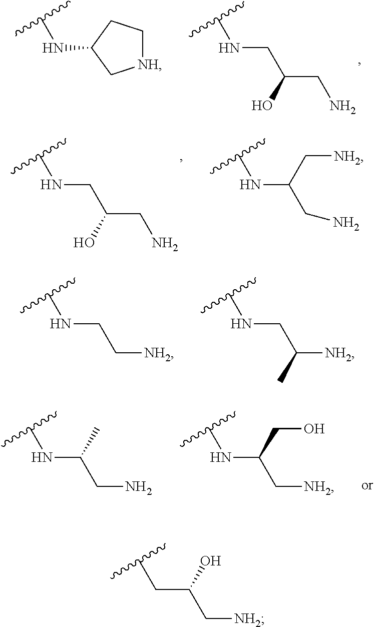
wherein [symbol] indicates point of attachment to the rest of the compound.

7. (canceled)

8. The compound of claim 1 having the structure: 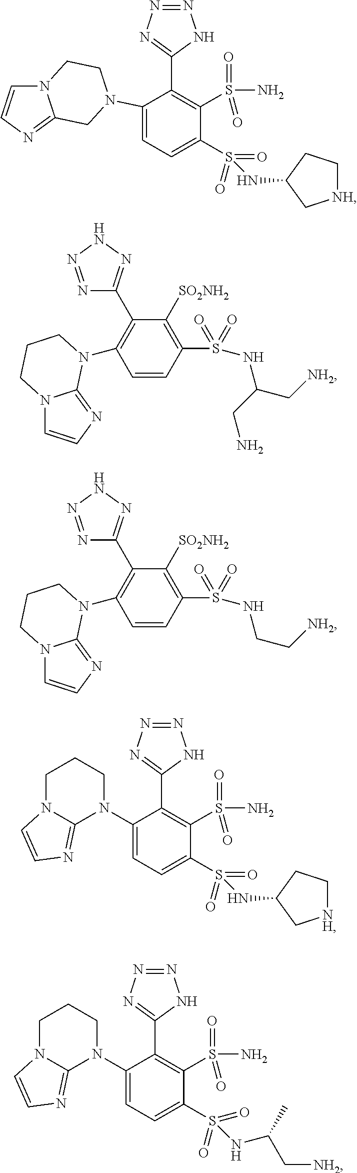 [SMILES: NS(=O)(=O)c1c(S(=O)(=O)N[C@@H]2CCNC2)ccc(N2CCn3ccnc3C2)c1-c1nnn[nH]1 ; NCC(CN)NS(=O)(=O)c1ccc(N2CCCn3ccnc32)c(-c2nn[nH]n2)c1S(N)(=O)=O ; NCCNS(=O)(=O)c1ccc(N2CCCn3ccnc32)c(-c2nn[nH]n2)c1S(N)(=O)=O ; NS(=O)(=O)c1c(S(=O)(=O)N[C@@H]2CCNC2)ccc(N2CCCn3ccnc32)c1-c1nnn[nH]1 ; C[C@H](CN)NS(=O)(=O)c1ccc(N2CCCn3ccnc32)c(-c2nnn[nH]2)c1S(N)(=O)=O] 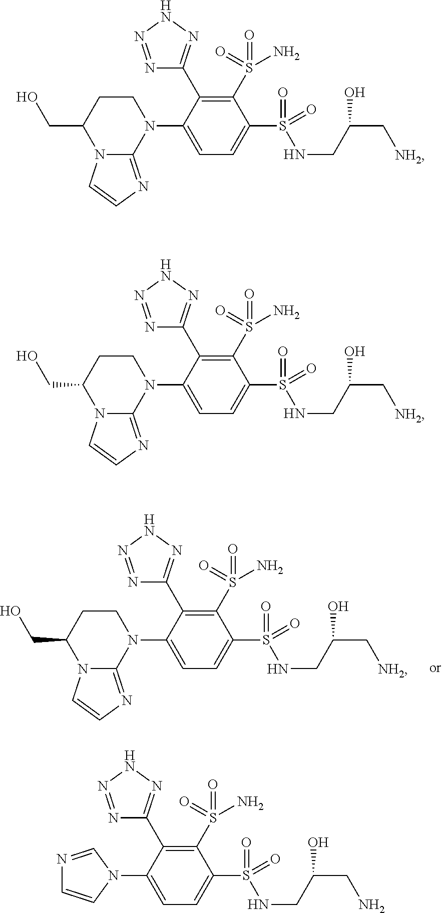 [SMILES: NC[C@@H](O)CNS(=O)(=O)c1ccc(N2CCC(CO)n3ccnc32)c(-c2nn[nH]n2)c1S(N)(=O)=O ; NC[C@@H](O)CNS(=O)(=O)c1ccc(N2CC[C@@H](CO)n3ccnc32)c(-c2nn[nH]n2)c1S(N)(=O)=O ; NC[C@@H](O)CNS(=O)(=O)c1ccc(N2CC[C@H](CO)n3ccnc32)c(-c2nn[nH]n2)c1S(N)(=O)=O ; "or" ; NC[C@@H](O)CNS(=O)(=O)c1ccc(-n2ccnc2)c(-c2nn[nH]n2)c1S(N)(=O)=O]
or a pharmaceutically acceptable salt thereof.

9. A pharmaceutical composition which comprises a compound according to claim 1, or a pharmaceutically acceptable salt thereof, and a pharmaceutically acceptable carrier.

10. The pharmaceutical composition according to claim 9, which further comprises an effective amount of a beta-lactam antibiotic.

11. The pharmaceutical composition according to claim 10 which further comprises an effective amount of one or more beta-lactamase inhibitor compounds.

12. The pharmaceutical composition according to claim 11, wherein the beta-lactamase inhibitor compound is selected from the group consisting of: relebactam or a pharmaceutically acceptable salt thereof, avibactam or a pharmaceutically acceptable salt thereof, vaborbactam or a pharmaceutically acceptable salt thereof, tazobactam or a pharmaceutically acceptable salt thereof, sulbactam or a pharmaceutically acceptable salt thereof, and clavulanic acid or a pharmaceutically acceptable salt thereof.

13. The pharmaceutical composition according to claim 12, wherein the beta-lactamase inhibitor compound is tazobactam or a pharmaceutically acceptable salt thereof and the beta-lactam antibiotic is ceftolozane or a pharmaceutically acceptable salt thereof.

14. The pharmaceutical composition according to claim 12, wherein the beta-lactamase inhibitor compound is relebactam or a pharmaceutically acceptable salt thereof.

15. The pharmaceutical composition according to claim 10, wherein the beta-lactam antibiotic is selected from the group consisting of: (a) imipenem, (b) ertapenem, (c) meropenem, (d) doripenem, (e) biapenem, (f) panipenem, (g) ticarcillin, (h) ampicillin, (i) amoxicillin, (j) carbenicillin, (k) piperacillin, (l) azlocillin, (m) mezlocillin, (n) cefoperazone, (o) cefotaxime, (p) ceftriaxone, (q) cefipime, (r) ceftolozane, (s) ceftazidime, and (t) a pharmaceutically acceptable salt of any of (a) through (s).

16. The pharmaceutical composition according to claim 10, wherein the beta-lactam antibiotic is imipenem or a pharmaceutically acceptable salt thereof.

17. The pharmaceutical composition according to claim 16, further comprising cilastatin or a pharmaceutically acceptable salt thereof.

18. (canceled)

19. A method for treating a bacterial infection which comprises administering to a subject in need of such treatment a therapeutically effective amount of a compound according to claim 1, or a pharmaceutically acceptable salt thereof, in combination with a beta-lactam antibiotic.

20. (canceled)

21. The method of claim 19, wherein the beta-lactam antibiotic is selected from the group consisting of: (a) imipenem, (b) ertapenem, (c) meropenem, (d) doripenem, (e) biapenem, (f) panipenem, (g) ticarcillin, (h) ampicillin, (i) amoxicillin, (j) carbenicillin, (k) piperacillin, (l) azlocillin, (m) mezlocillin, (n) cefoperazone, (o) cefotaxime, (p) ceftriaxone, (q) cefipime, (r) ceftolozane, (s) ceftazidime, and (t) a pharmaceutically acceptable salt of any of (a) through (s).

22. The method of claim 19, wherein the beta-lactam antibiotic is imipenem or a pharmaceutically acceptable salt thereof.

23. A method for treating a bacterial infection which comprises administering to a subject in need of such treatment a therapeutically effective amount of a compound according to claim 1, or a pharmaceutically acceptable salt thereof, and a therapeutically effective amount of imipenem or a pharmaceutically acceptable salt thereof, cilastatin or a pharmaceutically acceptable salt thereof, and relebactam or a pharmaceutically acceptable salt thereof.

24. The method of claim 19, wherein the bacterial infection is due to Pseudomonas spp., Klebsiella spp., Enterobacter spp., Escherichi spp., Morganella spp., Citrobacter spp., Serratia, spp. or Acintetobacter spp.
